SEQUENCE LISTING (160 sequences)

SEQ ID NO: 1 - DNA, 2019 bp, Bacillus thuringiensis
  CDS (1)..(2019)
atgaatcgaaataatcaaaatgaatatgaaattattgatgccccccattgtgggtgtccatcagatgacgatgtgaggtatcctttggcaagtgacccaaatgcagcgttacaaaatatgaactataaagattacttacaaatgacagatgaggactacactgattcttatataaatcctagtttatctattagtggtagagatgcagttcagactgcgcttactgttgttgggagaatactcggggctttaggtgttccgttttctggacaaatagtgagtttttatcaattccttttaaatacactgtggccagttaatgatacagctatatgggaagctttcatgcgacaggtggaggaacttgtcaatcaacaaataacagaatttgcaagaaatcaggcacttgcaagattgcaaggattaggagactcttttaatgtatatcaacgttcccttcaaaattggttggctgatcgaaatgatacacgaaatttaagtgttgttcgtgctcaatttatagctttagaccttgattttgttaatgctattccattgtttgcagtaaatggacagcaggttccattactgtcagtatatgcacaagctgtgaatttacatttgttattattaaaagatgcatctctttttggagaaggatggggattcacacagggggaaatttccacatattatgaccgtcaattggaactaaccgctaagtacactaattactgtgaaacttggtataatacaggtttagatcgtttaagaggaacaaatactgaaagttggttaagatatcatcaattccgtagagaaatgactttagtggtattagatgttgtggcgctatttccatattatgatgtacgactttatccaacgggatcaaacccacagcttacacgtgaggtatatacagatccgattgtatttaatccaccagctaatgttggactttgccgacgttggggtactaatccctataatactttttctgagctcgaaaatgccttcattcgcccaccacatctttttgataggctgaatagcttaacaatcagcagtaatcgatttccagtttcatctaattttatggattattggtcaggacatacgttacgccgtagttatctgaacgattcagcagtacaagaagatagttatggcctaattacaaccacaagagcaacaattaatcccggagttgatggaacaaaccgcatagagtcaacggcagtagattttcgttctgcattgataggtatatatggcgtgaatagagcttcttttgtcccaggaggcttgtttaatggtacgacttctcctgctaatggaggatgtagagatctctatgatacaaatgatgaattaccaccagatgaaagtaccggaagttcaacccatagactatctcatgttaccttttttagctttcaaactaatcaggctggatctatagctaatgcaggaagtgtacctacttatgtttggacccgtcgtgatgtggaccttaataatacgattaccccaaatagaattacacaattaccattggtaaaggcatctgcacctgtttcgggtactacggtcttaaaaggtccaggatttacaggagggggtatactccgaagaacaactaatggcacatttggaacgttaagagtaacggttaattcaccattaacacaacaatatcgcctaagagttcgttttgcctcaacaggaaatttcagtataagggtactccgtggaggggtttctatcggtgatgttagattagggagcacaatgaacagagggcaggaactaacttacgaatcctttttcacaagagagtttactactactggtccgttcaatccgccttttacatttacacaagctcaagagattctaacagtgaatgcagaaggtgttagcaccggtggtgaatattatatagatagaattgaaattgtccctgtgaatccggcacgagaagcggaagaggatttagaagcggcgaagaaagcg

SEQ ID NO: 2 - protein, 673 aa, Bacillus thuringiensis
MNRNNQNEYEIIDAPHCGCPSDDDVRYPLASDPNAALQNMNYKDYLQMTDEDYTDSYINPSLSISGRDAVQTALTVVGRILGALGVPFSGQIVSFYQFLLNTLWPVNDTAIWEAFMRQVEELVNQQITEFARNQALARLQGLGDSFNVYQRSLQNWLADRNDTRNLSVVRAQFIALDLDFVNAIPLFAVNGQQVPLLSVYAQAVNLHLLLLKDASLFGEGWGFTQGEISTYYDRQLELTAKYTNYCETWYNTGLDRLRGTNTESWLRYHQFRREMTLVVLDVVALFPYYDVRLYPTGSNPQLTREVYTDPIVFNPPANVGLCRRWGTNPYNTFSELENAFIRPPHLFDRLNSLTISSNRFPVSSNFMDYWSGHTLRRSYLNDSAVQEDSYGLITTTRATINPGVDGTNRIESTAVDFRSALIGIYGVNRASFVPGGLFNGTTSPANGGCRDLYDTNDELPPDESTGSSTHRLSHVTFFSFQTNQAGSIANAGSVPTYVWTRRDVDLNNTITPNRITQLPLVKASAPVSGTTVLKGPGFTGGGILRRTTNGTFGTLRVTVNSPLTQQYRLRVRFASTGNFSIRVLRGGVSIGDVRLGSTMNRGQELTYESFFTREFTTTGPFNPPFTFTQAQEILTVNAEGVSTGGEYYIDRIEIVPVNPAREAEEDLEAAKKA

SEQ ID NO: 3 - DNA, 2019 bp, Artificial sequence
  Artificial sequence description Cry9Ca1 Leu- 164
atgaatcgaaataatcaaaatgaatatgaaattattgatgccccccattgtgggtgtccatcagatgacgatgtgaggtatcctttggcaagtgacccaaatgcagcgttacaaaatatgaactataaagattacttacaaatgacagatgaggactacactgattcttatataaatcctagtttatctattagtggtagagatgcagttcagactgcgcttactgttgttgggagaatactcggggctttaggtgttccgttttctggacaaatagtgagtttttatcaattccttttaaatacactgtggccagttaatgatacagctatatgggaagctttcatgcgacaggtggaggaacttgtcaatcaacaaataacagaatttgcaagaaatcaggcacttgcaagattgcaaggattaggagactcttttaatgtatatcaacgttcccttcaaaattggttggctgatcgaaatgatacattaaatttaagtgttgttcgtgctcaatttatagctttagaccttgattttgttaatgctattccattgtttgcagtaaatggacagcaggttccattactgtcagtatatgcacaagctgtgaatttacatttgttattattaaaagatgcatctctttttggagaaggatggggattcacacagggggaaatttccacatattatgaccgtcaattggaactaaccgctaagtacactaattactgtgaaacttggtataatacaggtttagatcgtttaagaggaacaaatactgaaagttggttaagatatcatcaattccgtagagaaatgactttagtggtattagatgttgtggcgctatttccatattatgatgtacgactttatccaacgggatcaaacccacagcttacacgtgaggtatatacagatccgattgtatttaatccaccagctaatgttggactttgccgacgttggggtactaatccctataatactttttctgagctcgaaaatgccttcattcgcccaccacatctttttgataggctgaatagcttaacaatcagcagtaatcgatttccagtttcatctaattttatggattattggtcaggacatacgttacgccgtagttatctgaacgattcagcagtacaagaagatagttatggcctaattacaaccacaagagcaacaattaatcccggagttgatggaacaaaccgcatagagtcaacggcagtagattttcgttctgcattgataggtatatatggcgtgaatagagcttcttttgtcccaggaggcttgtttaatggtacgacttctcctgctaatggaggatgtagagatctctatgatacaaatgatgaattaccaccagatgaaagtaccggaagttcaacccatagactatctcatgttaccttttttagctttcaaactaatcaggctggatctatagctaatgcaggaagtgtacctacttatgtttggacccgtcgtgatgtggaccttaataatacgattaccccaaatagaattacacaattaccattggtaaaggcatctgcacctgtttcgggtactacggtcttaaaaggtccaggatttacaggagggggtatactccgaagaacaactaatggcacatttggaacgttaagagtaacggttaattcaccattaacacaacaatatcgcctaagagttcgttttgcctcaacaggaaatttcagtataagggtactccgtggaggggtttctatcggtgatgttagattagggagcacaatgaacagagggcaggaactaacttacgaatcctttttcacaagagagtttactactactggtccgttcaatccgccttttacatttacacaagctcaagagattctaacagtgaatgcagaaggtgttagcaccggtggtgaatattatatagatagaattgaaattgtccctgtgaatccggcacgagaagcggaagaggatttagaagcggcgaagaaagcg

SEQ ID NO: 4 - protein, 673 aa, Artificial sequence
  Artificial sequence description Cry9Ca1 Leu- 164
MNRNNQNEYEIIDAPHCGCPSDDDVRYPLASDPNAALQNMNYKDYLQMTDEDYTDSYINPSLSISGRDAVQTALTVVGRILGALGVPFSGQIVSFYQFLLNTLWPVNDTAIWEAFMRQVEELVNQQITEFARNQALARLQGLGDSFNVYQRSLQNWLADRNDTLNLSVVRAQFIALDLDFVNAIPLFAVNGQQVPLLSVYAQAVNLHLLLLKDASLFGEGWGFTQGEISTYYDRQLELTAKYTNYCETWYNTGLDRLRGTNTESWLRYHQFRREMTLVVLDVVALFPYYDVRLYPTGSNPQLTREVYTDPIVFNPPANVGLCRRWGTNPYNTFSELENAFIRPPHLFDRLNSLTISSNRFPVSSNFMDYWSGHTLRRSYLNDSAVQEDSYGLITTTRATINPGVDGTNRIESTAVDFRSALIGIYGVNRASFVPGGLFNGTTSPANGGCRDLYDTNDELPPDESTGSSTHRLSHVTFFSFQTNQAGSIANAGSVPTYVWTRRDVDLNNTITPNRITQLPLVKASAPVSGTTVLKGPGFTGGGILRRTTNGTFGTLRVTVNSPLTQQYRLRVRFASTGNFSIRVLRGGVSIGDVRLGSTMNRGQELTYESFFTREFTTTGPFNPPFTFTQAQEILTVNAEGVSTGGEYYIDRIEIVPVNPAREAEEDLEAAKKA

SEQ ID NO: 5 - DNA, 2019 bp, Artificial sequence
  Artificial sequence description Cry9Ca1 Phe- 164
atgaatcgaaataatcaaaatgaatatgaaattattgatgccccccattgtgggtgtccatcagatgacgatgtgaggtatcctttggcaagtgacccaaatgcagcgttacaaaatatgaactataaagattacttacaaatgacagatgaggactacactgattcttatataaatcctagtttatctattagtggtagagatgcagttcagactgcgcttactgttgttgggagaatactcggggctttaggtgttccgttttctggacaaatagtgagtttttatcaattccttttaaatacactgtggccagttaatgatacagctatatgggaagctttcatgcgacaggtggaggaacttgtcaatcaacaaataacagaatttgcaagaaatcaggcacttgcaagattgcaaggattaggagactcttttaatgtatatcaacgttcccttcaaaattggttggctgatcgaaatgatacatttaatttaagtgttgttcgtgctcaatttatagctttagaccttgattttgttaatgctattccattgtttgcagtaaatggacagcaggttccattactgtcagtatatgcacaagctgtgaatttacatttgttattattaaaagatgcatctctttttggagaaggatggggattcacacagggggaaatttccacatattatgaccgtcaattggaactaaccgctaagtacactaattactgtgaaacttggtataatacaggtttagatcgtttaagaggaacaaatactgaaagttggttaagatatcatcaattccgtagagaaatgactttagtggtattagatgttgtggcgctatttccatattatgatgtacgactttatccaacgggatcaaacccacagcttacacgtgaggtatatacagatccgattgtatttaatccaccagctaatgttggactttgccgacgttggggtactaatccctataatactttttctgagctcgaaaatgccttcattcgcccaccacatctttttgataggctgaatagcttaacaatcagcagtaatcgatttccagtttcatctaattttatggattattggtcaggacatacgttacgccgtagttatctgaacgattcagcagtacaagaagatagttatggcctaattacaaccacaagagcaacaattaatcccggagttgatggaacaaaccgcatagagtcaacggcagtagattttcgttctgcattgataggtatatatggcgtgaatagagcttcttttgtcccaggaggcttgtttaatggtacgacttctcctgctaatggaggatgtagagatctctatgatacaaatgatgaattaccaccagatgaaagtaccggaagttcaacccatagactatctcatgttaccttttttagctttcaaactaatcaggctggatctatagctaatgcaggaagtgtacctacttatgtttggacccgtcgtgatgtggaccttaataatacgattaccccaaatagaattacacaattaccattggtaaaggcatctgcacctgtttcgggtactacggtcttaaaaggtccaggatttacaggagggggtatactccgaagaacaactaatggcacatttggaacgttaagagtaacggttaattcaccattaacacaacaatatcgcctaagagttcgttttgcctcaacaggaaatttcagtataagggtactccgtggaggggtttctatcggtgatgttagattagggagcacaatgaacagagggcaggaactaacttacgaatcctttttcacaagagagtttactactactggtccgttcaatccgccttttacatttacacaagctcaagagattctaacagtgaatgcagaaggtgttagcaccggtggtgaatattatatagatagaattgaaattgtccctgtgaatccggcacgagaagcggaagaggatttagaagcggcgaagaaagcg

SEQ ID NO: 6 - protein, 673 aa, Artificial sequence
  Artificial sequence description Cry9Ca1 Phe- 164
MNRNNQNEYEIIDAPHCGCPSDDDVRYPLASDPNAALQNMNYKDYLQMTDEDYTDSYINPSLSISGRDAVQTALTVVGRILGALGVPFSGQIVSFYQFLLNTLWPVNDTAIWEAFMRQVEELVNQQITEFARNQALARLQGLGDSFNVYQRSLQNWLADRNDTFNLSVVRAQFIALDLDFVNAIPLFAVNGQQVPLLSVYAQAVNLHLLLLKDASLFGEGWGFTQGEISTYYDRQLELTAKYTNYCETWYNTGLDRLRGTNTESWLRYHQFRREMTLVVLDVVALFPYYDVRLYPTGSNPQLTREVYTDPIVFNPPANVGLCRRWGTNPYNTFSELENAFIRPPHLFDRLNSLTISSNRFPVSSNFMDYWSGHTLRRSYLNDSAVQEDSYGLITTTRATINPGVDGTNRIESTAVDFRSALIGIYGVNRASFVPGGLFNGTTSPANGGCRDLYDTNDELPPDESTGSSTHRLSHVTFFSFQTNQAGSIANAGSVPTYVWTRRDVDLNNTITPNRITQLPLVKASAPVSGTTVLKGPGFTGGGILRRTTNGTFGTLRVTVNSPLTQQYRLRVRFASTGNFSIRVLRGGVSIGDVRLGSTMNRGQELTYESFFTREFTTTGPFNPPFTFTQAQEILTVNAEGVSTGGEYYIDRIEIVPVNPAREAEEDLEAAKKA

SEQ ID NO: 7 - DNA, 2019 bp, Artificial sequence
  Artificial sequence description Cry9Ca1 Glu- 164
atgaatcgaaataatcaaaatgaatatgaaattattgatgccccccattgtgggtgtccatcagatgacgatgtgaggtatcctttggcaagtgacccaaatgcagcgttacaaaatatgaactataaagattacttacaaatgacagatgaggactacactgattcttatataaatcctagtttatctattagtggtagagatgcagttcagactgcgcttactgttgttgggagaatactcggggctttaggtgttccgttttctggacaaatagtgagtttttatcaattccttttaaatacactgtggccagttaatgatacagctatatgggaagctttcatgcgacaggtggaggaacttgtcaatcaacaaataacagaatttgcaagaaatcaggcacttgcaagattgcaaggattaggagactcttttaatgtatatcaacgttcccttcaaaattggttggctgatcgaaatgatacagaaaatttaagtgttgttcgtgctcaatttatagctttagaccttgattttgttaatgctattccattgtttgcagtaaatggacagcaggttccattactgtcagtatatgcacaagctgtgaatttacatttgttattattaaaagatgcatctctttttggagaaggatggggattcacacagggggaaatttccacatattatgaccgtcaattggaactaaccgctaagtacactaattactgtgaaacttggtataatacaggtttagatcgtttaagaggaacaaatactgaaagttggttaagatatcatcaattccgtagagaaatgactttagtggtattagatgttgtggcgctatttccatattatgatgtacgactttatccaacgggatcaaacccacagcttacacgtgaggtatatacagatccgattgtatttaatccaccagctaatgttggactttgccgacgttggggtactaatccctataatactttttctgagctcgaaaatgccttcattcgcccaccacatctttttgataggctgaatagcttaacaatcagcagtaatcgatttccagtttcatctaattttatggattattggtcaggacatacgttacgccgtagttatctgaacgattcagcagtacaagaagatagttatggcctaattacaaccacaagagcaacaattaatcccggagttgatggaacaaaccgcatagagtcaacggcagtagattttcgttctgcattgataggtatatatggcgtgaatagagcttcttttgtcccaggaggcttgtttaatggtacgacttctcctgctaatggaggatgtagagatctctatgatacaaatgatgaattaccaccagatgaaagtaccggaagttcaacccatagactatctcatgttaccttttttagctttcaaactaatcaggctggatctatagctaatgcaggaagtgtacctacttatgtttggacccgtcgtgatgtggaccttaataatacgattaccccaaatagaattacacaattaccattggtaaaggcatctgcacctgtttcgggtactacggtcttaaaaggtccaggatttacaggagggggtatactccgaagaacaactaatggcacatttggaacgttaagagtaacggttaattcaccattaacacaacaatatcgcctaagagttcgttttgcctcaacaggaaatttcagtataagggtactccgtggaggggtttctatcggtgatgttagattagggagcacaatgaacagagggcaggaactaacttacgaatcctttttcacaagagagtttactactactggtccgttcaatccgccttttacatttacacaagctcaagagattctaacagtgaatgcagaaggtgttagcaccggtggtgaatattatatagatagaattgaaattgtccctgtgaatccggcacgagaagcggaagaggatttagaagcggcgaagaaagcg

SEQ ID NO: 8 - protein, 673 aa, Artificial sequence
  Artificial sequence description Cry9Ca1 Glu- 164
MNRNNQNEYEIIDAPHCGCPSDDDVRYPLASDPNAALQNMNYKDYLQMTDEDYTDSYINPSLSISGRDAVQTALTVVGRILGALGVPFSGQIVSFYQFLLNTLWPVNDTAIWEAFMRQVEELVNQQITEFARNQALARLQGLGDSFNVYQRSLQNWLADRNDTENLSVVRAQFIALDLDFVNAIPLFAVNGQQVPLLSVYAQAVNLHLLLLKDASLFGEGWGFTQGEISTYYDRQLELTAKYTNYCETWYNTGLDRLRGTNTESWLRYHQFRREMTLVVLDVVALFPYYDVRLYPTGSNPQLTREVYTDPIVFNPPANVGLCRRWGTNPYNTFSELENAFIRPPHLFDRLNSLTISSNRFPVSSNFMDYWSGHTLRRSYLNDSAVQEDSYGLITTTRATINPGVDGTNRIESTAVDFRSALIGIYGVNRASFVPGGLFNGTTSPANGGCRDLYDTNDELPPDESTGSSTHRLSHVTFFSFQTNQAGSIANAGSVPTYVWTRRDVDLNNTITPNRITQLPLVKASAPVSGTTVLKGPGFTGGGILRRTTNGTFGTLRVTVNSPLTQQYRLRVRFASTGNFSIRVLRGGVSIGDVRLGSTMNRGQELTYESFFTREFTTTGPFNPPFTFTQAQEILTVNAEGVSTGGEYYIDRIEIVPVNPAREAEEDLEAAKKA

SEQ ID NO: 9 - DNA, 2019 bp, Artificial sequence
  Artificial sequence description Cry9Ca1-100%
atgaatcgaaataatcaaaatgaatatgaaattattgaagccccccattgtgggtgtccatcagaagaagaattaaggtatcctttggcaagtgaaccaaatgcagcgttacaaaatatgaactataaagaatacttacaaatgacagaagaggaatacactgaatcttatataaatcctagtttatctattagtggtagagaagcattacagactgcgcttactgttattaggagaatactcggggctttaggtttaccgttttctggacaaatattaagtttttatcaattccttttaaatacactgtttccattaaatgaaacagctatatttgaagctttcatgcgacagttagaggaacttttaaatcaacaaataacagaatttgcaagaaatcaggcacttgcaagattgcaaggattaggagaatcttttaatttatatcaacgttcccttcaaaattttttggctgaacgaaatgaaacacgaaatttaagtttattacgtgctcaatttatagctttagaacttgaatttttaaatgctattccattgtttgcattaaatggacagcagttaccattactgtcattatatgcacaagctttaaatttacatttgttattattaaaagaagcatctctttttggagaaggatttggattcacacagggggaaatttccacatattatgaacgtcaattggaactaaccgctaagtacactaattactgtgaaactttttataatacaggtttagaacgtttaagaggaacaaatactgaaagttttttaagatatcatcaattccgtagagaaatgactttattattattagaattattagcgctatttccatattatgaattacgactttatccaacgggatcaaacccacagcttacacgtgagttatatacagaaccgattttatttaatccaccagctaatttaggactttgccgacgttttggtactaatccctataatactttttctgagctcgaaaatgccttcattcgcccaccacatctttttgaaaggctgaatagcttaacaatcagcagtaatcgatttccattatcatctaattttatggaatatttttcaggacatacgttacgccgtagttatctgaacgaatcagcattacaagaagaaagttatggcctaattacaaccacaagagcaacaattaatcccggattagaaggaacaaaccgcatagagtcaacggcattagaatttcgttctgcattgataggtatatatggcttaaatagagcttcttttttaccaggaggcttgtttaatggtacgacttctcctgctaatggaggatgtagagaactctatgaaacaaatgaagaattaccaccagaagaaagtaccggaagttcaacccatagactatctcatttaaccttttttagctttcaaactaatcaggctggatctatagctaatgcaggaagtttacctacttatttatttacccgtcgtgaattagaacttaataatacgattaccccaaatagaattacacaattaccattgttaaaggcatctgcacctttatcgggtactacgttattaaaaggtccaggatttacaggagggggtatactccgaagaacaactaatggcacatttggaacgttaagattaacgttaaattcaccattaacacaacaatatcgcctaagattacgttttgcctcaacaggaaatttcagtataaggttactccgtggagggttatctatcggtgaattaagattagggagcacaatgaacagagggcaggaactaacttacgaatcctttttcacaagagagtttactactactggtccgttcaatccgccttttacatttacacaagctcaagagattctaacattaaatgcagaaggtttaagcaccggtggtgaatattatatagaaagaattgaaattttacctttaaatccggcacgagaagcggaagaggaattagaagcggcgaagaaagcg

SEQ ID NO: 10 - protein, 673 aa, Artificial sequence
  Artificial sequence description Cry9Ca1-100%
MNRNNQNEYEIIEAPHCGCPSEEELRYPLASEPNAALQNMNYKEYLQMTEEEYTESYINPSLSISGREALQTALTVIRRILGALGLPFSGQILSFYQFLLNTLFPLNETAIFEAFMRQLEELLNQQITEFARNQALARLQGLGESFNLYQRSLQNFLAERNETRNLSLLRAQFIALELEFLNAIPLFALNGQQLPLLSLYAQALNLHLLLLKEASLFGEGFGFTQGEISTYYERQLELTAKYTNYCETFYNTGLERLRGTNTESFLRYHQFRREMTLLLLELLALFPYYELRLYPTGSNPQLTRELYTEPILFNPPANLGLCRRFGTNPYNTFSELENAFIRPPHLFERLNSLTISSNRFPLSSNFMEYFSGHTLRRSYLNESALQEESYGLITTTRATINPGLEGTNRIESTALEFRSALIGIYGLNRASFLPGGLFNGTTSPANGGCRELYETNEELPPEESTGSSTHRLSHLTFFSFQTNQAGSIANAGSLPTYLFTRRELELNNTITPNRITQLPLLKASAPLSGTTLLKGPGFTGGGILRRTTNGTFGTLRLTLNSPLTQQYRLRLRFASTGNFSIRLLRGGLSIGELRLGSTMNRGQELTYESFFTREFTTTGPFNPPFTFTQAQEILTLNAEGLSTGGEYYIERIEILPLNPAREAEEELEAAKKA

SEQ ID NO: 11 - DNA, 2019 bp, Artificial sequence
  Artificial sequence description Cry9Ca1-25%
atgaatcgaaataatcaaaatgaatatgaaattattgatgccccccattgtgggtgtccatcagatgacgatgtgaggtatcctttggcaagtgacccaaatgcagcgttacaaaatatgaactataaagattacttacaaatgacagatgaggactacactgattcttatataaatcctagtttatctattagtggtagagaagcattacagactgcgcttacgttattagggagaatactcggggctttaggtgttccgttttctggacaaatattaagtttttatcaattccttttaaatacactgtggccagttaatgatacagctatatgggaagctttcatgcgacaggtggaggaacttgtcaatcaacaaataacagaatttgcaagaaatcaggcacttgcaagattgcaaggattaggagaatcttttaatgtatatcaacgttcccttcaaaattggttggctgatcgaaatgatacacgaaatttaagtttattacgtgctcaatttatagctttagaccttgattttgttaatgctattccattgtttgcagtaaatggacagcaggttccattactgtcagtatatgcacaagctttaaatttacatttgttattattaaaagaagcatctctttttggagaaggatggggattcacacagggggaaatttccacatattatgaacgtcaattggaactaaccgctaagtacactaattactgtgaaacttggtataatacaggtttagaacgtttaagaggaacaaatactgaaagttttttaagatatcatcaattccgtagagaaatgactttagtggtattagatgttgtggcgctatttccatattatgatgtacgactttatccaacgggatcaaacccacagcttacacgtgaggtatatacagatccgattgtatttaatccaccagctaatttaggactttgccgacgttggggtactaatccctataatactttttctgagctcgaaaatgccttcattcgcccaccacatctttttgaaaggctgaatagcttaacaatcagcagtaatcgatttccagtttcatctaattttatggaatatttttcaggacatacgttacgccgtagttatctgaacgattcagcagtacaagaagatagttatggcctaattacaaccacaagagcaacaattaatcccggagttgatggaacaaaccgcatagagtcaacggcagtagattttcgttctgcattgataggtatatatggcgtgaatagagcttcttttgtcccaggaggcttgtttaatggtacgacttctcctgctaatggaggatgtagagatctctatgatacaaatgatgaattaccaccagatgaaagtaccggaagttcaacccatagactatctcatttaaccttttttagctttcaaactaatcaggctggatctatagctaatgcaggaagtgtacctacttatgtttggacccgtcgtgatgtggaccttaataatacgattaccccaaatagaattacacaattaccattggtaaaggcatctgcacctgtttcgggtactacggtcttaaaaggtccaggatttacaggagggggtatactccgaagaacaactaatggcacatttggaacgttaagagtaacggttaattcaccattaacacaacaatatcgcctaagattacgttttgcctcaacaggaaatttcagtataagggtactccgtggaggggtttctatcggtgatgttagattagggagcacaatgaacagagggcaggaactaacttacgaatcctttttcacaagagagtttactactactggtccgttcaatccgccttttacatttacacaagctcaagagattctaacagtgaatgcagaaggtgttagcaccggtggtgaatattatatagatagaattgaaattgtccctgtgaatccggcacgagaagcggaagaggatttagaagcggcgaagaaagcg

SEQ ID NO: 12 - protein, 673 aa, Artificial sequence
  Artificial sequence description Cry9Ca1-25%
MNRNNQNEYEIIDAPHCGCPSDDDVRYPLASDPNAALQNMNYKDYLQMTDEDYTDSYINPSLSISGREALQTALTLLGRILGALGVPFSGQILSFYQFLLNTLWPVNDTAIWEAFMRQVEELVNQQITEFARNQALARLQGLGESFNVYQRSLQNWLADRNDTRNLSLLRAQFIALDLDFVNAIPLFAVNGQQVPLLSVYAQALNLHLLLLKEASLFGEGWGFTQGEISTYYERQLELTAKYTNYCETWYNTGLERLRGTNTESFLRYHQFRREMTLVVLDVVALFPYYDVRLYPTGSNPQLTREVYTDPIVFNPPANLGLCRRWGTNPYNTFSELENAFIRPPHLFERLNSLTISSNRFPVSSNFMEYFSGHTLRRSYLNDSAVQEDSYGLITTTRATINPGVDGTNRIESTAVDFRSALIGIYGVNRASFVPGGLFNGTTSPANGGCRDLYDTNDELPPDESTGSSTHRLSHLTFFSFQTNQAGSIANAGSVPTYVWTRRDVDLNNTITPNRITQLPLVKASAPVSGTTVLKGPGFTGGGILRRTTNGTFGTLRVTVNSPLTQQYRLRLRFASTGNFSIRVLRGGVSIGDVRLGSTMNRGQELTYESFFTREFTTTGPFNPPFTFTQAQEILTVNAEGVSTGGEYYIDRIEIVPVNPAREAEEDLEAAKKA

SEQ ID NO: 13 - DNA, 30 bp, Artificial sequence
  Artificial sequence description mutant 1
gaattaaatgaatttttaaatttaagtgtt

SEQ ID NO: 14 - DNA, 30 bp, Artificial sequence
  Artificial sequence description mutant 2
gaattaaatgaattattaaatttaagtgtt

SEQ ID NO: 15 - DNA, 30 bp, Artificial sequence
  Artificial sequence description mutant 3
gaattattagaatttttattattaagtgtt

SEQ ID NO: 16 - DNA, 30 bp, Artificial sequence
  Artificial sequence description mutant 4
gaattattagaattattattattaagtgtt

SEQ ID NO: 17 - DNA, 30 bp, Artificial sequence
  Artificial sequence description mutant 5
gaattattagaagaattattattaagtgtt

SEQ ID NO: 18 - DNA, 30 bp, Artificial sequence
  Artificial sequence description mutant 6
gaacgattagaatttttattattaagtgtt

SEQ ID NO: 19 - DNA, 30 bp, Artificial sequence
  Artificial sequence description mutant 7
gaacgattagaattattattattaagtgtt

SEQ ID NO: 20 - DNA, 30 bp, Artificial sequence
  Artificial sequence description mutant 8
gaattagaagaattattattattaagtgtt

SEQ ID NO: 21 - DNA, 30 bp, Artificial sequence
  Artificial sequence description mutant 9
gaattattagaagaagaagaattaagtgtt

SEQ ID NO: 22 - DNA, 33 bp, Artificial sequence
  Artificial sequence description mutant 10
tttttattaaatttattttttttaccattactg

SEQ ID NO: 23 - DNA, 33 bp, Artificial sequence
  Artificial sequence description mutant11
tttttattaaatttagaagaattaccattactg

SEQ ID NO: 24 - DNA, 33 bp, Artificial sequence
  Artificial sequence description mutant 12
tttgaagaaaatttagaagaattaccattactg

SEQ ID NO: 25 - DNA, 33 bp, Artificial sequence
  Artificial sequence description mutant 13
tttgaagaaaattttttattatttccattactg

SEQ ID NO: 26 - DNA, 33 bp, Artificial sequence
  Artificial sequence description mutant 14
tttgaagaaaattttgaagaatttccattactg

SEQ ID NO: 27 - DNA, 33 bp, Artificial sequence
  Artificial sequence description mutant 15
tttttattaaattttgaagaatttccattactg

SEQ ID NO: 28 - DNA, 33 bp, Artificial sequence
  Artificial sequence description mutant 16
tttttattaaatgaattttttgaaccattactg

SEQ ID NO: 29 - DNA, 24 bp, Artificial sequence
  Artificial sequence description mutant 17
ctttttttagaattatttttattc

SEQ ID NO: 30 - DNA, 24 bp, Artificial sequence
  Artificial sequence description mutant 18
ctttttttattattatttttattc

SEQ ID NO: 31 - DNA, 24 bp, Artificial sequence
  Artificial sequence description mutant 19
ctttttttagaagaatttgaatta

SEQ ID NO: 32 - DNA, 24 bp, Artificial sequence
  Artificial sequence description mutant 20
ctttttgaagaagaatttgaatta

SEQ ID NO: 33 - DNA, 24 bp, Artificial sequence
  Artificial sequence description mutant 21
ctttttgaagaattatttgaagaa

SEQ ID NO: 34 - DNA, 15 bp, Artificial sequence
  Artificial sequence description mutant 22
ttattagaattaaat

SEQ ID NO: 35 - DNA, 15 bp, Artificial sequence
  Artificial sequence description mutant 23
ttattatttttaaat

SEQ ID NO: 36 - DNA, 15 bp, Artificial sequence
  Artificial sequence description mutant 24
ttagaattattaaat

SEQ ID NO: 37 - DNA, 15 bp, Artificial sequence
  Artificial sequence description mutant 25
ttattattttttaat

SEQ ID NO: 38 - DNA, 15 bp, Artificial sequence
  Artificial sequence description mutant 26
ttagaagaattaaat

SEQ ID NO: 39 - DNA, 15 bp, Artificial sequence
  Artificial sequence description mutant 27
ttagaatttttaaat

SEQ ID NO: 40 - DNA, 15 bp, Artificial sequence
  Artificial sequence description mutant 28
ttagaatttgaaaat

SEQ ID NO: 41 - DNA, 15 bp, Artificial sequence
  Artificial sequence description mutant 29
ttagaagaagaaaat

SEQ ID NO: 42 - DNA, 33 bp, Artificial sequence
  Artificial sequence description oligonucleotide 1
gatcgaaatgatacattaaatttaagtgttgtt

SEQ ID NO: 43 - DNA, 33 bp, Artificial sequence
  Artificial sequence description oligonucleotide 2
gatcgaaatgatacatttaatttaagtgttgtt

SEQ ID NO: 44 - DNA, 33 bp, Artificial sequence
  Artificial sequence description oligonucleotide 3
gatcgaaatgatacagaaaatttaagtgttgtt

SEQ ID NO: 45 - DNA, 33 bp, Artificial sequence
  Artificial sequence description oligonucleotide 4
cgaaatgatacacgattattaagtgttgttcgt

SEQ ID NO: 46 - DNA, 33 bp, Artificial sequence
  Artificial sequence description oligonucleotide 5
cgaaatgatacacgagaattaagtgttgttcgt

SEQ ID NO: 47 - DNA, 39 bp, Artificial sequence
  Artificial sequence description oligonucleotide 6
ttggctgatcgaaatgaatttttaaatttaagtgttgtt

SEQ ID NO: 48 - DNA, 39 bp, Artificial sequence
  Artificial sequence description oligonucleotide 7
ttggctgatcgaaatgaatttttattattaagtgttgtt

SEQ ID NO: 49 - DNA, 39 bp, Artificial sequence
  Artificial sequence description oligonucleotide 8
ttggctgatcgaaatgaattattaaatttaagtgttgtt

SEQ ID NO: 50 - DNA, 39 bp, Artificial sequence
  Artificial sequence description oligonucleotide 9
ttggctgatcgaaatgaattattattattaagtgttgtt

SEQ ID NO: 51 - DNA, 39 bp, Artificial sequence
  Artificial sequence description oligonucleotide 10
ttggctgatcgaaatgaagaagaagaattaagtgttgtt

SEQ ID NO: 52 - DNA, 39 bp, Artificial sequence
  Artificial sequence description oligonucleotide 11
ttggctgatcgaaatgaagaattattattaagtgttgtt

SEQ ID NO: 53 - DNA, 36 bp, Artificial sequence
  Artificial sequence description oligonucleotide 12
caaaattggttggctgaattaaatgaattattaaat

SEQ ID NO: 54 - DNA, 36 bp, Artificial sequence
  Artificial sequence description oligonucleotide 13
caaaattggttggctgaattaaatgaatttttaaat

SEQ ID NO: 55 - DNA, 39 bp, Artificial sequence
  Artificial sequence description oligonucleotide 14
caaaattggttggctgaattattagaatttttattatta

SEQ ID NO: 56 - DNA, 39 bp, Artificial sequence
  Artificial sequence description oligonucleotide 15
caaaattggttggctgaattattagaattattattatta

SEQ ID NO: 57 - DNA, 39 bp, Artificial sequence
  Artificial sequence description oligonucleotide 16
caaaattggttggctgaattattagaagaattattatta

SEQ ID NO: 58 - DNA, 39 bp, Artificial sequence
  Artificial sequence description oligonucleotide 17
caaaattggttggctgaacgattagaatttttattatta

SEQ ID NO: 59 - DNA, 39 bp, Artificial sequence
  Artificial sequence description oligonucleotide 18
caaaattggttggctgaacgattagaattattattatta

SEQ ID NO: 60 - DNA, 39 bp, Artificial sequence
  Artificial sequence description oligonucleotide 19
caaaattggttggctgaattagaagaattattattatta

SEQ ID NO: 61 - DNA, 39 bp, Artificial sequence
  Artificial sequence description oligonucleotide 20
caaaattggttggctgaattattagaagaagaagaatta

SEQ ID NO: 62 - DNA, 36 bp, Artificial sequence
  Artificial sequence description oligonucleotide 21
gctattccattgtttttattaaatggacagcaggtt

SEQ ID NO: 63 - DNA, 36 bp, Artificial sequence
  Artificial sequence description oligonucleotide 22
gctattccattgtttgaagaaaatggacagcaggtt

SEQ ID NO: 64 - DNA, 36 bp, Artificial sequence
  Artificial sequence description oligonucleotide 23
ttattaaatggacagcagttaccattactgtcagta

SEQ ID NO: 65 - DNA, 36 bp, Artificial sequence
  Artificial sequence description oligonucleotide 24
ttattaaatggacagcagtttccattactgtcagta

SEQ ID NO: 66 - DNA, 36 bp, Artificial sequence
  Artificial sequence description oligonucleotide 25
ttattaaatggacagcaggaaccattactgtcagta

SEQ ID NO: 67 - DNA, 36 bp, Artificial sequence
  Artificial sequence description oligonucleotide 26
gaagaaaatggacagcagttaccattactgtcagta

SEQ ID NO: 68 - DNA, 36 bp, Artificial sequence
  Artificial sequence description oligonucleotide 27
gaagaaaatggacagcagtttccattactgtcagta

SEQ ID NO: 69 - DNA, 45 bp, Artificial sequence
  Artificial sequence description oligonucleotide 28
ccattgtttttattaaatttattttttttaccattactgtcagta

SEQ ID NO: 70 - DNA, 45 bp, Artificial sequence
  Artificial sequence description oligonucleotide 29
ccattgtttttattaaatttagaagaattaccattactgtcagta

SEQ ID NO: 71 - DNA, 45 bp, Artificial sequence
  Artificial sequence description oligonucleotide 30
ccattgtttgaagaaaatttagaagaattaccattactgtcagta

SEQ ID NO: 72 - DNA, 45 bp, Artificial sequence
  Artificial sequence description oligonucleotide 31
ccattgtttgaagaaaattttttattatttccattactgtcagta

SEQ ID NO: 73 - DNA, 45 bp, Artificial sequence
  Artificial sequence description oligonucleotide 32
ccattgtttgaagaaaattttgaagaatttccattactgtcagta

SEQ ID NO: 74 - DNA, 45 bp, Artificial sequence
  Artificial sequence description oligonucleotide 33
ccattgtttttattaaattttgaagaatttccattactgtcagta

SEQ ID NO: 75 - DNA, 45 bp, Artificial sequence
  Artificial sequence description oligonucleotide 34
ccattgtttttattaaatgaattttttgaaccattactgtcagta

SEQ ID NO: 76 - DNA, 33 bp, Artificial sequence
  Artificial sequence description oligonucleotide 35
gatgcatctctttttttagaaggatggggattc

SEQ ID NO: 77 - DNA, 36 bp, Artificial sequence
  Artificial sequence description oligonucleotide 36
gatgcatctctttttttattaggatggggattcaca

SEQ ID NO: 78 - DNA, 33 bp, Artificial sequence
  Artificial sequence description oligonucleotide 37
gatgcatctctttttgaagaaggatggggattc

SEQ ID NO: 79 - DNA, 33 bp, Artificial sequence
  Artificial sequence description oligonucleotide 38
ttagaaggatggggattaacacagggggaaatt

SEQ ID NO: 80 - DNA, 33 bp, Artificial sequence
  Artificial sequence description oligonucleotide 39
gaagaaggatggggagaaacacagggggaaatt

SEQ ID NO: 81 - DNA, 45 bp, Artificial sequence
  Artificial sequence description oligonucleotide 40
gcatctctttttttagaattatttttattcacacagggggaaatt

SEQ ID NO: 82 - DNA, 45 bp, Artificial sequence
  Artificial sequence description oligonucleotide 41
gcatctctttttttattattatttttattcacacagggggaaatt

SEQ ID NO: 83 - DNA, 45 bp, Artificial sequence; identical to SEQ ID NO: 81
  Artificial sequence description oligonucleotide 42

SEQ ID NO: 84 - DNA, 45 bp, Artificial sequence
  Artificial sequence description oligonucleotide 43
gcatctctttttgaagaattatttttattcacacagggggaaatt

SEQ ID NO: 85 - DNA, 45 bp, Artificial sequence
  Artificial sequence description oligonucleotide 44
gcatctctttttgaagaattatttttagaaacacagggggaaatt

SEQ ID NO: 86 - DNA, 39 bp, Artificial sequence
  Artificial sequence description oligonucleotide 45
ggtttagatcgtttattagaattaaatactgaaagttgg

SEQ ID NO: 87 - DNA, 39 bp, Artificial sequence
  Artificial sequence description oligonucleotide 46
ggtttagatcgtttattatttttaaatactgaaagttgg

SEQ ID NO: 88 - DNA, 39 bp, Artificial sequence
  Artificial sequence description oligonucleotide 47
ggtttagatcgtttagaattattaaatactgaaagttgg

SEQ ID NO: 89 - DNA, 39 bp, Artificial sequence
  Artificial sequence description oligonucleotide 48
ggtttagatcgtttattattttttaatactgaaagttgg

SEQ ID NO: 90 - DNA, 39 bp, Artificial sequence
  Artificial sequence description oligonucleotide 49
ggtttagatcgtttagaagaattaaatactgaaagttgg

SEQ ID NO: 91 - DNA, 39 bp, Artificial sequence
  Artificial sequence description oligonucleotide 50
ggtttagatcgtttagaatttttaaatactgaaagttgg

SEQ ID NO: 92 - DNA, 39 bp, Artificial sequence
  Artificial sequence description oligonucleotide 51
ggtttagatcgtttagaatttgaaaatactgaaagttgg

SEQ ID NO: 93 - DNA, 39 bp, Artificial sequence
  Artificial sequence description oligonucleotide 52
ggtttagatcgtttagaagaagaaaatactgaaagttgg

SEQ ID NO: 94 - DNA, 30 bp, Artificial sequence
  Artificial sequence description oligonucleotide 53
tgaatatgaaattattgaagccccccattg

SEQ ID NO: 95 - DNA, 40 bp, Artificial sequence
  Artificial sequence description oligonucleotide 54
tgggtgtccatcagaagaagaattaaggtatcctttggca

SEQ ID NO: 96 - DNA, 27 bp, Artificial sequence
  Artificial sequence description oligonucleotide 55
tcctttggcaagtgaaccaaatgcagc

SEQ ID NO: 97 - DNA, 25 bp, Artificial sequence
  Artificial sequence description oligonucleotide 56
gaactataaagaatacttacaaatg

SEQ ID NO: 98 - DNA, 26 bp, Artificial sequence
  Artificial sequence description oligonucleotide 57
caaatgacagaagaggaatacactga

SEQ ID NO: 99 - DNA, 20 bp, Artificial sequence
  Artificial sequence description oligonucleotide 58
tacactgaatcttatataaa

SEQ ID NO: 100 - DNA, 36 bp, Artificial sequence
  Artificial sequence description oligonucleotide 59
tattagtggtagagaagcattacagactgcgcttac

SEQ ID NO: 101 - DNA, 37 bp, Artificial sequence
  Artificial sequence description oligonucleotide 60
cagactgcgcttactgttattagggagaatactcggg

SEQ ID NO: 102 - DNA, 25 bp, Artificial sequence
  Artificial sequence description oligonucleotide 61
gggctttaggtttaccgttttctgg

SEQ ID NO: 103 - DNA, 28 bp, Artificial sequence
  Artificial sequence description oligonucleotide 62
ttctggacaaatattaagtttttatcaa

SEQ ID NO: 104 - DNA, 40 bp, Artificial sequence
  Artificial sequence description oligonucleotide 63
cttttaaatacactgtttccattaaatgaaacagctatat

SEQ ID NO: 105 - DNA, 24 bp, Artificial sequence
  Artificial sequence description oligonucleotide 64
acagctatatttgaagctttcatg

SEQ ID NO: 106 - DNA, 26 bp, Artificial sequence
  Artificial sequence description oligonucleotide 65
ctttcatgcgacagttagaggaactt

SEQ ID NO: 107 - DNA, 26 bp, Artificial sequence
  Artificial sequence description oligonucleotide 66
gaggaacttttaaatcaacaaataac

SEQ ID NO: 108 - DNA, 21 bp, Artificial sequence
  Artificial sequence description oligonucleotide 67
ggattaggagaatcttttaat

SEQ ID NO: 109 - DNA, 23 bp, Artificial sequence
  Artificial sequence description oligonucleotide 68
tcttttaatttatatcaacgttc

SEQ ID NO: 110 - DNA, 21 bp, Artificial sequence
  Artificial sequence description oligonucleotide 69
ccttcaaaattttttggctga

SEQ ID NO: 111 - DNA, 17 bp, Artificial sequence
  Artificial sequence description oligonucleotide 70
ttggctgaacgaaatga

SEQ ID NO: 112 - DNA, 23 bp, Artificial sequence
  Artificial sequence description oligonucleotide 71
cgaaatgaaacacgaaatttaag

SEQ ID NO: 113 - DNA, 37 bp, Artificial sequence
  Artificial sequence description oligonucleotide 72
acacgaaatttaagtttattacgtgctcaatttatag

SEQ ID NO: 114 - DNA, 48 bp, Artificial sequence
  Artificial sequence description oligonucleotide 73
gctcaatttatagctttagaacttgaatttttaaatgctattccattg

SEQ ID NO: 115 - DNA, 27 bp, Artificial sequence
  Artificial sequence description oligonucleotide 74
ccattgtttgcattaaatggacagcag

SEQ ID NO: 116 - DNA, 27 bp, Artificial sequence
  Artificial sequence description oligonucleotide 75
ccattgtttgcattaaatggacagcag

SEQ ID NO: 117 - DNA, 27 bp, Artificial sequence
  Artificial sequence description oligonucleotide 76
ccattactgtcattatatgcacaagct

SEQ ID NO: 118 - DNA, 27 bp, Artificial sequence
  Artificial sequence description oligonucleotide 77
tatgcacaagctttaaatttacatttg

SEQ ID NO: 119 - DNA, 23 bp, Artificial sequence
  Artificial sequence description oligonucleotide 78
ttattaaaagaagcatctctttt

SEQ ID NO: 120 - DNA, 25 bp, Artificial sequence
  Artificial sequence description oligonucleotide 79
tggagaaggatttggattcacacag

SEQ ID NO: 121 - DNA, 24 bp, Artificial sequence
  Artificial sequence description oligonucleotide 80
cacatattatgaacgtcaattgga

SEQ ID NO: 122 - DNA, 28 bp, Artificial sequence
  Artificial sequence description oligonucleotide 81
tactgtgaaactttttataatacaggtt

SEQ ID NO: 123 - DNA, 25 bp, Artificial sequence
  Artificial sequence description oligonucleotide 82
tacaggtttagaacgtttaagagga

SEQ ID NO: 124 - DNA, 28 bp, Artificial sequence
  Artificial sequence description oligonucleotide 83
aatactgaaagttttttaagatatcatc

SEQ ID NO: 125 - DNA, 51 bp, Artificial sequence
  Artificial sequence description oligonucleotide 84
gtagagaaatgactttattattattagaattattagcgctatttccatatt

SEQ ID NO: 126 - DNA, 27 bp, Artificial sequence
  Artificial sequence description oligonucleotide 85
atattatgaattacgactttatccaac

SEQ ID NO: 127 - DNA, 23 bp, Artificial sequence
  Artificial sequence description oligonucleotide 86
cttacacgtgagttatatacaga

SEQ ID NO: 128 - DNA, 29 bp, Artificial sequence
  Artificial sequence description oligonucleotide 87
tatacagaaccgattttatttaatccacc

SEQ ID NO: 129 - DNA, 28 bp, Artificial sequence
  Artificial sequence description oligonucleotide 88
ccaccagctaatttaggactttgccgac

SEQ ID NO: 130 - DNA, 27 bp, Artificial sequence
  Artificial sequence description oligonucleotide 89
ctttgccgacgttttggtactaatccc

SEQ ID NO: 131 - DNA, 23 bp, Artificial sequence
  Artificial sequence description oligonucleotide 90
catctttttgaaaggctgaatag

SEQ ID NO: 132 - DNA, 30 bp, Artificial sequence
  Artificial sequence description oligonucleotide 91
taatcgatttccattatcatctaattttat

SEQ ID NO: 133 - DNA, 36 bp, Artificial sequence
  Artificial sequence description oligonucleotide 92
ctaattttatggaatatttttcaggacatacgttac

SEQ ID NO: 134 - DNA, 33 bp, Artificial sequence
  Artificial sequence description oligonucleotide 93
tagttatctgaacgaatcagcattacaagaaga

SEQ ID NO: 135 - DNA, 20 bp, Artificial sequence
  Artificial sequence description oligonucleotide 94
caagaagaaagttatggcct

SEQ ID NO: 136 - DNA, 35 bp, Artificial sequence
  Artificial sequence description oligonucleotide 95
caattaatcccggattagaaggaacaaaccgcata

SEQ ID NO: 137 - DNA, 30 bp, Artificial sequence
  Artificial sequence description oligonucleotide 96
gagtcaacggcattagaatttcgttctgca

SEQ ID NO: 138 - DNA, 26 bp, Artificial sequence
  Artificial sequence description oligonucleotide 97
ggtatatatggcttaaatagagcttc

SEQ ID NO: 139 - DNA, 30 bp, Artificial sequence
  Artificial sequence description oligonucleotide 98
tagagcttcttttttaccaggaggcttgtt

SEQ ID NO: 140 - DNA, 31 bp, Artificial sequence
  Artificial sequence description oligonucleotide 99
ctgctaatggaggatgtagagaactctatga

SEQ ID NO: 141 - DNA, 17 bp, Artificial sequence
  Artificial sequence description oligonucleotide 100
ctctatgaaacaaatga

SEQ ID NO: 142 - DNA, 20 bp, Artificial sequence
  Artificial sequence description oligonucleotide 101
acaaatgaagaattaccacc

SEQ ID NO: 143 - DNA, 27 bp, Artificial sequence
  Artificial sequence description oligonucleotide 102
attaccaccagaagaaagtaccggaag

SEQ ID NO: 144 - DNA, 30 bp, Artificial sequence
  Artificial sequence description oligonucleotide 103
agactatctcatttaaccttttttagcttt

SEQ ID NO: 145 - DNA, 27 bp, Artificial sequence
  Artificial sequence description oligonucleotide 104
gctaatgcaggaagtttacctacttat

SEQ ID NO: 146 - DNA, 26 bp, Artificial sequence
  Artificial sequence description oligonucleotide 105
cctacttatttatttacccgtcgtga

SEQ ID NO: 147 - DNA, 33 bp, Artificial sequence
  Artificial sequence description oligonucleotide 106
acccgtcgtgaattagaacttaataatacgatt

SEQ ID NO: 148 - DNA, 24 bp, Artificial sequence
  Artificial sequence description oligonucleotide 107
attaccattgttaaaggcatctgc

SEQ ID NO: 149 - DNA, 30 bp, Artificial sequence
  Artificial sequence description oligonucleotide 108
aaggcatctgcacctttatcgggtactacg

SEQ ID NO: 150 - DNA, 29 bp, Artificial sequence
  Artificial sequence description oligonucleotide 109
tcgggtactacgttattaaaaggtccagg

SEQ ID NO: 151 - DNA, 40 bp, Artificial sequence
  Artificial sequence description oligonucleotide 110
acatttggaacgttaagattaacgttaaattcaccattaa

SEQ ID NO: 152 - DNA, 37 bp, Artificial sequence
  Artificial sequence description oligonucleotide 111
cacaacaatatcgcctaagattacgttttgcctcaac

SEQ ID NO: 153 - DNA, 31 bp, Artificial sequence
  Artificial sequence description oligonucleotide 112
aaatttcagtataaggttactccgtggaggg

SEQ ID NO: 154 - DNA, 35 bp, Artificial sequence
  Artificial sequence description oligonucleotide 113
ataagggtactccgtggagggttatctatcggtga

SEQ ID NO: 155 - DNA, 29 bp, Artificial sequence
  Artificial sequence description oligonucleotide 114
tctatcggtgaattaagattagggagcac

SEQ ID NO: 156 - DNA, 30 bp, Artificial sequence
  Artificial sequence description oligonucleotide 115
caagagattctaacattaaatgcagaaggt

SEQ ID NO: 157 - DNA, 32 bp, Artificial sequence
  Artificial sequence description oligonucleotide 116
aatgcagaaggtttaagcaccggtggtgaata

SEQ ID NO: 158 - DNA, 32 bp, Artificial sequence
  Artificial sequence description oligonucleotide 117
gtggtgaatattatatagaaagaattgaaatt

SEQ ID NO: 159 - DNA, 37 bp, Artificial sequence
  Artificial sequence description oligonucleotide 118
agaattgaaattttacctttaaatccggcacgagaag

SEQ ID NO: 160 - DNA, 30 bp, Artificial sequence
  Artificial sequence description oligonucleotide 119
cgagaagcggaagaggaattagaagcggcg

(19) United States
(12) Patent Application Publication
(10) Pub. No.: US 2004/0096934 A1
(43) Pub. Date: May 20, 2004

(54) Pepsin-sensitive modified Bacillus thuringiensis insecticidal toxin

(76) Inventors: Georges Freyssinet, St. Cyr au Mont d'Or (FR); Cecile Rang, St. Georges d'Orques (FR); Roger Frutos, St. Georges d'Orques (FR)

Correspondence Address: BAKER & BOTTS, 30 ROCKEFELLER PLAZA, NEW YORK, NY, 10112

(21) Appl. No.: 10/665,460

(22) Filed: Sep. 19, 2003

Related U.S. Application Data
(63) Continuation of application No. PCT/FR02/00772, filed on Mar. 4, 2002.

(30) Foreign Application Priority Data
Mar. 19, 2001 (FR) 01/03691

(51) Int. Cl. C07K 14/325; C12P 21/06

(52) U.S. Cl. 435/68.1; 530/350

(57) ABSTRACT

The invention relates to the degradation of Bacillus thuringiensis Cry proteins in the digestive tracts of mammals and concerns Bacillus thuringiensis Cry proteins having a peptide sequence that has been modified in such a way as to make said proteins sensitive to the specific enzymes in the digestive tracts of mammals, in particular pepsins. According to the invention, the Cry proteins are modified by inserting pepsin cleavage sites in the peptide sequence thereof. The invention also relates to transformed plants expressing said modified Cry proteins.

### FIELD OF THE INVENTION

The present invention relates to the degradation of Bacillus thuringiensis Cry proteins in the mammalian digestive tract. It relates to Bacillus thuringiensis Cry proteins, the peptide sequence of which has been modified so as to make them sensitive to the specific enzymes in the mammalian digestive tract, in particular to pepsins. According to this invention, the Cry proteins are modified by insertion of pepsin-cleavage sites into their peptide sequence. The invention also relates to transformed plants expressing these modified Cry proteins.

Bacteria of the species Bacillus thuringiensis (hereinafter referred to as Bt) are well known for the insecticidal toxins which they produce. These Gram-positive bacteria form a parasporal crystal protein during their stationary phase, which is greatly responsible for their insecticidal activity. The crystal protein of Bt consists of an insecticidal toxin which is protein in nature, referred to as Cry protein, and encoded by a cry gene. By virtue of its insecticidal properties, this Cry protein was used in protecting crops against pest insects, as an alternative solution to synthetic insecticides. Currently, this agronomic use is essentially implemented by two methods, direct spreading of the product as a biopesticide, and genetic transformation of the plants grown, with a gene encoding a Cry protein. Depending on the strains of Bt from which they are derived, the Cry proteins have insecticidal activities with respect to different insect spectra. The main orders of insects against which the Cry toxins are active are Lepidoptera, Coleoptera and Diptera, but some toxins are effective with respect to other insect orders. All the Cry proteins isolated from the various strains of Bt are grouped together in a classification as a function of their sequence homologies, and they are assigned a code in order to distinguish them (Crickmore et al., 1998, Microbiol. Molec. Biol. Review (62(3), 807-813). The advantage of using these toxins in agriculture therefore lies in their specificity of action with respect to one or more given insect orders, but also in their lack of toxicity with respect to mammals, to birds, to amphibians and to reptiles.

This lack of toxicity with respect to mammals has made it impossible to develop the culturing of transgenic plants expressing a Cry protein, and to use the seeds from these plants for human and animal foodstuffs. However, although they are non-toxic with respect to mammals, some of these proteins are relatively undegraded in the mammalian digestive tract, and this lack of degradation leads to a relatively long persistence of the toxin in the digestive tract of said mammals. In addition, a lack of persistence of Cry proteins in the mammalian digestive tract is one of the criteria taken into account by the administrative authorities (for example the United States Environmental Protection Agency—EPA) which grant marketing authorizations in the food sector for seeds containing these proteins or for products derived from these seeds.

### BACKGROUND OF THE INVENTION

The present invention makes it possible to overcome the drawback mentioned above. This invention is based on the principle according to which the stability of certain Cry proteins in the mammalian digestive tract is thought to be due to a lack of sensitivity of these proteins to the specific enzymes in said digestive tract, in particular to the proteases. The solution to this problem therefore lies in the artificial integration of specific sites, specific to the enzymes of the mammalian digestive tract, into the Cry protein. A subject of the present invention is therefore modified Cry proteins sensitive to the specific enzymes in the mammalian digestive tract, in particular the specific proteases in the mammalian stomach, and more particularly the pepsins. Pepsin is a particular enzyme of the protease family, and it is the major protease present in the mammalian stomach (95% of stomach proteases). It is an aspartic protease which acts at an optimum pH of 2. Pepsin is an enzyme of choice as a source of degradation of Cry proteins since it is not present in the digestive tube of insects, in particular of the Lepidoptera, in which the pH of the digestive tube is between 10 and 11 (Terra, W. B. and C. Ferreira, 1994, Insect digestive enzymes: properties, compartmentalization and function. Comp. Biochem. Physiol. 109B: 1-62). This lack of pepsin in insects therefore guarantees that introducing pepsin-specific sites into the Cry proteins does not present a risk of increasing their degradation in the insect digestive tube. The present invention is therefore a solution to the technical problem set out above, namely an increase in the sensitivity of the Cry proteins to enzymes of the mammalian digestive tract, without alteration of the insecticidal properties of said Cry proteins.

However, the Cry protein is a very organized protein, the activated form of which is made up of three domains, and in which the structure-function relationships are very strong within and between the domains. This high level of organization of the Cry proteins does not permit the random insertion of mutations into the protein. Specifically, the insertion of cleavage sites specific to mammalian stomach enzymes must not alter the insecticidal properties of the toxins.

The Cry proteins are naturally produced by the bacterium Bacillus thuringiensis in the form of inactive protoxins. The natural method of action of these proteins involves solubilization of the crystal protein in the insect intestine, proteolytic degradation of the released protoxin, attachment of the activated toxin to the receptors in the insect intestine, and insertion of the toxin into the apical membrane of the intestinal cells so as to create ion channels or pores. The proteolytic degradation of the protoxin in the insect intestine takes place under the joint action of the alkaline pH and of the serine proteases (essentially trypsin) of the digestive juice (Schnepf et al., 1998).

The Cry toxins consist of three structural domains, domain I, domain II and domain III. Domain I occupies approximately the N-terminal half of the activated toxin. Domains II and III each occupy approximately a quarter of the activated toxin. Domain III is located at the C-terminal end of the activated toxin. Each domain of the Cry protein has its own structure and its own function.

Domain I consists of seven α-helices, 6 amphiphilic helices and a hydrophobic helix, connected to one another via inter-helix loops consisting of a few amino acids. This domain is the transmembrane domain, responsible for the formation of the ion channel or pore (Aronson et al., 1995; Chen et al., 1993; Manoj-Kumar and Aronson, 1999; Masson et al., 1999; Rang et al., 1999; Coux et al., 1999). The formation of the transmembrane pore by the α-helices of domain I in fact involves four Cry proteins which form a complete pore with their four respective α4-helices (Masson et al., 1999). A cylindrical pore of four α4-helices therefore forms. The inside of this pore consists of the hydrophilic faces of the amphiphilic helices; since the negatively charged residues are present on the hydrophilic faces, they are in the lumen of the pore, in aqueous medium, and perform their ion transport function. The outside of the pore consists of the hydrophobic faces which anchor the pore in the lipid membrane. The formation of the pore by the α-helices of domain I therefore involves very strong structure-function relationships and conformational changes over time. The introduction of mutations into the α-helices of domain I therefore has a high probability of disturbing the function of this domain and therefore the activity of the toxin.

Domains II and III of the activated toxin consist of β-sheets, which are themselves also in a very compacted form. These two domains are involved in receptor site recognition (specificity) and in toxin stability (Abdul-Rauf and Ellar, 1999; Dean et al., 1996; Hussain et al., 1996; Lee et al., 1999; Rajamohan et al., 1996, 1998; Wu and Dean, 1996). Domain III exchanges induce changes in specificity (de Maagd et al., 1999). This region is much less conserved, and therefore more variable, than domain I. It is involved in the specificity of each toxin. This variability and these interactions specific to each toxin are involved in the nature of the very specific host spectrum of each toxin and are involved in the recognition of different receptor sites. Recognition of the receptor takes place via loops in domain II and in domain III and the conformation of these loops varies subtly from one toxin to the other as a function of the arrangement and of the interactions between domains II and III. Domain I also interferes with the other two domains and influences the general conformation (Rang et al., 1999, 2001). In addition, very little is known about the structure-function relationships within these two domains, and no information is actually available regarding the conformation required for recognition of a receptor site. It is therefore very difficult to predict consequences of introducing modifications into domains II and III on the specificity, the ability to recognize the receptor sites and the toxicity of the Cry proteins. Moreover, it is known that mutations generated in domains II and III very often induce destabilization of the toxin in insects, leading to a loss of toxicity.

Salt bridges also exist between domains I and II of the Cry proteins. These bridges play an important role in the stability of the toxin and in the functioning thereof. Artificial elimination of these bridges in Cry1Aa1 shows that the protoxins and activated toxins are less stable than the parental protein (Vachon et al., 2000). These salt bridges are present between domain II and the 7-helix of domain I. The acknowledged importance of these bridges implies that mutations in domain II and the α7-helix of domain I have a high risk of disturbing the function of the Cry proteins.

### DETAILED DESCRIPTION OF THE INVENTION

The present invention relates to a pepsin-sensitive modified Cry protein, characterized in that it has at least one additional pepsin cleavage site.

The term “Cry protein” is intended to mean the insecticidal protein produced by a strain of bacterium Bacillus thuringiensis (hereinafter referred to as Bt), the various holotypes of which, which exist and which are to come, are referenced by the Bt classification committee (Crickmore, 2001) and accessible on the Internet site at “www.biols.susx.ac.uk/Home/NeilCrickmore/Bt/index.html.” In particular, this Cry protein is encoded by a cry gene, either naturally by the Bt bacterium, or in a recombinant manner in a host organism transformed with a cry gene or with a gene comprising at least the coding sequence of a Cry protein. The Cry proteins according to the invention also comprise Cry proteins the sequence of which has been artificially modified so as to increase their insecticidal activity or their resistance to treatment conditions. This definition also includes Cry protein fragments which conserve the insecticidal activity, such as the truncated Cry proteins comprising only the N-terminal portion of a complete Cry protein, in particular domain I of this protein (WO 94/05771). Also included are the fused Cry proteins, as described in international patent application WO 94/24264. Preferably, the Cry protein according to the invention is selected from the Cry1, Cry3, Cry4, Cry7, Cry8, Cry9, Cry10, Cry16, Cry17, Cry19 and Cry20 proteins. Preferably, it is the Cry9C protein, and preferably the Cry9Ca1 protein (Lambert et al., Appl. Environm. Microbiol. 62, 80-86; WO 94/05771). In particular, the present invention also fits any Cry protein, the toxicity of which has been improved, such as, for example, those described in patent applications WO 97/49814 or WO 99/00407.

According to the present invention, the Cry protein is modified. The term “modified Cry protein” is intended to mean a Cry protein, the peptide sequence of which is different from the sequence of the native Cry protein from which it is derived. This sequence difference is the result of artificial modifications introduced by genetic engineering, in particular the insertion or the substitution of specific amino acid residues into or in said peptide sequence. In particular, the modified Cry protein is produced by modification of the nucleotide sequence encoding it, in particular by the technique of site-directed mutagenesis well known to those skilled in the art (Hutchinson C. A. et al., 1978, J. Biol. Chem. 253: 6551). Preferably, the modification of the Cry protein consists of an amino acid residue substitution.

The modified Cry protein according to the invention is pepsin-sensitive. The pepsin focuses its proteolytic action on specific cleavage sites consisting of the amino acids leucine, phenylalanine and glutamic acid. The proteolysis takes place on the C-terminal side of the residue concerned. According to the invention, the term “pepsin-sensitive” is intended to mean the property, for the modified Cry protein, of undergoing proteolysis by pepsin. Proteolysis of the Cry protein leads to partial or total loss of the insecticidal activity of said protein. The pepsin-sensitivity can therefore be measured by bringing a modified Cry protein according to the invention into contact, preferably in vitro, with a pepsin, and then measuring the loss of insecticidal activity of said modified Cry protein in comparison with a native Cry protein which has not been modified according to the invention. By way of example, the tests described in Examples 7 and 8 can be used to measure the pepsin sensitivity of a Cry protein according to the invention. Alternatively, the Western blotting technique can also be used to measure said pepsin sensitivity. Using this technique, the sensitivity is measured by observing the structural degradation of the modified Cry protein after contact with a pepsin. This observation consists of the disappearance or the decrease in intensity of a band corresponding to the Cry protein on a gel electrophoresis transfer membrane, compared to a native Cry protein which has not been modified according to the invention. The use of these techniques is part of the general knowledge of those skilled in the art.

The modified Cry protein according to the invention is characterized in that it has at least one additional pepsin cleavage site. The term “pepsin cleavage site” is intended to mean a site consisting of at least one amino acid residue recognized as a site of proteolysis by pepsin. The amino acid residues recognized by pepsin are leucine, phenylalanine or glutamic acid. The expression “additional pepsin cleavage site” is intended to mean an additional cleavage site compared to the native Cry protein as produced by the Bt bacterium.

Preferably, the additional pepsin cleavage site is represented by an amino acid residue selected from leucine, phenylalanine or glutamic acid residues. According to a particular embodiment of the invention, the modified Cry protein has several additional pepsin cleavage sites represented by the same amino acid residue. According to another embodiment of the invention, the modified Cry protein has several additional pepsin cleavage sites represented by different amino acid residues.

According to a particular embodiment of the invention, the modified Cry protein according to the invention is characterized in that it has at least one additional pepsin cleavage site in at least one of the inter-α-helix loops of domain I. The expression “inter-α-helix loops of domain I” is intended to mean the peptide chains linking the seven α-helices of domain I of the Cry proteins as described in Grochulski et al. (1995) and Li et al. (1991). According to the invention, the Cry protein should have at least one additional pepsin cleavage site. In addition, said additional cleavage site is in at least one of the inter-α-helix loops of domain I. The term “additional” is therefore understood to be supplementary compared to the number of pepsin cleavage sites naturally present in the inter-α-helix loops of domain I of the native Cry protein as produced by the Bt bacterium. This definition means that the modified Cry protein according to the invention is characterized in that it has a number of pepsin cleavage sites in its inter-α-helix loops of domain I which is greater than the number of these sites in the same native Cry protein as produced by the Bt bacterium, the difference between said numbers being at least equal to 1.

According to a particular embodiment of the invention, the modified Cry protein according to the invention has at least one pepsin cleavage site in the inter-α-helix loop linking the α3 and α4 helices of domain I.

According to a preferred embodiment of the invention, the modified Cry protein is a modified Cry9C protein. Preferably, the modified Cry protein is a modified Cry9Ca1 protein having a pepsin cleavage site positioned on amino acid residue 164. In particular, the arginine residue naturally present at position 164 on the Cry9Ca1 protein is replaced with an amino acid residue chosen from leucine, phenylalanine and glutamic acid residues, on the Cry9Ca1 protein modified according to the invention. Preferably, the Cry9Ca1 protein modified according to the invention is selected from the Cry proteins the sequences of which are represented by the identifiers SEQ ID NO:4, SEQ ID NO:6 or SEQ ID NO:8.

The present invention also relates to a pepsin-sensitive modified Cry protein, characterized in that the additional pepsin cleavage sites which it possesses are introduced by substituting aspartic acid residues with glutamic acid residues, substituting tryptophan residues with phenylalanine residues, and substituting valine or isoleucine residues with leucine residues. Preferably, the degree of substitution which said modified Cry protein has is 25%. The expression “degree of substitution” is intended to mean the percentage of amino acid residues of the native Cry protein which are replaced with amino acid residues corresponding to pepsin cleavage sites in the modified Cry protein of the invention.

A subject of the present invention is also a method for increasing the pepsin sensitivity of the Cry proteins, characterized in that at least one additional pepsin cleavage site is introduced into said Cry proteins. The expression “increasing the pepsin sensitivity of the Cry proteins” is intended to mean an increase in the pepsin sensitivity of the Cry proteins obtained by said method compared to the corresponding native Cry proteins, this increase resulting in proteolytic destruction and a loss of insecticidal activity of the Cry proteins, these effects possibly being partial or total.

The introduction of at least one pepsin cleavage site is carried out artificially by genetic engineering. In particular, it involves an insertion or a substitution of amino acid residues. Preferably, it involves a substitution. Such a substitution can be readily carried out by the site-directed mutagenesis technique well known to those skilled in the art.

Preferably, the Cry protein to which the method according to the invention applies is selected from the Cry1, Cry3, Cry4, Cry7, Cry8, Cry9, Cry10, Cry16, Cry17, Cry19 and Cry20 proteins. Preferably, it is the Cry9C protein, and preferably the Cry9Ca1 protein.

In particular, the additional pepsin cleavage site is represented by an amino acid residue chosen from leucine, phenylalanine and glutamic acid residues.

According to a particular embodiment of the invention, the method according to the invention is characterized in that at least one additional pepsin cleavage site is introduced into at least one of the inter-α-helix loops of domain I of said Cry protein.

According to another particular embodiment of the invention, the method according to the invention is characterized in that at least one additional pepsin cleavage site is introduced into the inter-α-helix loop linking the α3 and α4 helices of domain I.

According to a preferred embodiment of the invention, the present method applies to a Cry9C protein. Preferably, it applies to a Cry9Ca1 protein, and the additional pepsin cleavage site is introduced by substitution of amino acid residue 164. In particular, the arginine residue naturally present at position 164 on the Cry9Ca1 protein is replaced with an amino acid residue chosen from leucine, phenyl-alanine and glutamic acid residues.

The present invention also relates to a method for increasing the pepsin sensitivity of the Cry proteins, characterized in that the additional pepsin cleavage sites are introduced by substituting aspartic acid residues with glutamic acid residues, substituting tryptophan residues with phenylalanine residues, and substituting valine or isoleucine residues with leucine residues.

Preferably, the degree of substitution introduced into said Cry protein is 25%.

The present invention also relates to a polynucleotide encoding a modified Cry protein according to the invention. According to the present invention, the term “polynucleotide” is intended to mean a natural or artificial nucleotide sequence which may be of the DNA or RNA type, preferably of the DNA type, in particular double-stranded.

The present invention also relates to a chimeric gene comprising, functionally linked to one another, at least one promoter which is functional in a host organism, a polynucleotide encoding a modified Cry protein according to the invention, and a terminator element which is functional in this same host organism. The various elements which a chimeric gene can contain are, firstly, regulatory elements for the transcription, the translation and the maturation of proteins, such as a promoter, a sequence encoding a signal peptide or a transit peptide, or a terminator element constituting a polyadenylation signal and, secondly, a polynucleotide encoding a protein. The expression “functionally linked to one another” means that said elements of the chimeric gene are linked to one another in such a way that the functioning of one of these elements is affected by that of another. By way of example, a promoter is functionally linked to a coding sequence when it is capable of affecting the expression of said coding sequence. The construction of the chimeric gene according to the invention and the assembly of its various elements can be carried out using techniques well known to those skilled in the art, in particular those described in Sambrook et al. (1989, Molecular Cloning: A Laboratory Manual, Nolan C. ed., New York: Cold Spring Harbor Laboratory Press). The choice of the regulatory elements constituting the chimeric gene depends essentially on the host species in which they must function, and those skilled in the art are capable of selecting regulatory elements which are functional in a given host organism. The term “functional” is intended to mean capable of functioning in a given host organism.

According to a particular embodiment of the invention, the chimeric gene contains a “constitutive” promoter. A constitutive promoter according to the present invention is a promoter which induces the expression of a coding sequence in all the tissues of a host organism and continuously, i.e. during the entire duration of the life cycle of said host organism. Some of these promoters can be tissue-specific, i.e. can express the coding sequence continuously, but only in a particular tissue of the host organism. Constitutive promoters can originate from any type of organism. Among the constitutive promoters which may be used in the chimeric gene of the present invention, mention may be made, by way of example, of bacterial promoters, such as that of the octopine synthase gene or that of the nopaline synthase gene, viral promoters, such as that of the gene controlling transcription of the 19S or 35S RNAs of the cauliflower mosaic virus (Odell et al., 1985, Nature, 313, 810-812), or the promoters of the cassava vein mosaic virus (as described in patent application WO 97/48819). Among the promoters of plant origin, mention will be made of the promoter of the ribulose-biscarboxylase/oxygenase (RuBisCO) small subunit gene, the promoter of a histone gene as described in application EP 0 507 698, the promoter of the EF1-α gene (WO 90/02172), the promoter of an actin gene (U.S. Pat. No. 5,641,876), or the promoter of a ubiquitin gene (EP 0342926).

According to another particular embodiment of the invention, the chimeric gene contains an inducible promoter. An inducible promoter is a promoter which only functions, i.e. which only induces expression of a coding sequence, when it is itself induced by an inducing agent. This inducing agent is generally a substance which can be synthesized in the host organism subsequent to a stimulus external to said organism, this external stimulus possibly being physical or chemical, biotic or abiotic in nature. Such promoters are known, such as, for example, the promoter of the plant O-methyltransferase class II (COMT II) gene described in patent application WO 00/56897, the Arabidopsis PR-1 promoter (Lebel et al., 1998, Plant J. 16(2): 223-233), the EAS4 promoter of the tobacco sesquiterpene synthase gene (Yin et al., 1997, Plant Physiol. 115(2), 437-451), or the promoter of the gene encoding 3-hydroxy-3-methylglutaryl coenzyme A reductase (Nelson et al., 1994, Plant Mol. Biol. 25(3): 401-412).

Among the terminator elements which can be used in the chimeric gene of the present invention, mention may, for example, be made of the nos terminator element of the gene encoding Agrobacterium tumefaciens nopaline synthase (Bevan et al., 1983, Nucleic Acids Res. 11(2), 369-385), or the terminator element of a histone gene as described in application EP 0 633 317.

According to a particular embodiment of the invention, the promoter and the terminator element of the chimeric gene according to the invention are both functional in plants.

It also appears to be important for the chimeric gene to additionally comprise a signal peptide or a transit peptide which makes it possible to control and orient the production of the Cry protein specifically in a cellular compartment of the host organism, such as, for example, the cytoplasm, in a particular compartment of the cytoplasm, or the cell membrane or, in the case of plants, in a particular type of cellular compartment, for example the chloroplasts, or in the extracellular matrix.

The transit peptides can be either single or double. The double transit peptides are optionally separated by an intermediate sequence, i.e. they comprise, in the direction of transcription, a sequence encoding a transit peptide of a plant gene encoding an enzyme located in plastids, a portion of sequence of the mature N-terminal portion of a plant gene encoding an enzyme located in plastids, and then a sequence encoding a second transit peptide of a plant gene encoding an enzyme located in plastids. Such double transit peptides are, for example, described in patent application EP 0 508 909.

Signal peptides of use according to the invention which may be mentioned include in particular the signal peptide of the tobacco PR-1α gene described by Cornelissen et al. (1987, Nucleic Acid Res. 15, 6799-6811), in particular when the chimeric gene according to the invention is introduced into plant cells or plants.

The present invention also relates to a vector containing a chimeric gene according to the invention. Such a vector is of use for transforming a host organism and expressing a modified Cry protein according to the invention in said organism. This vector may be a plasmid, a cosmid, a bacteriophage or a virus. In general, the main qualities of this vector should be an ability to maintain itself and to self-replicate in the host organism's cells, in particular by virtue of the presence of an origin of replication, and to express therein a modified Cry protein. The choice of such a vector and also the techniques for inserting the chimeric gene according to the invention therein are widely described in Sambrook et al. (1989, Molecular Cloning: A Laboratory Manual, Nolan C. ed., New York: Cold Spring Harbor Laboratory Press) and are part of the general knowledge of those skilled in the art. The vector used in the present invention may also contain, in addition to the chimeric gene of the invention, a chimeric gene containing a selectable marker. This selectable marker makes it possible to select the host organisms effectively transformed, i.e. those having incorporated the vector. Among the selectable markers which can be used in many host organisms, mention may be made of markers containing genes for resistance to antibiotics, such as that of the hygromycin phosphotransferase gene (Gritz et al., 1983, Gene 25: 179-188). Preferably, the host organism to be transformed is a plant. Among the selectable markers which can be used in plants, mention may be made of markers containing genes for tolerance to herbicides, such as the bar gene (White et al., NAR 18: 1062, 1990) for tolerance to bialaphos, the EPSPS gene (U.S. Pat. No. 5,188,642) for tolerance to glyphosate or else the HPPD gene (WO 96/38567) for tolerance to isoxazoles. Mention may also be made of genes encoding readily identifiable enzymes such as the GUS enzyme, or genes encoding pigments or enzymes which regulate the production of pigments in the transformed cells. Such selectable marker genes are in particular described in patent applications WO 91/02071 and WO 95/06128.

The present invention also relates to host organisms transformed with a vector as described above. The term “host organisms” is intended to mean any type of organism, in particular plants or microorganisms such as bacteria, viruses, fungi or yeast. The term “transformed host organism” is intended to mean a host organism which has incorporated into its genome the chimeric gene of the invention and, consequently, produces a modified Cry protein according to the invention in its tissues. To obtain the host organisms according to the invention, those skilled in the art can use one of the many known methods of transformation. One of these methods consists in bringing the cells to be transformed into contact with polyethylene glycol (PEG) and the vectors of the invention (Chang and Cohen, 1979, Mol. Gen. Genet. 168(1), 111-115; Mercenier and Chassy, 1988, Biochimie 70(4), 503-517). Electroporation is another method, which consists in subjecting the cells or tissues to be transformed and the vectors of the invention to an electric field (Andreason and Evans, 1988, Biotechniques 6(7), 650-660; Shigckawa and Dower, 1989, Aust. J. Biotechnol. 3(1), 56-62). Another method consists in directly injecting the vectors into the host cells or tissues by microinjection (Gordon and Ruddle, 1985, Gene (33(2), 121-136). Advantageously, the “biolistic” method may be used. It consists in bombarding cells or tissues with particles onto which the vectors of the invention are adsorbed (Bruce et al., 1989, Proc. Natl. Acad. Sci. US 86(24), 9692-9696; Klein et al., 1992, Biotechnology 10(3), 286-291; U.S. Pat. No. 4,945,050). Preferably, the transformation of plants will be carried out using bacteria of the Agrobacterium genus, preferably by infecting the cells or tissues of said plants with A. tumefaciens (Knopf, 1979, Subcell. Biochem. 6, 143-173; Shaw et al., 1983, Gene 23(3): 315-330) or A. rhizogenes (Bevan and Chilton, 1982, Annu. Rev. Genet. 16: 357-384; Tepfer and Casse-Delbart, 1987, Microbiol. Sci. 4(1), 24-28). Preferably, the transformation of plant cells with Agrobacterium tumefaciens is carried out according to the protocol described by Ishida et al. (1996, Nat. Biotechnol. 14(6), 745-750).

These various techniques are in particular described in the following patents and patent applications: U.S. Pat. Nos. 4,459,355, 4,536,475, 5,464,763, 5,177,010, 5,187,073, EP 267,159, EP 604 662, EP 672 752, U.S. Pat. Nos. 4,945,050, 5,036,006, 5,100,792, 5,371,014, 5,478,744, 5,179,022, 5,565,346, 5,484,956, 5,508,468, 5,538,877, 5,554,798, 5,489,520, 5,510,318, 5,204,253, 5,405,765, EP 270 615, EP 442 174, EP 486 233, EP 486 234, EP 539 563, EP 674 725, WO 91/02071 and WO 95/06128.

The present invention also relates to a method for producing the modified Cry proteins according to the invention. This method comprises at least the steps of:

a) culturing a transformed host organism according to the invention in a culture medium suitable for the growth and for the multiplication of said organism,

b) extracting the Cry proteins produced by the transformed organism cultured in step (a).

Depending on the host organism chosen to carry out this method and depending on the chimeric gene which it contains, the Cry proteins produced are either produced in the host organism, or are secreted into the culture medium. It ensues that the extraction provided for in step (b) may require a step for destroying the microorganisms, or at least the cells of which they are composed, in order to release the Cry proteins if said proteins are not secreted into the culture medium. The extraction step common to the two possibilities (proteins secreted or not secreted) consists of removal of the host organisms or debris from these organisms by filtration or centrifugation of the culture medium.

According to a particular embodiment, this method for producing the modified Cry proteins can also comprise an additional step (c) of purification of the Cry proteins produced, from the culture medium.

According to a preferred embodiment, the host organism is a microorganism. Preferably, the host organism is a Bacillus thuringiensis bacterium and the culturing performed in step (a) is continued until the sporulation phase of said bacteria.

The present invention also comprises plants transformed with a vector according to the invention, characterized in that they contain a chimeric gene according to the invention stably integrated into their genome, and express a modified Cry protein in their tissues. The invention also extends to the parts of these plants, and the descendants of these plants. The expression “part of these plants” is intended to mean any organ of these plants, whether it is aerial or subterranean. The aerial organs are the stems, the leaves and the flowers. The subterranean organs are mainly the roots, but they can also be tubers. The term “descendants” is intended to mean mainly the seeds containing the embryos derived from the reproduction of these plants with one another. By extension, the term “descendants” applies to all the plants and seeds formed in each new generation derived from crosses between a plant, in particular a plant variety, and a transformed plant according to the invention.

The transformed plants according to the invention may be monocotyledones or dicotyledones. Preferably, these plants are plants of agronomic value. Advantageously, the monocotyledonous plants are wheat, maize and rice. Advantageously, the dicotyledonous plants are rapeseed, soybean, tobacco and cotton.

According to a particular embodiment of the invention, the transformed plants according to the invention contain, in addition to a chimeric gene according to the invention, at least one other gene containing a polynucleotide encoding a protein of interest. Among the polynucleotides encoding a protein of interest, mention may be made of polynucleotides encoding an enzyme for resistance to a herbicide, for example the polynucleotide encoding the bar enzyme (White et al., NAR 18: 1062, 1990) for tolerance to bialaphos, the polynucleotide encoding the EPSPS enzyme (U.S. Pat. No. 5,188,642; WO 97/04103) for tolerance to glyphosate or else the polynucleotide encoding the HPPD enzyme (WO 96/38567) for tolerance to isoxazoles. Also contained in these plants may be polynucleotides for resistance to diseases, for example a polynucleotide encoding the oxalate oxidase enzyme as described in patent application EP 0 531 498 or U.S. Pat. No. 5,866,788, or a polynucleotide encoding an antibacterial and/or antifungal peptide such as those described in patent applications WO 97/30082, WO 99/24594, WO 99/02717, WO 99/53053 and WO 99/91089. Mention may also be made of polynucleotides encoding agronomic characteristics of the plant, in particular a polynucleotide encoding a Δ-6 desaturase enzyme as described in U.S. Pat. Nos. 5,552,306 and 5,614,313, and patent applications WO 98/46763 and WO 98/46764, or a polynucleotide encoding a serine acetyltransferase (SAT) enzyme as described in patent applications WO 00/01833 and PCT/FR 99/03179. The transformed plants according to the invention can also contain a polynucleotide encoding another insecticidal toxin, for example a polynucleotide encoding another Bacillus thuringiensis Cry protein (for example, see international patent application WO 98/40490).

A subject of the present invention is also monoclonal or polyclonal antibodies directed against a modified Cry protein according to the invention, or a fragment thereof. The techniques for producing antibodies are widely described in the general literature and in reference works such as Immunological Techniques Made Easy (1998, 0. Cochet, J. -L. Teillaud, C. Sautès eds., John Wiley & Sons, Chichester). Preferably, the antibodies according to the invention are used in tests, or kits, for detecting the Cry proteins according to the invention.

The examples below make it possible to illustrate the present invention without, however, limiting the scope thereof.

### EXAMPLES

### Example 1

### Creation of a Pepsin Cleavage Site at Amino Acid 164 of the Cry9Ca1 Toxin

A pepsin-specific site is introduced into the Bacillus thuringiensis Cry9Ca1 toxin by substituting the arginine naturally present at position 164 in this toxin with one of the three amino acids recognized by pepsin: leucine, phenylalanine or glutamic acid. Amino acid 164 is present in the inter-α-helix loop linking the α3 and α4 helices of domain I (hereinafter referred to as α3-α4 inter-helix loop)

The native sequence of the α3-α4 inter-helix loop is between aspartic acid 159 and valine 168. The sequence of this loop is as follows: DRNDTRNLSV. This amino acid sequence corresponds to the following DNA sequence extending from base 475 to base 504:

GAT CGA AAT GAT ACA CGA AAT TTA AGT GTT
Asp Arg Asn Asp Thr Arg Asn Leu Ser Val

Codon 164 (CGA) encoding arginine is modified to a codon encoding either leucine or phenylalanine or glutamic acid. The codon possibilities are as follows:

| | |
|---|---|
| Leucine: | TTA, TTG, CTT, CTC, CTA or CTG |
| Phenylalanine: | TTT or TTC |
| Glutamic acid: | GAA or GAG |

The choice of preferential codons in the site-directed mutagenesis depends on the organism in which the modified cry gene must be expressed and therefore varies accordingly. This choice is part of the general knowledge of those skilled in the art, who will adapt the preferential codons as a function of the chosen organism for production. In this example, the chosen organism for expression is the B. thuringiensis bacterium. The codons preferentially used by B. thuringiensis to encode leucine, phenylalanine or glutamic acid are, respectively, TTA (leucine), TTT (phenylalanine) and GAA (glutamic acid).

The modification for expression in Bt can therefore be carried out using the following mutagenesis oligonucleotides (in the oligonucleotides described in the examples below, the codon in upper case letters corresponds to the mutated codon, and the bases and amino acids in bold characters correspond to the bases and amino acids specifically mutated):

Oligonucleotide No. 1:
5′-gat cga aat gat aca TTA aat tta agt gtt gtt-3′
Asp Arg Asn Asp Thr Leu Asn Leu Ser Val Val

Oligonucleotide No. 1 allows the replacement of arginine 164 with a leucine.

Oligonucleotide No. 2:
5′-gat cga aat gat aca TTT aat tta agt gtt gtt-3′
Asp Arg Asn Asp Thr Phe Asn Leu Ser Val Val

Oligonucleotide No. 2 allows replacement of arginine 164 with a phenylalanine.

Oligonucleotide No. 3:
5′-gat cga aat gat aca GAA aat tta agt gtt gtt-3′
Asp Arg Asn Asp Thr Glu Asn Leu Ser Val Val

Oligonucleotide No. 3 allows replacement of arginine 164 with a glutamic acid.

The characteristics of the bacterial strains of Escherichia coli used to modify the sequence of the cry9Ca1 gene are as follows:

JM 109 of genotype recA1 supE44 endA1 hsdR17 gyrA96 relA1 thiD (lac-proAb) F′ (traD36 proAB+lacIq lacZ DM15)

BMH 71-18 mut S of genotype thi, supE, (lac-proAB), (mutS::Tn10)(F′, proAB, lacIqZΔM15).

The plasmid DNA is prepared by minipreparation according to the alkaline lysis technique (Birboim and Doly, 1979). Each bacterial colony is grown in 2 ml of LB medium supplemented with the appropriate antibiotic, overnight at 37° C. with shaking (200 rpm). The culture is then transferred into a microtube and then centrifuged at 13 500 g for 5 min. After removal of the supernatant, the bacteria are resuspended in 100 μl of a solution of 25 mM Tris-HCl, pH 8, and 10 mM EDTA containing RNase A at a final concentration of 100 μg/ml. 200 μl of a 0.2 M NaOH solution containing 1% SDS are added and the suspension is mixed twice by inverting the microtube. 150 μl of a 2.55 M potassium acetate solution, pH 4.5, are added and the suspension is incubated for 5 min in ice. After centrifugation for 15 min at 13 500 g, the supernatant is transferred into a microtube containing 1 ml of cold ethanol. After centrifugation for 30 min at 13 500 g, the supernatant is removed and the pellet is washed with 1 ml of 70% ethanol. The pellet containing the DNA is dried for a few minutes under vacuum and then taken up in 50 μl of sterile distilled water. The samples are then placed at 65° C. for 30 min.

The digestions with restriction endonucleases are carried out for 1 μg of DNA in a final volume of 20 μl in the presence of one tenth of the final volume of 10× buffer recommended by the supplier for each enzyme, and using 5 units of enzyme. The reaction is incubated for 2 to 3 h at the optimal temperature for the enzyme.

Dephosphorylation of the 5′ ends engendered by restriction enzyme is carried out with calf intestine alkaline phosphatase. The reaction is carried out using 5 μl of 10× dephosphorylation buffer (500 mM Tris-HCl, pH 9.3, 10 mM MgCl2, 1 mM ZnCl₂ and 10 mM spermidine) and one unit of enzyme per μg of DNA in a final volume of 50 μl. The reaction is incubated for one hour at 37° C. in the case of overhanging 5′ ends or at 55° C. in the case of blunt ends or 3′ overhanging ends. After dephosphorylation, the enzyme is then inactivated for 30 min at 65° C. and then removed with two volume for volume extractions with a phenol-chloroform-isoamyl alcohol (25-24-1) mixture. The ligations are formed using T4 phage DNA ligase. They are carried out with an amount of vector equal to 100 ng and an insert/vector molar ratio of between 5 and 10. The final volume of the reaction is 30 μl and comprises 3 μl of 10× ligation buffer (300 mM Tris-HCl, pH 7.8, 100 mM MgCl₂, 100 mM DTT and 10 mM ATP) and 3 units of enzyme. The reaction is incubated overnight at 14° C.

The mutagenesis oligonucleotide (oligonucleotide No. 1, oligonucleotide No. 2 and oligonucleotide No. 3) are phosphorylated in the 5′ position in order to allow ligation. 100 pmol of oligonucleotide are incubated for 30 min at 37° C. with 5 units of T4 polynucleotide kinase in a final volume of 25 μl in the presence of 2.5 μl of 10× phosphorylation buffer (700 mM TrisHCl, pH 7.6, 100 mM MgC12 and 50 mM DTT) in the presence of ATP at a final concentration of 1 mM. The enzyme is then inactivated at 70° C. for 10 min.

The site-directed mutagenesis is carried out according to a conventional method described below. Other procedures known to those skilled in the art are described in the literature and give identical results. The site-directed mutagenesis method used is that described by the manufacturer for the use of the Altered Sites II system marketed by the company Promega. A detailed description of the mutagenesis system and of the protocol can be found on the internet site of the company Promega at the address http://www.promega.com. The cry9Ca1 gene is pre-cloned into a phagemide pAlter-1 (Promega) carrying the tetracycline resistance gene and the ampicillin resistance gene containing a point mutation. The DNA fragment to be mutated is pre-cloned into the plasmid pAlter-1. 0.5 pmol of plasmid DNA are denatured by adding 2 μl of 2 M NaOH, 2 mM EDTA in a final volume of 20 μl and incubating for 5 min at ambient temperature. 2 μl of 2 M ammonium acetate, pH 4.6, and 75 μl of ethanol are added and the mixture is incubated at −70° C. for 30 min. After centrifugation at 14 000 g for 15 min at 4° C., the pellet is then rinsed with 200 μl of 70% ethanol and recentrifuged at 14 000 g for 15 min at 4° C. The denatured DNA pellet is then dried under vacuum and resuspended in 100 μl of sterile distilled water. 10 μl of denatured DNA, i.e. 0.05 pmol, are mixed with 0.25 pmol of phosphorylated ampicillin-resistance gene repair oligonucleotide, 0.25 pmol of tetracycline-resistance gene destruction oligonucleotide and 1.25 pmol of phosphorylated mutagenesis oligonucleotide (oligonucleotide No. 1, No. 2 or No 3) in the presence of hybridization buffer (20 mM Tris-HCl, pH 7.5, 10 mM MgCl2, 50 mM NaCl) and incubated at 75° C. for 5 min, and then slowly cooled to ambient temperature. 5 μl of sterile distilled water, 3 μl of 10× synthesis buffer (100 mM Tris-HCl, pH 7.5, 20 mM DTT, 10 mM ATP, 5 mM dNTP), 10 units of T4 DNA polymerase and 3 units of T4 DNA ligase are added and the reaction is incubated for 90 min at 37° C. 200 μl of competent E. coli BMH 71-18 bacteria are then incubated in the presence of 1.5 μl of the preceding reaction, in ice for 30 min. A heat shock is then performed by placing the bacteria at 42° C. for 50 sec and then in ice for 2 min. 900 μl of LB medium are then added and the suspension is incubated at 37° C. for one hour with shaking. 4 ml of LB medium supplemented with ampicillin at the final concentration of 100 μg/ml are then added and the culture is incubated overnight at 37° C. with shaking. A minipreparation of plasmid DNA is prepared from the 4 ml of culture according to the plasmid DNA extraction protocol described above. 200 μl of competent E. coli JM109 bacteria are then incubated in the presence of 1 ng of plasmid DNA, in ice for 30 min. A heat shock is then performed by placing the bacteria at 42° C. for 50 sec, and then in ice for 2 min. 900 μl of LB medium are then added and the suspension is incubated at 37° C. overnight with shaking. 100 μl of bacterial suspension are then plated out on a Petri dish containing solid LB medium supplemented with ampicillin at the final concentration of 100 μg/ml. The recombinants obtained are screened in order to find the clone of interest. This search is carried out by isolating the plasmid DNA of several colonies by the minipreparation technique described above, and then by sequencing this DNA. The recombinants are then selected using medium supplemented with tetracycline at the final concentration of 12.5 μg/ml. The correctness of the desired mutation and the verification of the lack of undesirable mutations are controlled by sequencing the DNA after site-directed mutagenesis. DNA samples for the sequencing are purified with the Wizard Plus SV Minipreps DNA Purification System (Promega) according to the procedure recommended by the supplier, and the sequencing is carried out on an ABI 377 automatic sequencer (Perkin-Elmer) using sequencing reactions carried out according to the chain termination method (Sanger et al., 1977), by PCR using the ABI PRISM BigDye terminator Cycle Sequencing Kit system. For carrying out the sequencing reactions and the automatic analysis of the samples, the procedures used are those recommended by the supplier (Applied Biosystems).

### Example 2

### Creation of Pepsin Cleavage Sites in the α3-α4 Inter-helix Loop of the Cry9Ca1 Toxin

Pepsin-specific sites are introduced into the α3-α4 inter-helix loop of the Cry9Ca1 toxin by substituting at least one amino acid of this inter-helix loop with an amino acid recognized by pepsin, namely leucine, phenylalanine and glutamic acid. Codons encoding these three amino acids will therefore be created in place of the codons naturally present in the region extending from base 475 to base 504. The codon possibilities for these three amino acids are described in Example 1.

As in Example 1, the selected organism for producing the modified Cry protein is the B. thuringiensis bacterium, and the choice of the replacement codons is therefore identical to that of Example 1. In addition, if another organism for production is selected, those skilled in the art will be able to adjust the preferential codons as a function of the organism for production selected.

Various alternative sequences for the α3-α4 inter-helix loop are possible, each having a variable number of leucine, phenylalanine or glutamic acid residues. Some of these possibilities are given in Table 1. The possibilities for modification of the α3-α-4 inter-helix loop are not limited to those given in Table 1 below. The aim of the list given in Table 1 is to illustrate some of the possibilities for modification without limiting the scope of the invention to these illustrations. Those skilled in the art, being aware of the codons specific for each amino acid according to the organisms, will be able to adapt the teaching described in this example to all the possibilities for modifying the α3-α4 inter-helix loop, in particular to those which are not described in Table 1.

TABLE 1
Examples of possible modifications of the α3-α4
inter-helix loop of the Cry9Ca1 toxin

| | | |
|---|---|---|
| | Amino acid | |
| Protein | sequence | Nucleotide sequence |

| | | |
|---|---|---|
| CryCa1 | DRNDTRNLSV | gat cga aat gat aca cga aat tta agt gtt |
| | | Asp Arg Asn Asp Thr Arg Asn Leu Ser Val |
| Mutant No. 1 | ELNEFLNSV | gaA TTA aat gaA TTT TTa aat tta agt gtt |
| | | Glu Leu Asn Glu Phe Leu Asn Leu Ser Val |
| Mutant No. 2 | ELNELLNLSV | gaA TTA aat gaA TTa TTa aat tta agt gtt |
| | | Glu Leu Asn Glu Leu Leu Asn Leu Ser Val |
| Mutant No. 3 | ELLEFLLLSV | gaA TTa TTA gaA TTT TTa TTA tta agt gtt |
| | | Glu Leu Leu Glu Phe Leu Leu Leu Ser Val |
| Mutant No. 4 | ELLELLLLSV | gaA TTa TTA gaA TTa TTa TTA tta agt gtt |
| | | Glu Leu Leu Glu Leu Leu Leu Leu Ser Val |
| Mutant No. 5 | ELLEELLLSV | gaA TTa TTA gaA GAa TTa TTa tta agt gtt |
| | | Glu Leu Leu Glu Glu Leu Leu Leu Ser Val |
| Mutant No. 6 | ERLEFLLLSV | gaA cga TTA gaA TTT TTa TTA tta agt gtt |
| | | Glu Arg Leu Glu Phe Leu Leu Leu Ser Val |
| Mutant No. 7 | ERLELLLLSV | gaA cga TTA gaA TTa TTa TTA tta agt gtt |
| | | Glu Arg Leu Glu Leu Leu Leu Leu Ser Val |
| Mutant No. 8 | ERLEELLLSV | gaA TTa GAA gaA TTa TTa TTA tta agt gtt |
| | | Glu Leu Glu Glu Leu Leu Leu Leu Ser Val |
| Mutant No. 9 | ELLEEEELSV | gaA TTa TTA gaA GAa GAa GAA tta agt gtt |
| | | Glu Leu Leu Glu Glu Glu Glu Leu Ser Val |

The substitution of several amino acids within the α3-α4 inter-helix loop requires, for each of the mutants, the successive use of several mutagenesis oligonucleotides. The mutagenesis oligonucleotides required to create the examples of mutants given in Table 1 are presented below (numbered from 4 to 20).

| | |
|---|---|
| Oligonucleotide No. 4: | cga aat gat aca cga TTA tta agt gtt gtt cgt |
| | Arg Asn Asp Thr Arg Leu Leu Ser Val Val Arg |
| Oligonucleotide No. 5: | cga aat gat aca cga GAA tta agt gtt gtt cgt |
| | Arg Asn Asp Thr Arg Glu Leu Ser Val Val Arg |
| Oligonucleotide No. 6: | ttg gct gat cga aat gaA TTT TTa aat tta agt gtt gtt |
| | Leu Ala Asp Arg Asn Glu Phe Leu Asn Leu Ser Val Val |
| Oligonucleotide No. 7: | ttg gct gat cga aat gaA TTT TTa tta tta agt gtt gtt |
| | Leu Ala Asp Arg Asn Glu Phe Leu Leu Leu Ser Val Val |
| Oligonucleotide No. 8: | ttg gct gat cga aat gaA TTa TTa aat tta agt gtt gtt |
| | Leu Ala Asp Arg Asn Glu Leu Leu Asn Leu Ser Val Val |
| Oligonucleotide No. 9: | ttg gct gat cga aat gaA TTa TTa tta tta agt gtt gtt |
| | Leu Ala Asp Arg Asn Glu Leu Leu Leu Leu Ser Val Val |
| Oligonucleotide No. 10: | ttg gct gat cga aat gaA GAa GAa gaa tta agt gtt gtt |
| | Leu Ala Asp Arg Asn Glu Glu Glu Glu Leu Ser Val Val |
| Oligonucleotide No. 11: | ttg gct gat cga aat gaA GAa TTa tta tta agt gtt gtt |
| | Leu Ala Asp Arg Asn Glu Glu Leu Leu Leu Ser Val Val |
| Oligonucleotide No. 12: | caa aat tgg ttg gct gaA TTa aat gaa tta tta aat |
| | Gln Asn Trp Leu Ala Glu Leu Asn Glu Leu Leu Asn |
| Oligonucieotide No. 13: | caa aat tgg ttg gct gaA TTa aat gaa ttt tta aat |
| | Gln Asn Trp Leu Ala Glu Leu Asn Glu Phe Leu Asn |
| Oligonucleotide No. 14: | caa aat tgg ttg gct gaA TTa TTA gaa ttt tta tta tta |
| | Gln Asn Trp Leu Ala Glu Leu Leu Glu Phe Leu Leu Leu |
| Oligonucleotide No. 15: | caa aat tgg ttg gct gaA TTa TTA gaa tta tta tta tta |
| | Gln Asn Trp Leu Ala Glu Leu Leu Glu Leu Leu Leu Leu |
| Oligonucleotide No. 16: | caa aat tgg ttg gct gaA TTa TTA gaa gaa tta tta tta |
| | Gln Asn Trp Leu Ala Glu Leu Leu Glu Glu Leu Leu Leu |
| Oligonucleotide No. 17: | caa aat tgg ttg gct gaA cga TTA gaa ttt tta tta tta |
| | Gln Asn Trp Leu Ala Glu Arg Leu Glu Phe Leu Leu Leu |
| Oligonucleotide No. 18: | caa aat tgg ttg gct gaA cga TTA gaa tta tta tta tta |
| | Gln Asn Trp Leu Ala Glu Arg Leu Glu Leu Leu Leu Leu |
| Oligonucleotide No. 19: | caa aat tgg ttg gct gaA TTa gaA gaa tta tta tta tta |
| | Gln Asn Trp Leu Ala Glu Leu Glu Glu Leu Leu Leu Leu |
| Oligonucleotide No. 20: | caa aat tgg ttg gct gaA TTa TTA gaa gaa gaa gaa tta |

The successive site-directed mutagenesis procedure is similar to the procedure described in Example 1. The difference lies in the combination of oligonucleotides. For each of the examples of mutants described in Table 1, the successive combinations of oligonucleotides are described below.

Mutant No. 1: The creation of mutant No. 1 requires two successive series of site-directed mutagenesis according to the protocol described in Example 1, using oligonucleotide No. 6 in the first mutagenesis and oligonucleotide No. 13 in the second. Oligonucleotide No. 13 is defined to recognize the modifications introduced during the first mutagenesis with oligonucleotide No. 6.

Mutant No. 2: The creation of mutant No. 2 requires two successive series of site-directed mutagenesis according to the protocol described in Example 1, using oligonucleotide No. 8 in the first mutagenesis and oligonucleotide No. 12 in the second. Oligonucleotide No. 12 is defined to recognize the modifications introduced during the first mutagenesis with oligonucleotide No. 8.

Mutant No. 3: The creation of mutant No. 3 requires three successive series of site-directed mutagenesis according to the protocol described in Example 1, using oligonucleotide No. 4 in the first mutagenesis, oligonucleotide No. 7 in the second and oligonucleotide No. 14 in the third. Oligonucleotide No. 7 is defined to recognize the modifications introduced during the first mutagenesis with oligonucleotide No. 4 and oligonucleotide No. 14 is defined to recognize the modifications introduced during the first two mutageneses with oligonucleotides No. 4 and No. 7.

Mutant No. 4: The creation of mutant No. 4 requires three successive series of site-directed mutagenesis according to the protocol described in Example 1, using oligonucleotide No. 4 in the first mutagenesis, oligonucleotide No. 9 in the second and oligonucleotide No. 15 in the third. Oligonucleotide No. 9 is defined to recognize the modifications introduced during the first mutagenesis with oligonucleotide No. 4 and oligonucleotide No. 15 is defined to recognize the modifications introduced during the first two mutageneses with oligonucleotides No. 4 and No. 9.

Mutant No. 5: The creation of mutant No. 5 requires three successive series of site-directed mutagenesis according to the protocol described in Example 1, using oligonucleotide No. 4 in the first mutagenesis, oligonucleotide No. 11 in the second and oligonucleotide No. 16 in the third. Oligonucleotide No. 11 is defined to recognize the modifications introduced during the first mutagenesis with oligonucleotide No. 4 and oligonucleotide No. 16 is defined to recognize the modifications introduced during the first two mutageneses with oligonucleotides No. 4 and No. 11.

Mutant No. 6: The creation of mutant No. 6 requires three successive series of site-directed mutagenesis according to the protocol described in Example 1, using oligonucleotide No. 4 in the first mutagenesis, oligonucleotide No. 7 in the second and oligonucleotide No. 17 in the third. Oligonucleotide No. 7 is defined to recognize the modifications introduced during the first mutagenesis with oligonucleotide No. 4 and oligonucleotide No. 17 is defined to recognize the modifications introduced during the first two mutageneses with oligonucleotides No. 4 and No. 7.

Mutant No. 7: The creation of mutant No. 7 requires three successive series of site-directed mutagenesis according to the protocol described in Example 1, using oligonucleotide No. 4 in the first mutagenesis, oligonucleotide No. 9 in the second and oligonucleotide No. 18 in the third. Oligonucleotide No. 9 is defined to recognize the modifications introduced during the first mutagenesis with oligonucleotide No. 4 and oligonucleotide No. 18 is defined to recognize the modifications introduced during the first two mutageneses with oligonucleotides No. 4 and No. 9.

Mutant No. 8: The creation of mutant No. 8 requires three successive series of site-directed mutagenesis according to the protocol described in Example 1, using oligonucleotide No. 4 in the first mutagenesis, oligonucleotide No. 9 in the second and oligonucleotide No. 19 in the third. Oligonucleotide No. 9 is defined to recognize the modifications introduced during the first mutagenesis with oligonucleotide No. 4 and oligonucleotide No. 19 is defined to recognize the modifications introduced during the first two mutageneses with oligonucleotides No. 4 and No. 9.

Mutant No. 9: The creation of mutant No. 9 requires three successive series of site-directed mutagenesis according to the protocol described in Example 1, using oligonucleotide No. 5 in the first mutagenesis, oligonucleotide No. 10 in the second and oligonucleotide No. 20 in the third. Oligonucleotide No. 10 is defined to recognize the modifications introduced during the first mutagenesis with oligonucleotide No. 5 and oligonucleotide No. 20 is defined to recognize the modifications introduced during the first two mutageneses with oligonucleotides No. 5 and No. 10.

According to this protocol, the oligonucleotides are divided up into three categories, 1st series oligonucleotides, 2nd series oligonucleotides and 3rd series oligonucleotides. This division is as follows:

| | |
|---|---|
| 1st series oligonucleotides: | oligonucleotides No. 4, 5, 6 and 8 |
| 2nd series oligonucleotides: | oligonucleotides No. 7, 9, 10, 11, |
| | 12 and 13 |
| 3rd series oligonucleotides: | oligonucleotides No. 14, 15, 16, 17, |
| | 18, 19 and 20. |

The complete protocol for producing these mutants is identical to that described in Example 1. This protocol is common to each of the series of mutageneses, only the mutagenesis oligonucleotide and the oligonucleotide for inhibition/restoration of the resistance to the antibiotic change. The passing onto the following mutation takes place after screening of the clone of interest which has integrated the preceding mutation. If this step is the final step of the first series or of the second series of mutagenesis, the material derived from this series of experiments is re-used as initial material for, respectively, the 2nd or 3rd series of mutagenesis using, respectively, the 2nd or 3rd series oligonucleotides. A second cycle of mutagenesis can then be carried out using the plasmid DNA obtained as DNA matrix and also the oligonucleotide for repair of the tetracycline resistance gene and the oligonucleotide for destruction of the ampicillin resistance gene and a 2nd series mutagenesis oligonucleotide. The recombinants are then selected using medium supplemented with tetracycline at the final concentration of 12.5 μg/ml. A third cycle of mutagenesis can be carried out using the plasmid DNA obtained at the end of the second cycle of mutagenesis as DNA matrix and also the oligonucleotide for repair of the ampicillin resistance gene and the oligonucleotide for destruction of the tetracycline resistance gene and a 3rd series mutagenesis oligonucleotide. The recombinants are then selected using medium supplemented with ampicillin at the final concentration of 100 μg/ml. After all the series of mutagenesis required to produce a mutant have been carried out, the steps for controlling the mutations are carried out as described in Example 1.

### Example 3

### Creation of Pepsin Cleavage Sites in the α4-α5, α5-α6 and α6-α7 Inter-helix Loops of the Cry9Ca1 Toxin

The positions of the native sequences of the α4-α5, (α5-α6 and α6-α7 inter-helix loops of the Cry9Ca1 toxin are given in Table 2 below. The nucleotide sequences and the corresponding positions in the cry9Ca1 gene are given in Table 3.

TABLE 2
Position and sequences of the α4-α5, α5-α6 and α6-α7
inter-helix loops of the Cry9Ca1 toxin

| Loop | Sequence | Position |
|---|---|---|
| Loop α4-α5 | FAVNGQQVPLL | Phenylalanine 187 to leucine 197 |
| Loop α5-α6 | LFGEGWGF | Leucine 216 to phenylalanine 223 |
| Loop α6-α7 | LRGTN | Leucine 257 to asparagine 261 |

TABLE 3
Position and sequences of cry9Ca1 gene encoding the α4-α5,
α5-α6 and α6-α7 inter-helix loops

| Loop | Sequence | Position |
|---|---|---|
| Loop α4-α5 | TTT GCA GTA AAT GGA CAG CAG GTT CCA TTA CTG | 559-591 |
| Loop α5-α6 | CTT TTT GGA GAA GGA TGG GGA TTC | 646-669 |
| Loop α6-α7 | TTA AGA GGA ACA AAT | 769-783 |

The superposition of the nucleotide and amino acid sequences are as follows:

| | |
|---|---|
| Loop | TTT GCA GTA AAT GGA CAG CAG GTT CCA TTA CTG |
| α4-α5: | Phe Ala Val Asn Gly Gln Gln Val Pro Leu Leu |
| Loop | CTT TTT GGA GAA GGA TGG GGA TTC |
| α5-α6 | Leu Phe Gly Glu Gly Trp Gly Phe |
| Loop | TTA AGA GGA ACA AAT |
| α6-α7 | Leu Arg Gly Thr Asn |

Pepsin-specific sites are introduced into the α4-α5, α5-α6 or α6-α7 inter-helix loops of the Cry9Ca1 toxin by substituting at least one amino acid of these inter-helix loops with an amino acid recognized by pepsin, namely leucine, phenylalanine and glutamic acid. Codons encoding these three amino acids will therefore be created in place of the codons naturally present in the region extending from bases 559 to 591 (α4-α5 inter-helix loop), 646 to 669 (α5-α6 inter-helix loop), and 769 to 783 (α6-α7 inter-helix loop). The codon possibilities for these three amino acids are described in Example 1.

As in Example 1, the chosen organism for producing the modified Cry protein is the B. thuringiensis bacterium, and the choice of the replacement codons is therefore identical to that of Example 1. In addition, if another organism for production is chosen, those skilled in the art will be able to adjust the preferential codons as a function of the chosen organism for production.

Various alternative sequences for the α4-α5, α5-α6 and α6-α7 inter-helix loops are possible, each one having a variable number of leucine, phenylalanine or glutamic acid residues. Several of these various possibilities are given in Tables 4, 5 and 6. The possibilities for modification of the α4-α5, α5-α6 and α6-α7 inter-helix loops are not limited to those given in Tables 4, 5 and 6 below. The aim of the list given in Tables 4, 5 and 6 is to illustrate some of the possibilities for modification without limiting the scope of the invention to these illustrations. Those skilled in the art, being aware of the codons specific for each amino acid according to the organisms, will be able to adapt the teaching described in this example to all the possibilities for modifying the α4-α5, α5-α6 and α6-α7 inter-helix loops, in particular to those which are not described in Tables 4, 5 and 6.

TABLE 4
Examples of possible modifications of the α4-α5
inter-helix loop of the Cry9Ca1 toxin

| Protein | Amino acid sequence | Nucleotide sequence |
|---|---|---|
| CryCa1 | FAVNGQVPLL | ttt gca tga aat gga cag cag gtt cca tta ctg |
| | | Phe Ala Val Asn Gly Gln Gln Val Pro Leu leu |
| Mutant No. 10 | FLLNLFFLPLL | ttt TTa Tta aat TTa TTT TTT TtA cca tta ctg |
| | | Phe Leu leu Asn Leu Phe Phe Leu Pro Leu leu |
| Mutant No. 11 | FLLNLEELPLL | ttt TTa Tta aat TTa GaA GaA TtA cca tta ctg |
| | | Phe Leu leu Asn Leu Glu Glu Leu Pro Leu Leu |
| Mutant No. 12 | FEENLEELPLL | ttt GAa GAa aat TTA GaA GaA TtA cca tta ctg |
| | | Phe Glu Glu Asn Leu Glu Glu Leu Pro Leu leu |
| Mutant No. 13 | FEENFLLFPLL | ttt GAa GAa aat TTT TTA TTA Ttt cca tta ctg |
| | | Phe Glu Glu Asn Phe leu Leu Phe Pro Leu leu |
| Mutant No. 14 | FEENFEEFPLL | ttt GAa GAa aat TTT GaA GaA Ttt cca tta ctg |
| | | Phe Glu Glu Asn Phe Glu Glu Phe Pro Leu leu |
| Mutant No. 15 | FLLNFEEFPLL | ttt TTa TTa aat TTT GaA GaA Ttt cca tta ctg |
| | | Phe Leu leu Asn Phe Glu Glu Phe Pro Leu leu |
| Mutant No. 16 | FLLNEFFEPLL | ttt TTa TTa aat GAa TTT TTT gAA cca tta ctg |
| | | Phe Leu leu Asn Glu Phe Phe Glu Pro Leu leu |

TABLE 5
Examples of possible modifications
of the α5-α6 inter-helix loop of the Cry9Cal toxin

| | Amino acid | |
|---|---|---|
| Protein | sequence | Nucleotide sequence |
| Cry9Ca1 | LFGEGWGF | ctt ttt gga gaa gga tgg gga ttc |
| | | Leu Phe Gly Glu Gly Trp Gly Phe |
| Mutant | LFLELFLF | ctt ttt TTa gaa TTa tTT TTa ttc |
| No. 17 | | Leu Phe Leu Gly Leu Phe Leu Phe |
| Mutant | LFLLLFLF | ctt ttt TTa TTa TTa tTT TTa ttc |
| No. 18 | | Leu Phe Leu Leu Leu Phe Leu Phe |
| Mutant | LFLEEFEL | ctt ttt TTa gaa gAa tTT gAa TTA |
| No. 19 | | Leu Phe Leu Glu Glu Phe Glu Leu |
| Mutant | LFEEEFEL | ctt ttt gAa gaa gAa tTT gAa TTA |
| No. 20 | | Leu Phe Glu Glu Glu Phe Glu Leu |
| Mutant | LFEEEFEE | ctt ttt gAa gaa TTa tTT gAa GAA |
| No. 21 | | Leu Phe Glu Glu Glu Phe Glu Glu |

TABLE 6
Examples of possible modifications
of the α6-α7 inter-helix loop of the Cry9Ca1 toxin

| | Amino acid | |
|---|---|---|
| Protein | sequence | Nucleotide sequence |
| Cry9Ca1 | LRGTN | tta aga gga aca aat |
| | | Leu Arg Gly thr Asn |
| Mutant No. 22 | LLELN | tta TTa gAa TTa aat |
| | | Leu Leu Glu Leu Asn |
| Mutant No. 23 | LLFLN | tta TTa TTT TTa aat |
| | | Leu Leu Phe Leu Asn |
| Mutant No. 24 | LELLN | tta GAa TTa TTa aat |
| | | Leu Glu Leu Leu Asn |
| Mutant No. 25 | LLFFN | tta TTa TTT TTT aat |
| | | Leu Leu Phe Phe Asn |
| Mutant No. 26 | LEELN | tta GAa GAa TTa aat |
| | | Leu Glu Glu Leu Asn |
| Mutant No. 27 | LEFLN | tta GAa TTT TTa aat |
| | | Leu Glu Phe Leu Asn |
| Mutant No. 28 | LEFEN | tta GAa TTT GAa aat |
| | | Leu Glu Phe Glu Asn |
| Mutant No. 29 | LEEEN | tta GAa gAa GAa aat |
| | | Leu Glu Glu Glu Asn |

### 3-1-Creation of Pepsin Cleavage Sites in the α4-α5 Inter-helix Loop

The substitution of several amino acids within the α4-α5 inter-helix loop requires, for each of the mutants, the successive use of several mutagenesis oligonucleotides. The mutagenesis oligonucleotides required to create the examples of mutants given in Table 4 are presented below (numbered from 21 to 34).

| | |
|---|---|
| Oligonucleotide No. 21: | gct att cca ttg ttt TTa Tta aat gga cag cag gtt |
| | Ala Ile Pro Leu Phe Leu Ile Asn Gly Gln Gln Val |
| Oligonucleotide No. 22: | gct att cca ttg ttt GAa GAa aat gga cag cag gtt |
| | Ala Ile Pro Leu Phe Glu Glu Asn Gly Gln Gln Val |
| Oligonucleotide No. 23: | tta tta aat gga cag cag TtA cca tta ctg tca gta |
| | Leu leu Ann Gly Gln Gln Leu Pro Leu Leu Ser Val |
| Oligonucleotide No. 24: | tta tta aat gga cag cag Ttt cca tta ctg tca gta |
| | Leu leu Asn Gly Gln Gln Phe Pro Leu Leu Ser Val |
| Oiigonucleotide No. 25: | tta tta aat gga cag cag gAA cca tta ctg tca gta |
| | Leu leu Asn Gly Gln Gln Glu Pro Leu Leu Ser Val |
| Oligonucleotide No. 26: | gaa gaa aat gga cag cag TtA caa tta ctg tca gta |
| | Glu Glu Asn Gly Gln Gln Leu Pro Lou Leu Ser Val |
| Oligonucleotide No. 27: | gaa gaa aat gga cag cag Ttt cca tta ctg tca gta |
| | Glu Glu Asn Gly Gln Gln Phe Pro Leu Leu Ser Val |
| Oligonucleotide No. 28: | cca ttg ttt tta tta aat TTa TTT TTT tta cca tta ctg tca gta |
| | Pro Lou Phe Lou Leu Asn Leu Phe Phe Leu Pro Leu Leu Ser Val |
| Oligonucleotide No. 29: | cca ttg ttt tta tta aat TTa GaA GaA tta cca tta ctg tca gta |
| | Pro Leu Phe Leu Leu Asn Leu Glu Glu Leu Pro Leu Leu Ser Val |
| Oligonucleotide No. 30: | cca ttg ttt gaa gaa aat TTa GaA GaA tta cca tta ctg tca gta |
| | Pro Leu Phe Glu Glu Asn Leu Glu Glu Leu Pro Leu Leu Ser Val |
| Oligonucleotide No. 31: | cca ttg ttt gaa gaa aat TTT TTA TTA ttt cca tta ctg tca gta |
| | Pro Leu Phe Glu Glu Asn Phe Leu Leu Phe Pro Leu Leu Ser Val |
| Oligonucleotide No. 32: | cca ttg ttt gaa gaa aat TTT GaA GaA ttt cca tta ctg tca gta |
| | Pro Leu Phe Glu Glu Asn Phe Glu Glu Phe Pro Leu Leu Ser Val |
| Oligonucleotide No. 33: | cca ttg ttt tta tta aat TTT GaA GaA ttt cca tta ctg tca gta |
| | Pro Leu Phe Leu Leu Asn Phe Glu Glu Phe Pro Leu Leu Ser Val |
| Oligonucleotide No. 34: | cca ttg ttt tta tta sat GAa TTT TTT gaa cca tta ctg tca gta |
| | Pro Leu Phe Leu Leu Asn Glu Phe Phe Glu Pro Leu Leu Ser Val |

The successive site-directed mutagenesis procedure is similar to the procedure described in Example 2. The only difference lies in the combination of oligonucleotides. For each of the mutants described in Table 4, the successive combinations of oligonucleotides are described below.

Mutant No. 10: The creation of mutant No. 10 requires three successive series of site-directed mutageneses according to the protocol described below, using oligonucleotide No. 21 in the first mutagenesis, oligonucleotide No. 23 in the second and oligonucleotide No 28 in the third. Oligonucleotide No. 23 is defined to recognize the modifications introduced during the first mutagenesis with oligonucleotide No. 21 and oligonucleotide No. 28 is defined to recognize the modifications introduced during the first two mutageneses with oligonucleotides No. 21 and 23.

Mutant No. 11: The creation of mutant No. 11 requires three successive series of site-directed mutageneses according to the protocol described below, using oligonucleotide No. 21 in the first mutagenesis, oligonucleotide No. 23 in the second and oligonucleotide No 29 in the third. Oligonucleotide No. 23 is defined to recognize the modifications introduced during the first mutagenesis with oligonucleotide No. 21 and oligonucleotide No. 29 is defined to recognize the modifications introduced during the first two mutageneses with oligonucleotides No. 21 and 23.

Mutant No. 12: The creation of mutant No. 12 requires three successive series of site-directed mutageneses according to the protocol described below, using oligonucleotide No. 22 in the first mutagenesis, oligonucleotide No. 26 in the second and oligonucleotide No 30 in the third. Oligonucleotide No. 26 is defined to recognize the modifications introduced during the first mutagenesis with oligonucleotide No. 22 and oligonucleotide No. 30 is defined to recognize the modifications introduced during the first two mutageneses with oligonucleotides No. 22 and 26.

Mutant No. 13: The creation of mutant No. 13 requires three successive series of site-directed mutageneses according to the protocol described below, using oligonucleotide No. 22 in the first mutagenesis, oligonucleotide No. 27 in the second and oligonucleotide No 31 in the third. Oligonucleotide No. 27 is defined to recognize the modifications introduced during the first mutagenesis with oligonucleotide No. 22 and oligonucleotide No. 31 is defined to recognize the modifications introduced during the first two mutageneses with oligonucleotides No. 22 and 27.

Mutant No. 14: The creation of mutant No. 14 requires three successive series of site-directed mutageneses according to the protocol described below, using oligonucleotide No. 22 in the first mutagenesis, oligonucleotide No. 27 in the second and oligonucleotide No 32 in the third. Oligonucleotide No. 27 is defined to recognize the modifications introduced during the first mutagenesis with oligonucleotide No. 22 and oligonucleotide No. 32 is defined to recognize the modifications introduced during the first two mutageneses with oligonucleotides No. 22 and 27.

Mutant No. 15: The creation of mutant No. 15 requires three successive series of site-directed mutageneses according to the protocol described below, using oligonucleotide No. 21 in the first mutagenesis, oligonucleotide No. 24 in the second and oligonucleotide No 33 in the third. Oligonucleotide No. 24 is defined to recognize the modifications introduced during the first mutagenesis with oligonucleotide No. 21 and oligonucleotide No. 33 is defined to recognize the modifications introduced during the first two mutageneses with oligonucleotides No. 21 and 24.

Mutant No. 16: The creation of mutant No. 16 requires three successive series of site-directed mutageneses according to the protocol described below, using oligonucleotide No. 21 in the first mutagenesis, oligonucleotide No. 25 in the second and oligonucleotide No 34 in the third. Oligonucleotide No. 25 is defined to recognize the modifications introduced during the first mutagenesis with oligonucleotide No. 21 and oligonucleotide No. 34 is defined to recognize the modifications introduced during the first two mutageneses with oligonucleotides No. 21 and 25.

According to this protocol, the oligonucleotides intended to create the mutants No. 10 to No. 16 described in Table 4 are divided up into three categories, 1st series oligonucleotides, 2nd series oligonucleotides and 3rd series oligonucleotides. This division is as follows:

| | |
|---|---|
| 1st series oligonucleotides: | oligonucleotides No. 21 and 22 |
| 2nd series oligonucleotides: | oligonucleotides No. 23, 24, 25, 26 and 27 |
| 3rd series oligonucleotides: | oligonucleotides No. 28, 29, 30, 31, 32, 33 |
| | and 34. |

### 3-2-Creation of Pepsin Cleavage Sites in the α5-α6 Inter-helix Loop

The substitution of several amino acids within the α5-α6 inter-helix loop requires, for each of the mutants, the successive use of several mutagenesis oligonucleotides. The mutagenesis oligonucleotides required to create the examples of mutants given in Table 5 are presented below (numbered from 35 to 44).

| | |
|---|---|
| Oligonucleotide No. 35: | gat gca tct ctt ttt TTa gaa gga tgg gga ttc |
| | Asp Ala Ser Leu Phe Leu Glu Gly Trp Gly Phe |
| Ollgonucleotide No. 36: | gat gca tct ctt ttt TTa TTa gga tgg gga ttc aca |
| | Asp Ala Ser Leu Phe Leu Leu Gly Trp Gly Phe Thr |
| Oligonucleotide No. 37: | gat gca tct ctt ttt gAa gaa gga tgg gga ttc |
| | Asp Ala Ser Leu Phe Glu Glu Gly Trp Gly Phe |
| Oligonucleotide No. 38: | tta gaa gga tgg gga TTa aca sag ggg gaa att |
| | Leu Glu Gly Trp Gly Leu Thr Gln Gly Glu Ile |
| Oligonucleotide No. 39: | gga gaa gga tgg gga GAA aca sag ggg gaa att |
| | Gly Glu Gly Trp Gay Glu Thr Gln Gay Glu Ile |
| Oligonucleotide No. 40: | gca tct ctt ttt tta gaa TTa tTT TTa ttc aca cag gqg gaa att |
| | Ala Ser Leu Phe Leu Glu Leu Phe Leu Phe Thr Gln Gly Glu Ile |
| Oligonucleotide No. 41: | gca tot ctt ttt tta tta TTa tTT TTa ttc aca cag ggg gaa att |
| | Ala Ser Leu Phe Leu Leu Leu Phe Leu Phe Thr Gln Gly Glu Ile |
| Oligonucleotide No. 42: | gca tct ctt ttt tta gaa TTa tTT TTa ttc aca cag ggg gaa att |
| | Ala Ser Leu Phe Leu Glu Glu Phe Gln Leu Thr Gln Gly Glu Ile |
| Qligonucleotide No. 43: | gca tct ctt ttt gaa gaa TTa tTT TTa ttc aca cag ggg gaa att |
| | Ala Ser Leu Phe Glu Glu Glu Phe Glu Leu Thr Gln Gly Glu Ile |
| Oligonucleotide No. 44: | gca tct ctt ttt gaa gaa TTA tTT TTa gaa aca cag ggg gaa att |
| | Ala Ser Leu Phe Glu Glu Glu Phe Glu Glu Thr Gln Gly Glu Ile |

The successive site-directed mutagenesis procedure is similar to the procedure described in Example 2. The only difference lies in the combination of oligonucleotides. For each of the mutants described in Table 5, the successive combination of oligonucleotides are described below.

Mutant No. 17: The creation of mutant No. 17 requires two successive series of site-directed mutageneses according to the protocol described below, using oligonucleotide No. 35 in the first mutagenesis and oligonucleotide No. 40 in the second. Oligonucleotide No. 40 is defined to recognize the modifications introduced during the first mutagenesis with oligonucleotide No. 35.

Mutant No. 18: The creation of mutant No. 18 requires two successive series of site-directed mutageneses according to the protocol described below, using oligonucleotide No. 36 in the first mutagenesis and oligonucleotide No. 41 in the second. Oligonucleotide No. 41 is defined to recognize the modifications introduced during the first mutagenesis with oligonucleotide No. 36.

Mutant No. 19: The creation of mutant No. 19 requires three successive series of site-directed mutageneses according to the protocol below, using oligonucleotide No. 35 in the first mutagenesis, oligonucleotide No. 38 in the second and oligonucleotide No. 42 in the third. Oligonucleotide No. 38 is defined to recognize the modifications introduced during the first mutagenesis with oligonucleotide No. 35 and oligonucleotide No. 42 is defined to recognize the modifications introduced during the first two mutageneses with oligonucleotides No. 35 and 38.

Mutant No. 20: The creation of mutant No. 20 requires three successive series of site-directed mutageneses according to the protocol below, using oligonucleotide No. 37 in the first mutagenesis, oligonucleotide No. 38 in the second and oligonucleotide No 43 in the third. Oligonucleotide No. 38 is defined to recognize the modifications introduced during the first mutagenesis with oligonucleotide No. 37 and oligonucleotide No. 43 is defined to recognize the modifications introduced during the first two mutageneses with oligonucleotides No. 37 and 38.

Mutant No. 21: The creation of mutant No. 21 requires three successive series of site-directed mutageneses according to the protocol below, using oligonucleotide No. 37 in the first mutagenesis, oligonucleotide No. 39 in the second and oligonucleotide No 44 in the third. Oligonucleotide No. 39 is defined to recognize the modifications introduced during the first mutagenesis with oligonucleotide No. 37 and oligonucleotide No. 44 is defined to recognize the modifications introduced during the first two mutageneses with oligonucleotides No. 37 and 39.

According to this protocol, the oligonucleotides intended to create mutants No. 17 to No. 21 described in Table 5 are divided up into three categories, 1st series oligonucleotides, 2nd series oligonucleotides and 3rd series oligonucleotides. This division is as follows:

| | |
|---|---|
| 1st series oligonucleotides: | oligonucleotides No. 35, 36 and 37 |
| 2nd series oligonucleotides: | oligonucleotides No. 38, 39, 40 and 41 |
| 3rd series oligonucleotides: | oligonucleotides No. 42, 43 and 44. |

### 3-3-Creation of Pepsin Cleavage Sites in the α6-α7 Inter-helix Loop

The substitution of several amino acids within the α6-α7 inter-helix loop requires, for each of the mutants, only one mutagenesis. The mutagenesis oligonucleotides required to create the examples of mutants given in Table 6 are presented below (numbered from 45 to 52).

| | |
|---|---|
| Oligonucleotide No. 45: | ggt tta gat cgt tta TTa gAa TTa aat act gaa agt tgg |
| | Gly Leu Asp Arg Leu Leu Glu Leu Asn Thr Glu Ser Trp |
| Oligonucleotide No. 46: | ggt tta gat cgt tta TTa TTT TTa aat act gaa agt tgg |
| | Gly Leu Asp Arg Leu Leu Phe Leu Asn Thr Glu Ser Trp |
| Oligonucleotide No. 47: | ggt tta gat cgt tta GAa TTa TTa aat act gaa agt tgg |
| | Gly Leu Asp Arg Leu Glu Leu Leu Asn Thr Glu Ser Trp |
| Oligonucleotide No. 48: | ggt tta gat cgt tta TTa TTT TTT aat act gaa agt tgg |
| | Gly Leu Asp Arg Leu Leu Phe Phe Asn Thr Glu Ser Trp |
| Oligonucleotide No. 49: | ggt tta gat cgt tta GAa GAa TTa aat act gaa agt tgg |
| | Gly Leu Asp Arg Leu Glu Glu Leu Asn Thr Glu Ser Trp |
| Oligonucleotide No. 50: | ggt tta gat cgt tta GAa TTT TTa aat act gaa agt tgg |
| | Gly Leu Asp Arg Leu Glu Phe Leu Asn Thr Glu Ser Trp |
| Oligonucleotide No. 51: | ggt tta gat cgt tta GAa TTT GAa aat act gaa agt tgg |
| | Gly Leu Asp Arg Leu Glu Phe Glu Asn Thr Glu Ser Trp |
| Oligonucleotide No. 52: | ggt tta gat cgt tta GAa gAa GAa aat act gaa agt tgg |
| | Gly Leu Asp Arg Leu Glu Glu Gln Asn Thr Glu Ser Trp |

Oligonucleotide No. 45 is used to create mutant No. 22.
Oligonucleotide No. 46 is used to create mutant No. 23.
Oligonucleotide No. 47 is used to create mutant No. 24.
Oligonucleotide No. 48 is used to create mutant No. 25.
Oligonucleotide No. 49 is used to create mutant No. 26.
Oligonucleotide No. 50 is used to create mutant No. 27.
Oligonucleotide No. 51 is used to create mutant No. 28.
Oligonucleotide No. 52 is used to create mutant No. 29.

The complete protocol for producing these mutants is identical to that described in Example 2. This protocol is common to each of the series of mutageneses, only the mutagenesis oligonucleotide and the oligonucleotide for inhibition/restoration of the resistance to the antibiotic change.

### Example 4:

### Creation of Pepsin Cleavage Sites in the α3-α4, α4-α5, α5-α6 and α6-α7 Inter-helix Loops of Various Cry Toxins

Several groups of Cry proteins exhibit structural similarities. They are in particular the proteins belonging to the Cry1, Cry3, Cry4, Cry7, Cry8, Cry9, Cry10, Cry16, Cry17, Cry19 or Cry20 families. These similarities are demonstrated in the literature (Schnepf et al., 1998). Other Cry proteins not cited in the literature can also exhibit structural and sequence similarities with these families. The aim of Example 4 is to demonstrate the applicability of the teaching of the present invention, as exemplified on the Cry9Ca1 protein in Examples 2 and 3, to all these structurally similar families.

The modifications in the inter-helix loops described in Examples 2 and 3 can be carried out in an equivalent manner for all the Cry proteins in which it is possible to identify inter-helix loops similar to those present in domain I of the Cry9Ca1 toxin. If the location and the sequence of these inter-helix loops are defined for these various Cry toxins, it is very easy for those skilled in the art to form modifications similar to those given in Examples 2 and 3 using the technical details provided in these same Examples 2 and 3. In the present example, the elements for creating specific sites for degradation by pepsin in the Cry toxins other than the Cry9Ca1 toxin, and in particular the Cry1, Cry3, Cry4, Cry7, Cry8, Cry9, Cry10, Cry16, Cry17, Cry19 and Cry20 proteins, are given. The modification of these inter-helix loops to create sites for degradation by pepsin in the Cry1, Cry3, Cry4, Cry7, Cry8, Cry9, Cry10, Cry16, Cry17, Cry19 or Cry20 toxins requires the following steps to be followed:

1) Establish, according to the sequences and the locations of the inter-helix loops given in Tables 6-13 below, lists of possible mutants having one or more leucine, phenylalanine or glutamic acid residues as given in Tables 1, 4, 5 and 6 and in Examples 2 and 3.

2) Establish the sequences of the mutant genes taking into account the codon preference of the host organism and, if this organism is B. thuringiensis, preferentially using the codons TTA, TTT and GAA for leucine, phenylalanine and glutamic acid, respectively.

3) Synthesizing mutagenesis oligonucleotides for modifying the sequence of the genes encoding the toxins selected based on the model for those given in Examples 2 and 3.

4) Use single or multiple mutagenesis strategies as described in Examples 2 and 3 and according to the experimental protocols described in detail in Examples 2 and 3.

The location of the α3-α4, α4-α5, α5-α6 and α6-α7 inter-helix loops of domain I and their sequences are given, for the Cry1, Cry3, Cry4, Cry7, Cry8, Cry9, Cry10, Cry16, Cry17, Cry19 and Cry20 toxins, in Tables 7, 8, 9, 10, 11, 12 and 13 below. These sequences are given for each of the holotype proteins as defined by the Bacillus thuringiensis classification committee (Crickmore et al., 2001). However, since the intra-holotype sequence homologies, i.e. the sequence homologies between the various subtypes of the same holotype, are very high, those skilled in the art will be able to adapt the teaching of the present Example 4 to all the Cry protein subtypes.

TABLE 7
Location and sequence of the α3-α4
inter-helix loop in the Cry1 proteins

| | Amino acid | Position in | Nucleotide | |
|---|---|---|---|---|
| Protein | sequence | protein | sequence | Position in gene |
| CrylAa | DPTN | 120 to 123 | gatcctactaat | 358 to 369 |
| CrylAb | DPTN | 120 to 123 | gatcctactaat | 358 to 369 |
| CrylAc | DPTN | 120 to 123 | gatcctactaat | 358 to 369 |
| CrylAd | DPTN | 120 to 123 | gatcctactaat | 358 to 369 |
| CrylAe | DPTN | 120 to 123 | gatcctactaat | 358 to 369 |
| CrylAf | DPTN | 120 to 123 | gatcctactaat | 358 to 369 |
| CrylAg | DPTN | 120 to 123 | gatcctactaat | 358 to 369 |
| CrylBa | NRDD | 139 to 142 | aaccgtgatgat | 415 to 426 |
| CrylBb | NRND | 144 to 147 | aaccgaaatgat | 430 to 441 |
| CrylBc | NRND | 144 to 147 | aaccgaaatgat | 430 to 441 |
| CrylBd | NRND | 144 to 147 | aaccgaaatgat | 430 to 441 |
| CrylCa | DPNN | 119 to 122 | gatcctaataat | 355 to 366 |
| CrylCb | DPDN | 119 to 122 | gatcctgataat | 355 to 366 |
| CrylDa | DPTN | 119 to 122 | gatcctactaat | 355 to 366 |
| CrylDb | DPSN | 119 to 122 | gatccgtctaat | 355 to 366 |
| CrylEa | DPTN | 118 to 121 | gatcctactaat | 352 to 363 |
| CrylEb | DPTN | 117 to 120 | gatcctactaat | 349 to 360 |
| CrylFa | NPNN | 118 to 121 | aatcctaataat | 352 to 363 |
| CrylFb | NPNN | 118 to 121 | aatcctaataat | 352 to 363 |
| CrylGa | DPNN | 118 to 121 | gatcctaataat | 352 to 363 |
| CrylGb | DPDN | 118 to 121 | gatcctgataac | 352 to 363 |
| CrylHa | SPNN | 122 to 125 | tctcctaataat | 364 to 375 |
| CrylHb | SPNN | 121 to 124 | tctcctaataat | 361 to 372 |
| CrylIa | NRNN | 148 to 151 | aatcgtaataac | 442 to 453 |
| CrylIb | NRNN | 148 to 151 | aatcgtaataac | 442 to 453 |
| CrylIc | NRNN | 148 to 151 | aatcgtaataac | 442 to 453 |
| CrylId | NRNN | 148 to 151 | aatcgcaataac | 442 to 453 |
| CrylIe | NRNN | 148 to 151 | aatcgcaacaac | 442 to 453 |
| CrylJa | DPDN | 119 to 122 | gatcctgataac | 355 to 366 |
| CrylJb | TPDN | 119 to 122 | actccagataac | 355 to 366 |
| CrylKa | NRND | 145 to 148 | aaccgaaatgat | 433 to 444 |

TABLE 8
Location and sequence of the α4-α5 inter-helix loop in the Cry1 proteins

| | Amino acid | Position in | | |
|---|---|---|---|---|
| Protein | sequence | protein | Nucleotide sequence | Position in gene |
| Cry1Aa | LAVQNYQVPLL | 148 to 158 | ttggcagttcaaaattatcaagttcctctttta | 442 to 474 |
| | FLAVQNYQVPLL | 148 to 158 | ttgcagttcaaaattatcaagttcctctttta | 442 to 474 |
| Cry1Ab | FAVQNYQVPLL | 148 to 158 | ttgcagttcaaaattatcaagttcctctttta | 442 to 474 |
| Cry1Ac | FAVQNYQVPLL | 148 to 158 | tttgcagttcaaaattatcaagttcctctttta | 442 to 474 |
| | LAVQNYQVPLL | 148 to 158 | ttggcagtcaaaattatcaagttcctctttta | 442 to 474 |
| Cry1Ad | FTVQNYQVPLL | 148 to 158 | cctacagttcaaaattatcaagtacctcttcta | 442 to 474 |
| Cry1Ae | FTVQNYQVPLL | 148 to 158 | tttacagttcaaaattatcaagtacctcttcta | 442 to 474 |
| Cry1Af | FAVQNYQVPLL | 148 to 158 | tttgcagttcaaaattatcaagttcctctttta | 442 to 474 |
| Cry1Ag | LAVQNYQVPLL | 148 to 158 | ttggcagttcaaaattatcaagttcctctttta | 442 to 474 |
| Cry1Ba | FAIRNQEVPLL | 167 to 177 | ttcgcaattagaaaccaagaagttccattattg | 499 to 531 |
| Cry1Bb | FRIRNEEVPLL | 172 to 182 | ttcagaatacgaaatgaagaagttccattatta | 514 to 546 |
| Cry1Bc | FRIRNEEVPLL | 172 to 182 | ttcagaatacgaaatgaagaagttccattatta | 514 to 546 |
| Cry1Bd | FRIRNEEVPLL | 172 to 182 | ttcagaatacgaaatgaagaagttccattatta | 514 to 546 |
| Cry1Ca | FRISGFEVPLL | 147 to 157 | tttcgaatttctggatttgaagtacccctttta | 439 to 471 |
| Cry1Cb | FRIAGFEVPLL | 147 to 157 | tttcgaattgctggatttgaagtacccctttta | 439 to 471 |
| Cry1Da | FRVQNYEVALL | 147 to 157 | tttagagttcaaaattatgaagttgctctttta | 439 to 471 |
| Cry1Db | LRVRNYEVALL | 147 to 157 | ttaagagttcgtaattatgaagttgctctttta | 439 to 471 |
| Cry1Ea | LFSVQNYQVPFL | 145 to 156 | aattttacacttacaagttttgaaatccctctttta | 433 to 468 |
| Cry1Fb | NFTLTSFEIPLL | 145 to 156 | aattttacacttacaagttttgaaatccctctttta | 433 to 468 |
| Cry1Ga | RLAIRNLEVVNL | 145 to 156 | actttggcaattcggaatcttgaggtagtgaattta | 433 to 468 |
| Cry1Gb | LMAIPGPELATL | 145 to 156 | cttatggcaattccaggtttgaattagctactttta | 433 to 468 |
| Cry1Gb | LMAIPGPELATL | 145 to 156 | cttatggcaattccaggttttgaattagctacttta | 433 to 468 |
| Cry1Ha | LREQGFEIPLL | 150 to 160 | ctgagagaacaaggctttgaaattcctctttta | 448 to 480 |
| Cry1Hb | LREQGFEIPLL | 149 to 159 | ctgagagaacagggctttgaaattcctctttta | 445 to 477 |
| Cry1Ia | FAVSGEEVPLL | 176 to 186 | tttgcagtgtctggagaggaggtaccattatta | 526 to 558 |
| Cry1Ib | FAVSGEEVPLL | 176 to 186 | tttgcagtatctggtgaggaagtaccattatta | 526 to 558 |
| Cry1Ic | FAVSGEEVPLL | 176 to 186 | tttgcagtatctggtgaggaagtaccattatta | 526 to 558 |
| Cry1Id | FAVSGEEVPLL | 176 to 186 | tttgcagtttctggagaagaggtgccgctatta | 526 to 558 |
| Cry1Ie | FAVSGEEVPLL | 176 to 186 | tttgcagtatcaggtgaggaagtaccattattg | 526 to 558 |
| Cry1Ja | FRIIGFEVPLL | 147 to 157 | tttcggataattggatttgaagtgccactttta | 439 to 471 |
| Cry1Jb | FRIPGFEVPLL | 147 to 157 | tttcggattcccggatttgaagtgccacttcta | 439 to 471 |
| Cry1Ka | FSIRNEEVPLL | 173 to 183 | ttcagcatacgaacgaagaggttccattattta | 517 to 549 |

TABLE 9
Location and sequence of the α5-α6 inter-helix loop in the Cry1 proteins

| | Amino acid | Position in | | |
|---|---|---|---|---|
| Protein | sequence | protein | Nucleotide sequence | Position in gene |
| Cry1Aa | FGQRWGFD | 178 to 185 | tttggacaaaaggtggggatttgat | 532 to 555 |
| Cry1Ab | FGQRWGFD | 178 to 185 | tttggacaaaggtggggatttgat | 532 to 555 |
| Cry1Ac | FOQRWGPD | 178 to 185 | tttggacaaaggtggggatttgat | 532 to 555 |
| Cry1Ad | FGQRWGFD | 178 to 185 | ttggacaacgttggggatttgat | 532 to 555 |
| Cry1Ae | FGQRWGLD | 178 to 185 | tttggacaacgttggggacttgat | 532 to 555 |
| Cry1Af | CGQRSGFD | 175 to 182 | tgtggacaaaggtcgggatttgat | 523 to 546 |
| Czy1Ag | FGQRWGFD | 178 to 185 | tttggacaaaggtggggatttgat | 532 to 555 |
| Cry1Ba | FGSEFGLT | 197 to 204 | ttggtagtgaatttgggcttaca | 589 to 612 |
| Cry1Bb | FGSEWGMA | 202 to 209 | tttggtagtgaatgggggatggca | 604 to 627 |
| Cey1Bc | FGSEWGMA | 202 to 209 | tttggtagtgaatgggggatggca | 604 to 627 |
| Cry1Bd | FGSEWGMA | 202 to 209 | tttggtagtgaatgggggatggca | 604 to 627 |
| Cry1Ca | FGERWGLT | 177 to 184 | ttggagaaagatggggattgaca | 529 to 552 |
| | FGERWGVT | 177 to 184 | ttggagaaagatggggagtgaca | 529 to 552 |
| Cry1Cb | FGARWGLT | 177 to 184 | tttggagcaagatggggattgaca | 529 to 552 |
| Cry1Da | FGERWGYD | 177 to 184 | ttcggagaaagatggggatatgat | 529 to 552 |
| Cry1Db | YGQRWGFD | 177 to 184 | tacggtcagagatggggctttgac | 529 to 552 |
| Cry1Ea | FGQAWGFD | 176 to 183 | tttgggcaggcttggggatttgat | 526 to 549 |
| Cry1Eb | FGQRWGFD | 175 to 182 | tttggacaacgttggggatttgat | 523 to 546 |
| Cry1Fa | FGQGWGLD | 176 to 183 | tttgggcagggttggggactggat | 526 to 549 |
| Cry1Fb | FGQGWGLD | 176 to 183 | tttgggcagggttggggctggat | 526 to 549 |
| Cry1Ga | FGERWGLT | 176 to 183 | tttggagaaagatggggattaaca | 526 to 549 |
| Cry1Gb | FGERWGLT | 176 to 183 | tttggggagagatggggattgaca | 526 to 549 |
| Cry1Ha | FGQRWGLD | 180 to 187 | tttgggcaaagatggggacttgac | 538 to 561 |
| Cry1Hb | FGQRWGLD | 179 to 186 | tttggacagagatggggacttgat | 535 to 558 |
| Cry1Ia | FGKEWGLS | 206 to 213 | tttggaaaagagtggggattatca | 616 to 639 |
| Cry1Ib | FGKEWGLS | 206 to 213 | tttgaaagaatggggattatca | 616 to 639 |
| Cry1Ic | FEKNGGLS | 206 to 213 | ttgaaaagaatgggggattatca | 616 to 639 |
| Cry1Id | FGKEWGLS | 206 to 213 | tttggaaaagaatgggggattgtca | 616 to 639 |
| Cry1Ie | FGKEWGLS | 206 to 213 | tttggaaaagagtggggattatct | 616 to 639 |
| Cry1Ja | FGERWGLT | 177 to 184 | ttggagagagatggggattgacg | 529 to 552 |
| Cry1Jb | FGERWGLT | 177 to 184 | ttcggagagagatggggattgacg | 529 to 552 |
| Cry1Ka | FGSEWGMS | 203 to 210 | tttggtagtgaatgggggatgtca | 607 to 630 |

TABLE 10
Location and sequence of α6-α7 inter-helix loop
in the Cry1 proteins

| | Amino acid | Position in | | |
|---|---|---|---|---|
| Protein | sequence | protein | Nucleotide sequence | Position in gene |
| Cry1Aa | VWGPD | 218 to 222 | gtatggggaccggat | 652 to 666 |
| Cry1Ab | VWGPD | 218 to 222 | gtatggggaccggat | 652 to 666 |
| Cry1Ac | VWGPD | 218 to 222 | gtatggggaccggat | 652 to 666 |
| Cry1Ad | VWGPD | 218 to 222 | gtatggggaccggaa | 652 to 666 |
| Cry1Ae | VWGPD | 218 to 222 | gtatggggaccggat | 652 to 666 |
| Cry1Af | VWGPD | 215 to 219 | gtatggggaccggat | 643 to 657 |
| Cry1Ag | VWGPD | 218 to 222 | gtatggggacecgac | 652 to 666 |
| Cry1Ba | LRGTN | 237 to 241 | ttgagagggacaaaa | 709 to 723 |
| Cry1Bb | LRGTN | 242 to 246 | ttaagagggacaaat | 724 to 738 |
| Cry1Bc | LRGTN | 242 to 246 | ttaagagggacaaat | 724 to 738 |
| CrylBd | LRGTN | 242 to 246 | ttaagagggacaaat | 724 to 738 |
| Cry1Ca | LPKST | 217 to 221 | ttaccgaaatctacg | 649 to 663 |
| Cry1Cb | LPKST | 217 to 221 | ttaccaaaatcracg | 649 to 663 |
| Cry1Da | LEGRF | 217 to 221 | ttaggaaggtcgtttt | 649 to 663 |
| Cry1Db | LEGSR | 217 to 221 | ttagagggatctcga | 649 to 663 |
| Cry1Ea | LPRTGG | 216 to 221 | ttaccacgaactggtggg | 646 to 663 |
| Cry1Eb | LPRNEG | 215 to 220 | ttaccacgtaatgaaggg | 643 to 660 |
| CrylFa | LRGTNT | 216 to 221 | ataagaggtaataatact | 646 to 663 |
| Cry1Fb | LRGTNT | 216 to 221 | ttaagaggtactaatact | 646 to 663 |
| Cry1Ga | IGGIS | 216 to 220 | attggagggataagt | 646 to 660 |
| Cry1Gb | LNVIR | 216 to 220 | ttaaatgttataaga | 646 to 660 |
| Cry1Ha | FGGVS | 220 to 224 | tttggtggtgtgtca | 658 to 672 |
| Cry1Hb | FGVVT | 219 to 223 | tttggtgttgtaaca | 655 to 669 |
| Cry1Ia | LRGTN | 246 to 250 | ttgaggggtacaaat | 736 to 750 |
| Cry1Ib | LRGTN | 246 to 250 | ttgaggggtacaaat | 736 to 750 |
| Cry1Ic | LRATN | 246 to 250 | ttgagggctacaaat | 736 to 750 |
| Cry1Id | LRGTN | 246 to 250 | ttgaggggaacaaat | 736 to 750 |
| Cry1Ie | LRGTN | 246 to 250 | ttgagaggtacaaat | 736 to 750 |
| Cry1Ja | LGPRS | 217 to 221 | cagggtttagatct | 649 to 663 |
| Cry1Jb | LGFTS | 217 to 221 | ctagggtttacttct | 649 to 663 |
| Cry1Ka | LRGTT | 243 to 247 | ttaagagggacaact | 727 to 741 |

TABLE 11
Location and sequence of the α3-α4 inter-helix loop
in the Cry3, Cry4, Cry7, Cry8, Cry9, Cry10, Cry16, Cry17, Cry19
and Cry20 proteins

| | Amino acid | Position in | | |
|---|---|---|---|---|
| Protein | sequence | protein | Nucleotide sequence | Position in gene |
| Cry3Aa | NPVSSRN | 153 to 159 | aatcctgtgagulcacgaaat | 457 to 477 |
| Cry3Ba | APVNLRS | 154 to 160 | gcgcctgtaaatttacgaagt | 460 to 480 |
| Cry3Eb | TPLSLRS | 154 to 160 | acacctttaagtttgcgaagt | 440 to 480 |
| Cry3Ca | TPLTLRD | 151 to 157 | actcctttgactttcgagat | 451 to 471 |
| Cry4Aa | NNPNPQNTQD | 160 to 169 | aaataatcaaacccacaaaatactcaggat | 478 to 501 |
| Cry4Ba | EPNNQSYRTA | 136 to 145 | gagcctaataaaccagtcctatagaacagca | 406 to 435 |
| Cry7Aa | KQDDPEAILS | 147 to 156 | aaacaagatgatccagaagctatactttct | 439 to 468 |
| Cry7Ab | NPDDPATITR | 147 to 156 | aatcctgatgaccagcaactataacacga | 439 to 468 |
| Cry8Aa | NRNDARTRSV | 158 to 167 | aatcgcaatgatgcaagaactaagtgtt | 472 to 501 |
| Czy8Ba | NPNGSRALRD | 159 to 168 | aatccaaatggttcaagagccttacgagat | 415 to 504 |
| Cry8Ca | NPHSTRSAAL | 159 to 168 | aacccacacagtacacgaagcgcagcactt | 475 to 504 |
| Cry9Aa | NPNSASAEEL | 146 to 155 | aatcctaattctgcttctgctgaagaactc | 436 to 465 |
| Cry9Ba | RPNGVRANLV | 134 to 143 | agaccaacggcgtaagagcaaacttagtt | 400 to 429 |
| Cry9Ca | DRNDTRNLSV | 159 to 168 | gatcgaaacgatacacgaaatttaagtgtt | 475 to 504 |
| Cry9Da | RPNGARASLV | 159 to 168 | agaccaaatggcgcaagggcatccttagtt | 475 to 504 |
| Cry9Ea | RPNGARANLV | 159 to 168 | agaccgaacggagcaagagctaacttagtt | 475 to 504 |
| Cry10Aa | ARTHANAKAV | 162 to 171 | gcacgtacacacgctaatgctaaagcagta | 484 to 513 |
| Cry16Aa | NYNPTSIDDV | 109 to 118 | aattataatccaacttctatagacgatgta | 325 to 354 |
| Cry17Aa | NKDDPLAIAEL | 127 to 131 | aataaagatgaccccttggctatagctgaatta | 379 to 411 |
| Cry19Aa | DPKSTGNLSTL | 159 to 169 | gatccaaaatacaggtaatttaagcacctta | 475 to 507 |
| Cry19Ba | NKNNFASGEL | 151 to 160 | aataaaaataatttcgcaagtggtgaactt | 451 to 480 |
| Cry20Aa | ERNRTRENGQ | 141 to 150 | gaacgtaatagaactcgtgaaaacggacaa | 421 to 450 |

TABLE 12
Location and sequence of the α4-α5 inter-helix loop
in the Cry3, Cry4, Cry7, Cry8, Cry9, Cry10, Cry16, Cry17, Cry19 and Cry20 proteins

| | Amino acid | Position | | Position |
|---|---|---|---|---|
| Protein | sequence | in protein | Nucleotide sequence | in gene |
| Cry3Aa | ISGYEVL | 186 to 192 | atttctggatacgaggttcta | 556 to 576 |
| CRy3Ba | VSKFEVL | 157 to 193 | gtttccaaattcgaagttctg | 559 to 579 |
| CRY3Bb | VSKFEVL | 187 to 193 | gtttccaaattcgaagtgctg | 559 to 579 |
| Cry3Ca | VSGYEVL | 184 to 190 | gtctctggatacgaagttcta | 550 to 570 |
| Cry4Aa | LVNSCPPNPSDCDYYNILVL | 188 to 207 | cttgtaaactcttgtcctcctaatcctagtgattgcgattactataacat | 562 to 621 |
| | | | actagtatta | |
| Cry4Ba | FSNLVGYELLLL | 164 to 175 | tttagcaacttagtaggttatgaattattgttatta | 490 to 525 |
| Cry7Aa | FKVTGYEIPLL | 175 to 185 | tttaaggttactggatatgaaataccattacta | 523 to 555 |
| Cry7Ab | FRVAGYEIPLL | 175 to 185 | tttagggttgctggatatgaaataccattacta | 523 to 555 |
| Cry8Aa | FAVSGHEVLLL | 136 to 196 | tttgcagtatccggacacgaagtactattatta | 556 to 588 |
| Cry8Ba | FRVTNFEVPFL | 187 to 197 | tttcgagtgacaaauttgaagtaccatttcctt | 559 to 591 |
| Cry8Ca | FSQTNYETPLL | 187 to 197 | ttttacaaacgaattatgagactccactctta | 559 to 591 |
| Cry9Aa | LTNGGSLARQNAQILLL | 175 to 191 | ttaacgaatggtggtctgttagctagacaaaatgcccaaatattattatt | 523 to 571 |
| | | | a | |
| Cry9Ba | FGSGPGSQRFQAQLL | 161 to 175 | tttggtagtggccctggaagtcaaaggtttcaggcacaatgttg | 481 to 525 |
| Cry9Ca | FAVNGQQVPLL | 187 to 197 | tttgcagtaaatggcacagcaggttccattactg | 559 to 591 |
| Cry9Da | FGSGPGSQRJYATILL | 116 to 200 | tttggctctggtcctggaagtcaaaattatgcaactatattactt | 556 to 600 |
| Cry9Ea | FGTOPOSQIWAVALL | 186 to 200 | tttggtacgggtcctggtagtcaaagagatgcggtagcgttgttg | 556 to 600 |
| Cry10Aa | LKNNASYRIPTL | 189 to 200 | ttaaaaaataatgctagctatcgfaataccaacactc | 565 to 600 |
| Cry16Aa | FKVKNYEVTVL | 136 to 146 | tttaaggttaaaaattatgaagtaacagtgtta | 406 to 438 |
| Cry17Aa | FKRANYEVLLL | 155 to 165 | tttaaaagggcgaattatgaagtcttactatta | 463 to 495 |
| Cry19Aa | VNNQOSPOYELLLL | 187 to 200 | gttaaataatcaggggagtccaggttatgagttacttttattg | 559 to 600 |
| Cry19Ba | FSLGGYETVLL | 180 to 190 | ttctcattaggaggttatgaaacagtattatta | 538 to 570 |
| Cry20Aa | LSRRGFETLLL | 173 to 183 | ctttctcgcagaggattcgaaactcttttatta | 517 to 549 |

TABLE 13
Location and sequence of the α5-α6 inter-helix loop in the Cry3, Cry4, Cry7,
Cry8, Cry9, Cry10, Cry16, Cry17, Cry19 and Cry20 proteins

| | Amino acid | Position in | | |
|---|---|---|---|---|
| Protein | sequence | protein | Nucleotide sequence | Position in gene |
| Cry3Aa | GEEWGYE | 215 to 222 | ggagaagantggggatacgaa | 643 to 663 |
| Cry3Ba | GEEWGYS | 216 to 222 | ggagaagaatggggatattct | 646 to 666 |
| Cry3Bb | GEEWGYS | 226 to 222 | ggagaagaatggggatattct | 646 to 666 |
| Cry3Ca | GTDWGYS | 213 to 219 | ggaacggattggggatattct | 637 to 657 |
| Cry4Aa | FEAYLKNNRQFDYEL | 227 to 241 | tttgaagcgtatttaaaaaacaattcgattattttagag | 679 to 723 |
| Cry4Ba | LINAQEWSL | 193 to 201 | ctcataaatgcacaagaatggtcttta | 577 to 603 |
| | PHKCTRMVY | 193 201 | cctcataaatgcacaagaatggtctat | 577 to 603 |
| Cry7Aa | GDKWGF | 206 to 211 | ggagataaatggggattc | 616 to 633 |
| | GDKWEF | 206 to 221 | ggagataaatgggaattc | 616 to 633 |
| Cry7Ab | GDKWGF | 206 to 211 | ggagataaatgggguttc | 616 to 633 |
| Cry8Aa | GEEWGF | 217 to 222 | ggagaagagtggggattt | 649 to 666 |
| Cry8Ba | GEEWGL | 218 to 223 | ggagaagaatggggattg | 652 to 669 |
| Cry8Ca | GKEWGY | 218 to 223 | gggaaggaatggggatat | 652 to 669 |
| Cry9Aa | RYGTNWGL | 210 to 217 | agatatggcactaattgggggcta | 628 to 651 |
| Cry9Ba1 | KYGARWGL | 194 to 201 | aagtatggggcaagatggggatc | 580 to 603 |
| Cry9Ca | LFGEGWGF | 216 to 223 | ctttttggagaaggatggggattc | 646 to 669 |
| Cry9Da | IYGARWGL | 219 to 226 | atttatggagcagatgggggctg | 655 to 678 |
| Cry9Ea | IYGARWOL | 219 to 226 | atctaggggcaagatggggactt | 655 to 678 |
| Cry10Aa | TYYNIWLQ | 219 to 226 | acttatggggcaagatggggactt | 655 to 673 |
| Cry16Aa | IYGDAWNLYRELGP | 265 to 178 | atttatggagatgcatggaatttatatagagaattaggattt | 493 to 534 |
| Cry17Aa | LLNKVIDNF | 184 to 192 | cttttaaataagttatagataatttt | 550 to 576 |
| Cry19Aa | IYGDKWWSA | 219 to 227 | atttatggagataaatggtggagcgca | 655 to 681 |
| Cry19Ba | IYGKELG | 209 to 215 | atttacggaaaagaattagga | 625 to 645 |
| Cry20Aa | LYRNQWL | 202 to 208 | ctttatagaaatcaatggtta | 604 to 624 |

TABLE 14
Location and sequence of the α6-α7 inter-helix loop in
the Cry3, Cry4, Cry7, Cry8, Cry9, Cry10, Cry16, Cry17, Cry19
and Cry20 proteins

| | Amino acid | Position in | | |
|---|---|---|---|---|
| Protein | sequence | protein | Nucleotide sequence | Position in gene |
| Cry3Aa | RGSS | 255 to 258 | agaggttcatct | 763 to 774 |
| Cry3Ba | RGST | 256 to 259 | agaggttcaact | 766 to 777 |
| Cry3Bb | RGST | 256 to 259 | agaggttcaat | 766 to 777 |
| Cry3Ca | RGST | 253 to 256 | agaggttcgact | 757 to 768 |
| Cry4Aa | LIKTTPD | 274 to 280 | ttaattaaaacgacgcctgat | 820 to 840 |
| Cry4Ba | LRNKS | 235 to 239 | cttagaaataaatct | 703 to 717 |
| Cry7Aa | LNGST | 245 to 249 | ttgaacggttccact | 733 to 747 |
| Cry7Ab | LNGST | 245 to 249 | ttgaacggttccact | 733 to 747 |
| Cry8Aa | LKGTT | 256 to 260 | ttgaaggtaccact | 766 to 780 |
| Cry8Ba | LKGSS | 257 to 261 | ttaaaaggctcgagc | 769 to 783 |
| Cry8Ca | LRGTG | 257 to 261 | ttaagaggaacgggt | 769 to 783 |
| Cry9Aa | LRQRGTS | 252 to 258 | ctaagacaacgaggcactagt | 754 to 774 |
| Cry9Ba1 | LRGTS | 236 to 240 | ttacgaggaacgagc | 706 to 720 |
| Cry9Ca | LRGTN | 257 to 261 | ttaagaggaacaaat | 769 to 783 |
| Cry9Da | LRGTT | 260 to 264 | ttaagaggcacaacc | 778 to 792 |
| Cry9Ea | VRGTN | 260 to 264 | gtagaggaacaaat | 778 to 792 |
| Cry10Aa | 1RTNT | 267 to 271 | attagaactaatact | 799 to 813 |
| Cry16Aa | LKLDPN | 210 to 215 | ttaaaactagatccgaat | 628 to 645 |
| Cry17Aa | IKNKTRDF | 224 to 231 | ataaaaaataaaactagggatttt | 670 to 693 |
| Cry19Aa | FRTAG | 261 to 265 | ttagaacagcaggt | 781 to 795 |
| Cry19Ba | KKQIG | 250 to 254 | aaaaaacaaatagga | 748 to 162 |
| Cry20Aa | DRSS | 245 to 248 | gatcgttcaagt | 733 to 744 |

Mutants can be prepared for each of the cry genes mentioned in this example, based on the models of Examples 1, 2 and 3. The technical procedures which can be used to carry out the mutagenesis are similar to those given in Examples 1, 2 and 3.

### Example 5

### Overall Increase in the Leucine, Phenylalanine and Glutamic Acid Content of the Cry Proteins

The overall increase in the leucine, phenylalanine and glutamic acid content of the Cry proteins is described below for the Cry9Ca1 toxin. Although this example is carried out on the Cry9Ca1 protein and the cry9Ca1 gene, its teaching is applicable to all the Cry toxins and all the cry genes. This teaching applies in particular to all the Cry toxins the sequence of which is known and filed in the Genbank database:

www.ncbi.nlm.bih.gov/Genbank/index.html.

The Genbank accession numbers for the cry genes are available on the following site:

www.biols.susx.ac.uk/Home/Neil_Crickmore/Bt/index.html.

This teaching also applies to all the Cry toxins and cry genes, the sequences of which are not disclosed on Genbank.

Unlike the strategies described in Examples 1 to 4, the aim is not to modify a precise region of the toxin so as to integrate amino acids recognized by pepsin, but to increase, overall, the number of these sites by increasing the amount of leucine, of phenylalanine and of glutamic acid in said toxin. This strategy makes it possible to make the Cry toxin more sensitive to pepsin by increasing the percentage of residues recognized by pepsin. Glutamic acid (E; Glu) preferentially substitutes for aspartic acid (D; Asp), phenylalanine (F; Phe) preferentially replaces tryptophan (W; Trp) and leucine (L; Leu) preferably replaces valine (V; Val) or isoleucine (I; Ile). This strategy require the creation of a three-dimensional model for the activated Cry9Ca1 toxin, created from the primary sequence of the protein by comparison with the three-dimensional structures of Cry1Aa1 and Cry3Aa1. The model was created using the Swiss-Model Protein Modelling Server (Peitsch, 1995; Peitsch, 1996; Guex and Peitsch, 1997). The server address is as follows:

www.expasy.ch/swissmod/swiss-model.html.

Preferably, the substitutions should reach a maximum level of 25%. The activated Cry9Ca1 toxin contains 31 aspartic acids, 9 tryptophans and 47 valines. There are naturally 26 glutamic acids, 35 phenylalanines and 62 leucines. Taking into account a maximum substitution of 25% for each of the amino acids, the relative ratios are as follows:

| | Number of residues in native | Number of residues in |
|---|---|---|
| Amino acid | Cry9Ca1 | modified Cry9Ca1 |
| Asp (D) | 31 | 24 |
| Glu (E) | 26 | 33 |
| Trp (W) | 9 | 7 |
| Phe (F) | 35 | 37 |
| Val (V) | 47 | 36 |
| Leu (L) | 61 | 72 |

The substitution of isoleucine (I; Ile) with leucine can also be envisioned instead of or in addition to the substitution of valine with leucine. There are naturally 27 isoleucines in the Cry9Ca1 toxin. Taking into account a preferential degree of substitution of 25%, it is sufficient to replace 6 isoleucine residues with leucines.

It is possible to modify the sequence of the cry9Ca1 gene as shown below. The only aim of the demonstration below is to illustrate the example, and it does not in any way limit the scope of the invention. This demonstration relates to aspartic acid, tryptophan and valine residue replacement. Those skilled in the art can very easily adapt this approach to any other cry gene, the sequence of which would be known, and in particular from the sequences available on Genbank and the accession numbers of which are mentioned on the following site:

www.biols.susx.ac.uk/Home/Neil_Crickmore/Bt/index.html.

The cry genes generally expressed in transgenic plants are truncated genes, i.e. only the gene sequence encoding the activated toxin is introduced into these plants. The sequences given in this example correspond to this truncated version and extend, depending on whether it is a case of the gene or the protein, from the initiation codon or from the first methionine to 15 codons or amino acids downstream of the conserved block 5 which limits the activated toxin.

The sequence of the native and truncated cry9Ca1 gene is given in SEQ ID NO:1.

The sequence of the native and truncated Cry9Ca1 protein is given in SEQ ID NO:2.

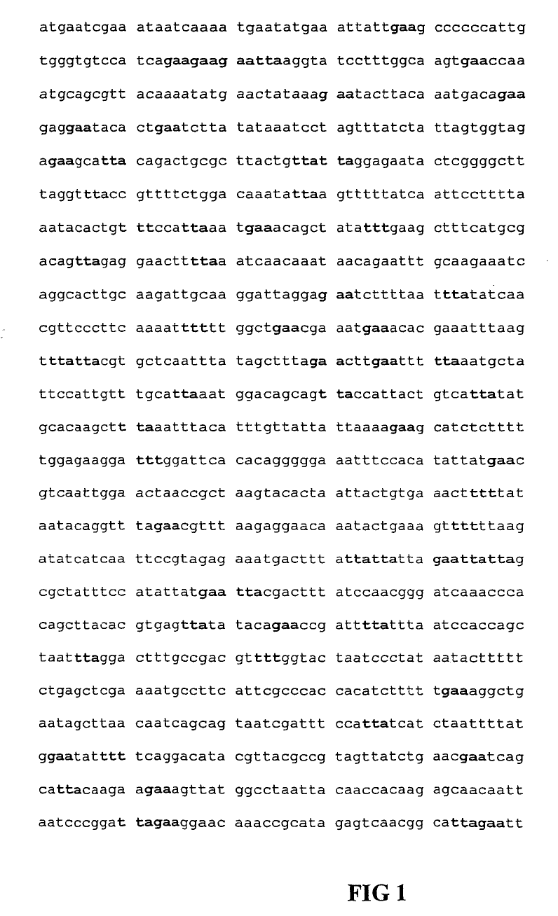

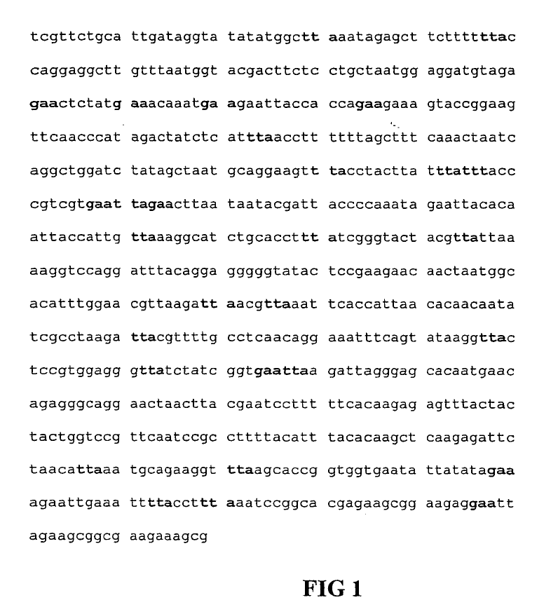

The sequence of a modified cry9Ca1 gene in which all the codons encoding the valine, aspartic acid and tryptophan residues have been modified is given in FIG. 1 (SEQ ID NO:9). This modified sequence can be used as a basis for defining the various mutagenesis oligonucleotides which may be used. The modified bases are represented in bold characters.

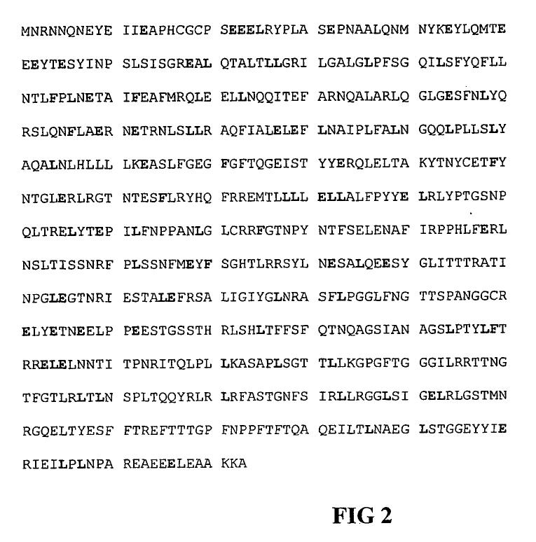

The sequence of a modified Cry9Ca1 protein in which all the valine, aspartic acid and tryptophan residues have been modified is given in FIG. 2 (SEQ ID NO:10) and the modified amino acids are represented in bold characters.

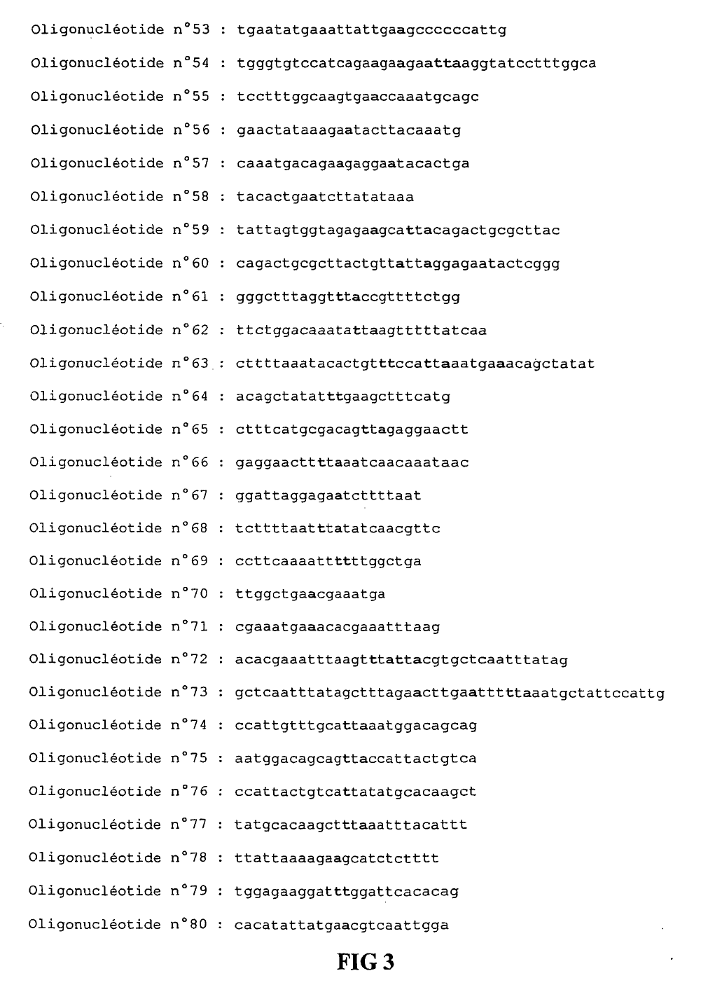

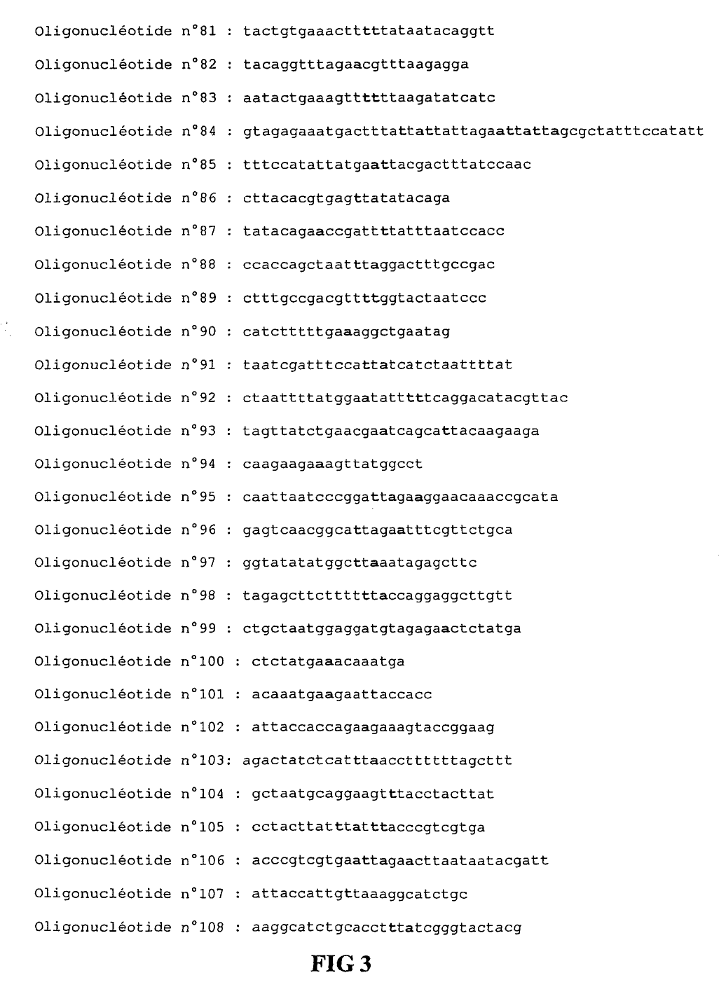

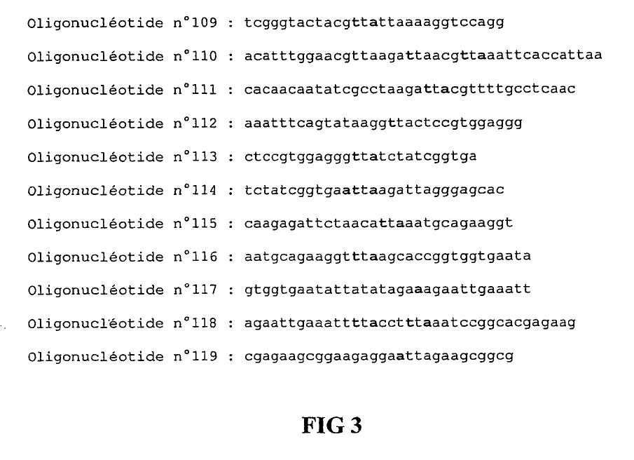

All the mutagenesis oligonucleotides which may make it possible to perform the valine, phenylalanine and glutamic acid residue replacements are given in FIG. 3 (SEQ ID NOS:94 to 160). The modified bases are represented in bold characters.

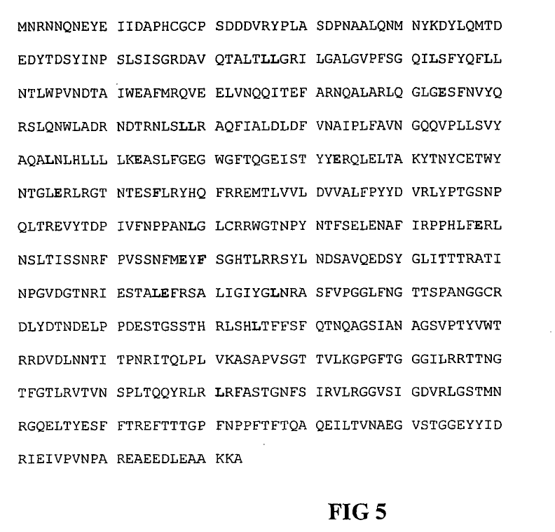

A possibility for the use of certain oligonucleotides to create a modified cry9Ca1 gene in which the replacement with respect to codons encoding the valine, aspartic acid and tryptophan residues has been carried out at up to 25% is shown below by way of illustration. The aim of this illustration is to exemplify the strategy developed without limiting the scope of the invention. On the basis of the teaching of this example and of FIGS. 1 to 3 (SEQ ID NOS:9 and 10), those skilled in the art will be able to adapt other combinations of the oligonucleotides given in FIG. 5 (SEQ ID NOS:94 to 160) or other oligonucleotides prepared along the same principle, in particular for replacing isoleucine residues.

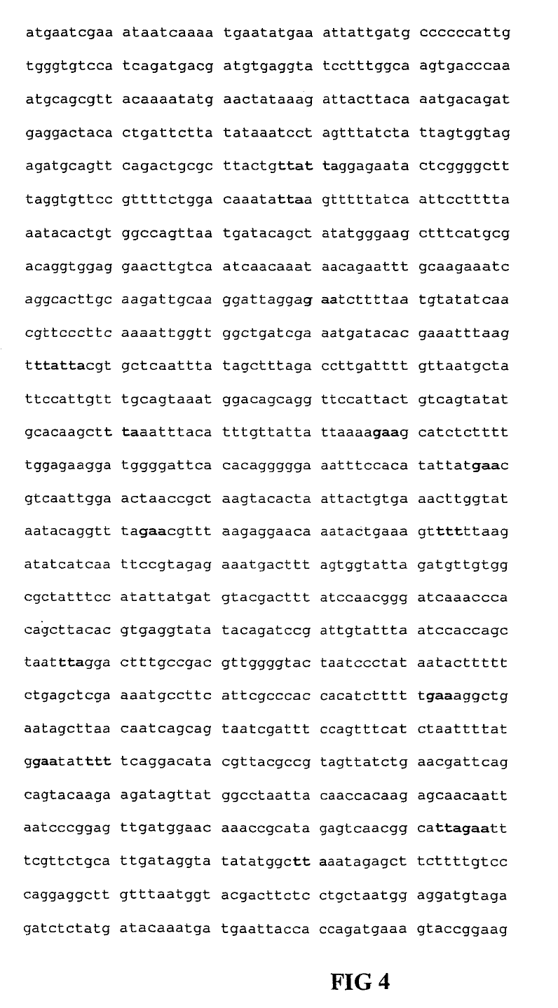

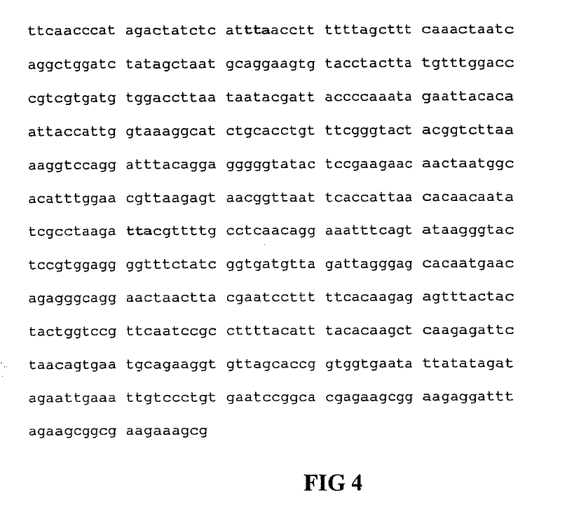

The sequence of a cry9Ca1 gene modified by replacement with respect to the codons encoding the valine, aspartic acid and tryptophan residues up to 25% is given in FIG. 4 (SEQ ID NO:11). The modified bases are in bold.

The sequence of a Cry9Ca1 protein modified by valine, aspartic acid and tryptophan residue replacement up to 25% is given in FIG. 5 (SEQ ID NO:12). The modified amino acids are in bold.

The creation of a modified cry9Ca1 gene in which 25% of the valine, aspartic acid and tryptophan codons have been modified, and the sequence of which is given in FIG. 4 (SEQ ID NO:11), can be carried out using, among those given in FIG. 5 (SEQ ID NOS:94 to 160), the following oligonucleotides:

Oligonucleotide No. 60

Oligonucleotide No. 62

Oligonucleotide No. 67

Oligonucleotide No. 72

Oligonucleotide No. 77

Oligonucleotide No. 78

Oligonucleotide No. 80

Oligonucleotide No. 82

Oligonucleotide No. 83

Oligonucleotide No. 88

Oligonucleotide No. 90

Oligonucleotide No. 92

Oligonucleotide No. 96

Oligonucleotide No. 97

Oligonucleotide No. 103

Oligonucleotide No. 111

The method preferably used is a multiple mutagenesis with a mixture of the oligonucleotides mentioned immediately above. The site-directed mutagenesis procedure is similar to that described in Example 1, the only difference being that a mixture of mutagenesis oligonucleotides is used in this example, whereas a single mutagenesis oligonucleotide is used in Example 1. The protocol used is that described in Examples 1 to 4. It is common to each of the mutagenesis series, only the mutagenesis oligonucleotide and the oligonucleotide for inhibition/restoration of the resistance to the antibiotic change.

### Example 6

### Production of Modified Cry Proteins in B. Thuringiensis and Purification

The native and modified genes are inserted, with their promoter and terminator sequences, into the E. coli-B. thuringiensis pHT3101 shuttle vector (Lereclus et al., 1989).

The plasmid DNA is prepared by minipreparation according to the alkaline lysis technique (Birboim and Doly, 1979). Each bacterial colony is grown in 2 ml of LB medium supplemented with the appropriate antibiotic, overnight at 37° C. with shaking (200 rpm). The culture is then transferred into a microtube and then centrifuged at 13 500 g for 5 min. After removal of the supernatant, the bacteria are resuspended in 100 μl of a solution of 25 mM Tris-HCl, pH 8, 10 mM EDTA containing RNase A at the final concentration of 100 μg/ml. 200 μl of a solution of 0.2 M NaOH, 1% SDS are added and the suspension is mixed twice by inverting the tube. 150 μl of a 2.55 M potassium acetate solution, pH 4.5, are added and the suspension is incubated for 5 min in ice. After centrifugation for 15 min at 13 500 g, the supernatant is transferred into a microtube containing 1 ml of cold ethanol. After centrifugation for 30 min at 13 500 g, the supernatant is removed and the pellet is washed with 1 ml of 70% ethanol. The pellet containing the DNA is dried for a few minutes under vacuum and then taken up in 50 μl of sterile distilled water. The samples are then placed at 65° C. for 30 min.

The digestions with restriction endonucleases are carried out per 1 μg of DNA in a final volume of 20 μl in the presence of one tenth of the final volume of 10× buffer recommended by the supplier for each enzyme and using 5 units of enzyme. The reaction is incubated for 2 to 3 h at the optimum temperature for the enzyme.

Dephosphorylation of the 5′ ends engendered by restriction enzyme is carried out with calf intestine alkaline phosphatase. The reaction is carried out using 5 μl of 10× dephosphorylation buffer (500 mM Tris-HCl, pH 9.3, 10 mM MgCl2, 1 mM ZnCl₂ and 10 mM spermidine) and one unit of enzyme per μg of DNA in a final volume of 50 μl. The reaction is incubated for one hour at 37° C. in the case of overhanging 5′ ends or at 55° C. in the case of blunt ends or 3′ overhanging ends. After dephosphorylation, the enzyme is then inactivated for 30 min at 65° C. and then removed with two volume for volume extractions with a mixture of phenol-chloroform-isoamyl alcohol (25-24-1). The ligations are carried out using T4 phage DNA ligase. They are carried out with an amount of vector equal to 100 ng and an insert/vector molar ratio of between 5 and 10. The final volume of the reaction is 30 μl and comprises 3 μl of 10× ligase buffer (300 mM Tris-HCl, pH 7.8, 100 mM MgCl2, 100 mM DTT and 10 mM ATP) and 3 units of enzyme. The reaction is incubated overnight at 14° C.

The construct is inserted into an acrystalliferous strain of B. thuringiensis according to a method derived from that described in 1989 by Lereclus et al. and described elsewhere (Rang et al., 1999, 2000). A preculture of acrystalliferous Bacillus thuringiensis subsp. kurstaki HD-1 is incubated overnight at 37° C. with shaking in 10 ml of BHI medium (Difco). 250 ml of BHI medium are then inoculated with 5 ml of preculture and incubated at 37° C. with shaking until the OD at 600 nm of the culture reaches the value of 0.3. The culture is then centrifuged at 1 000 g at 4° C. for 10 min. The supernatant is removed and the bacterial pellet is rinsed with 50 ml of cold sterile distilled water. The bacteria are again centrifuged for 10 min at 1 000 g at 4° C. The pellet is taken up in 4 ml of a cold, sterile solution of 40% PEG-6000 and placed in ice. 200 μl of bacteria are then mixed with 5 μg of plasmid DNA and then placed in an electroporation cuvette 0.2 cm in diameter. The cuvette is then placed in the electroporation chamber and a current corresponding to the following characteristics: 2.5 kV, 1 000 Ω, 25 μF, is supplied. The bacteria are then covered, placed in ice for 10 min before being added to 2 ml of BHI medium, and incubated at 37° C. with shaking for 90 min. 200 μl of culture are then plated out onto Petri dishes containing usual solid medium (IEBC, 1994) supplemented with erythromycin at a final concentration of 25 μg/ml, and incubated overnight at 28° C.

The recombinant strains of Bacillus thuringiensis expressing the native gene or the mutated genes are cultured in 250 ml of Usual medium containing 25 μg/ml of erythromycin with shaking at 28° C. The bacterial growth is verified by observation by phase-contrast light microscopy. The bacteria are grown until bacterial lysis after sporulation. The culture is then centrifuged at 5 000 g for 10 min. The pellet is washed with 25 ml of 1 M NaCl and the suspension is again centrifuged at 5 000 g for 10 min. The pellet is then taken up in 15 ml of sterile distilled water containing 1 mM of PMSF, incubated in ice, and treated with ultrasound (100 W) for 1 min in order to dissociate the aggregates between the spores and the crystals. The suspension is then loaded onto a discontinuous NaBr gradient made up of a layer of 4 ml of 38.5% concentration, of 4 layers of 6 ml of 41.9%, 45.3%, 48.9% and 52.7% and a layer of 3 ml of 56.3%. The gradient is then centrifuged at 20 000 g for 90 min at 20° C. The various components of the suspension (spores, cell debris, parasporal bodies) are positioned in the gradient at various levels depending on their density. Each band is recovered and washed three times with one volume of sterile distilled water. Each band is observed by phase-contrast light microscopy. The fraction containing inclusion bodies is stored at −20° C. in sterile distilled water containing 1 mM of PMSF, for subsequent analysis.

### Example 7

### Analysis of the Stability of the Proteins to Proteases

The first stability analysis performed is the verification of stability to trypsin. The proteins present in the parasporal inclusion body are solubilized for one hour at 37° C. in solubilizing buffer (50 mM Na₂CO₃, pH 10.8, 14.6 mM 2-mercaptoethanol). The suspension is then centrifuged at 14 000 g for 10 min in order to remove the insoluble material. One tenth of the total volume of 0.05% trypsin is then added to the supernatant and the mixture is incubated for 2 h at 37° C. The condition of the proteins after trypsin treatment is verified by SDS-polyacrylamide gel analysis according to the Laemmli method (1970). This technique allows the proteins to be separated according to their molecular mass by virtue of the presence of SDS, which confers an overall negative charge on all the proteins. The sample is first treated by adding one volume of 2× treatment solution (125 mM Tris-HCl, 20% glycerol, 4% SDS, 10% 2-mercaptoethanol, 0.01% bromophenol blue) and is then denatured for 5 min in boiling water. The sample is then loaded onto the gel and first passes through a first stacking gel made up of a 4% acrylamide-bisacrylamide mixture, 0.1% SDS, and 125 mM Tris-HCl, pH 6.8. The sample then passes through the separating gel made up of 12% acrylamide-bisacrylamide, 0.1% SDS and 375 mM Tris-HCl, pH 8.8, which makes it possible to separate the various proteins as a function of their size. The electrophoresis is carried out at 100 V in migration buffer (25 mM Tris-HCl, pH 8.3, 192 mM glycine, 0.1% SDS) until the bromophenol blue leaves the gel. The gel is then stained for one hour with a solution of 40% methanol-7% acetic acid containing 0.025% of Coomassie blue and then destained with a 50% methanol-10% acetic acid solution. The gel is ultimately fixed in a 5% methanol-7% acetic acid solution.

The second analysis is the verification of the stability to the digestive juices of insects. The trypsin-stable toxins are purified by FPLC (Pharmacia) using an anion exchange column (Q-Sepharose) equilibrated with a 40 mM Na₂CO₃ solution, pH 10.7. The elution is carried out with a gradient of 50 to 500 mM of NaCl. The OD at 280 nm of the fractions is measured and the fractions containing the proteins are analyzed by SDS-polyacrylamide gel electrophoresis. The fractions containing the toxin are pooled and dialyzed at 4° C. against distilled water for approximately 48 h until the proteins precipitate. The protein suspension is then centrifuged at 8 000 g and at 4° C. for 30 min. The toxins contained in the pellet are resuspended in distilled water and assayed according to Bradford (1976). They are then divided up into aliquot fractions of 100 μg, lyophilized, and then stored at 4° C. Before they are used, the toxins are solubilized and brought to a concentration of 10 mg/ml with 25 mM Tris, pH 9.5, for the purpose of testing their stability to the digestive juices of Ostrinia nubilalis larvae. The digestive juice of the O. nubilalis larvae can be taken either by regurgitation induced by electric shock according to the procedure of Ogiwara et al. (1992), or by dissection of the larvae and collection of the intestinal juice with a pipette according to the method described by Baines et al. (1994). In both cases, between 100 and 200 individuals are required to collect the digestive juice. The juice collected is centrifuged at 15 000 g for 15 minutes at 4° C. before use. The protein concentration of the digestive juice is determined by the Bradford method (BioRad). The reaction is carried out for 15 minutes at 37° C. with a 1:1 ratio (based on the protein concentration of the digestive juice) of toxin to digestive juice. The reaction is stopped with a cocktail of protease inhibitors (Protease Inhibitors Set, Roche Diagnostics) mixed with an equivalent volume of 2× treatment solution (125 mM Tris-HCl, 20% glycerol, 4% SDS, 10% 2-mercaptoethanol, 0.01% bromo-phenol blue), and then incubated for 5 minutes in boiling water. The proteins are then analyzed by SDS-PAGE according to the procedure described above, in order to determine their resistance to the digestive juices of the larvae and their possible state of degradation.

The final type of stability analysis carried out is that of stability to pepsin. The lyophilized native and modified toxins are dissolved in a gastric buffer (0.5 mg NaCl, 1.75 ml 1M HCl in 250 ml H₂O, pH 2.0) simulating mammalian stomach fluid and containing 0.32% of pepsin. Samples are removed after 0, 5, 15, 60 and 240 minutes of incubation at 37° C. and then analyzed by SDS-polyacrylamide gel electrophoresis as described above. These conditions are identical to those described in the EPA (United States Environmental Protection Agency) No. 4458108.

This series of analyses makes it possible to visualize the state of conservation of the native and mutated proteins, and therefore their stability, to various proteases present in insects (trypsin and digestive juices) and, consequently, to verify that the mutated proteins have effectively conserved their stability in insects. These analyses also make it possible to verify that the mutated proteins are effectively degraded by pepsin under the conditions similar to those present in the mammalian stomach.

### Example 8

### Analysis of the Insecticidal Properties

The analysis of insecticidal properties is carried out through two types of experiment for testing the two steps of the process of toxicity in insects: receptor site recognition and evaluation of the toxicity in vivo.

Analysis of the affinity of the toxins for the receptor site is carried out using toxin radiolabeled with iodine 125 (¹²⁵I). The FPLC-purified and lyophilized activated toxins are taken up in storage buffer (20 mM Tris-HCl, pH 8.6) and analyzed by SDS-PAGE in order to verify their condition. An aliquot fraction is assayed according to the Bradford method (1976). The toxins are iodinated according to the chloramine-T method (Markwell, 1982). 25 μμg of toxins are incubated for 5 min at ambient temperature with 0.25 mCi of Na-¹²⁵I and an “Iodo-bead” (Pierce) in 50 μl of sodium carbonate buffer (50 mM Na₂CO₃, pH 10). The iodination reaction is then deposited at the surface of a dextran desalting column (Pierce) equilibrated with CBS buffer (50 mM Na₂CO₃, pH 10.8, 150 mM NaCl) in order to remove the free iodine. The labeling and the quality of the protein are verified by SDS-PAGE followed by autoradiography. The mean specific activity of a labeled toxin is 100 000 cpm/pmol.

In order to prepare the brush border membrane vesicles (BBMV) on which the study of the affinity of the toxins for the receptors is carried out, the insects are allowed to grow to the final larval stage. The insect used is Ostrinia nubilalis, but the methodology used is applicable to any other insect species. The use of another insect species requires the production conditions and the nutritive medium to be adapted to each of the species envisioned, which can be readily done by any individual skilled in the art. The Ostrinia nubilalis larvae are produced on meridic artificial nutritive medium (Lewis and Lynch, 1969; Reed et al., 1972; Ostlie et al., 1984). The method for producing the Ostrinia nubilalis larvae is that described by Huang et al. (1997). The larvae are produced individually in 128-well plates (Bio-Ba-128, C-D International). Each well contains 2 ml of artificial medium. After ten days, the larvae are transferred into larger dishes (18.4 cm in diameter and 7.6 cm high) containing 300 ml of artificial nutritive medium. Corrugated cardboard is placed inside by way of pupation site. During the larval phase, the temperature of the production cell is 25° C. with constant light (24 h). The pieces of cardboard containing the chrysalises are transferred into screened cages for the emergence and the production of the adults. Waxed paper is placed in the case to accept the eggs. The eggs are removed and kept on hold at 15° C. The production of the adults is carried out at 25° C. with 75% relative humidity and a photoperiod of 14 h.

To carry out the tests of affinity of the toxins for the receptor sites, the larvae are collected at the beginning of the 5th larval stage and placed under fasting conditions for 6 hours. They are then removed and placed on ice for 5 minutes. The larvae are dissected and the digestive tube is removed. The dissected digestive tubes are pooled in groups of 20, placed in a cryotube containing MET buffer (300 mM mannitol, 5 mM EGTA, 17 mM Tris-HCl, pH 7.5), frozen in liquid nitrogen and stored at −80° C.

The BBMVs are prepared according to the differential magnesium precipitation method (Wolfersberger et al., 1987; Nielsen-LeRoux and Charles, 1992). The BBMVs are taken up in TBS buffer (20 mM Tris-HCl, pH 8.5, 150 mM NaCl) and the total protein concentration is determined by the Bradford method using the Biorad kit and bovine serum albumin (BSA) as standard (Bradford, 1976).

The in vitro receptor recognition assays are carried out in 1.5 ml polyethylene microtubes, in 20 mM sodium phosphate buffer, pH 7.4, containing 0.15 M of NaCl and 0.1% of bovine serum albumin (PBS/BSA). The assays are carried out, in duplicate, at ambient temperature in a total volume of 100 μl, with 10 μg of BBMV protein. The toxins attached to the BBMVs are separated from the free toxins by centrifugation at 14 000 g for 10 min at ambient temperature. The pellets of each sample, containing the toxin attached to the membrane, are rinsed twice with 200 μl of cold PBS/BSA buffer (20 mM Tris/HCl, 150 mM NaCl, 0.1% BSA, pH 8.5) and then centrifuged. The pellets are finally resuspended in 200 μl of PBS/BSA buffer and added to 3 ml of HiSafe 3 scintillant cocktail (Pharmacia) in a scintillation vial. The counting is performed in a liquid scintillation counter.

The direct binding assays are carried out according to the Nielsen-LeRoux and Charles protocol (1992). 30 μg of BBMV per microtube are incubated with a series of concentrations of 1 to 100 mM of toxin labeled with ¹²⁵I-iodine in Tris/BSA buffer (20 mM Tris/HCl, 150 mM NaCl, 0.1% BSA, pH 8.5). The amount of nonspecific attachment is determined in parallel experiments in the presence of a 300-fold excess of unlabeled toxin. After incubation for 90 minutes at ambient temperature, the samples are centrifuged at 14 000 g for 10 minutes at 4° C. The pellets are rinsed twice with cold Tris/BSA buffer and resuspended in 150 μl of the same buffer and added to 3 ml of HiSafe 3 scintillant cocktail (Pharmacia) in a scintillation vial. Each experiment is carried out in duplicate and each experimental point is counted twice in a liquid scintillation counter. The data are analyzed using the LIGAND software (Munson and Rodbard, 1980) marketed by the company Biosoft.

The homologous competition experiments are carried out as described above for the direct binding experiments, with 10 μg of BBMVs in a total volume of 100 μl for 90 min at ambient temperature. The BBMVs are incubated in a fixed concentration of 10 nM of toxin labeled with 125I-iodine in the presence of a series of concentrations (from 0.1 to 300 times the concentration of the labeled toxin) in Tris/BSA buffer. The value for the nonspecific binding (the binding always present in the presence of a 300-fold excess of the unlabeled toxin) is subtracted from the total value counted. Each experiment is carried out in duplicate and each experimental point is counted twice in a liquid scintillation counter. The data are analyzed using the LIGAND software (Munson and Rodbard, 1980) marketed by the company Biosoft.

The in vivo toxicity assays are carried out according to the procedure described by Lambert et al. (1996). The activated and solubilized toxin is incorporated into the nutritive medium at various concentrations either side of the 50% lethal dose (LD50) of Cry9Ca1 for Ostrinia nubilalis, which is 96.6 ng of toxin per cm² of surface area of medium. Six doses, of 0.1 ng/cm², 1 ng/cm², 10 ng/cm², 100 ng/cm², 1 000 ng/cm² and 10 000 ng/cm² evaluate the LD50 values of the native and modified toxins. The toxicity assays are carried out on neonatal larvae in plates containing 24 wells of 2 cm² (Multiwell-24 plates, Coming Costar Corp.). 50 μl of each of the dilutions of toxin are plated out onto the medium and dried under a flow hood. One larva is placed in each well and a total of 24 larvae is used for each dose (one plate per dose). For each dose the assay is repeated at least three times. A control is carried out with distilled water. The plates are covered and placed at 25° C., 70% relative humidity and with a photoperiod of 16 h. The mortality is controlled after 7 days and the LD50 is calculated according to the probit method (Finney, 1971).

### Bibliographical References

Abdul-Rauf, M. and Ellar, D. J. 1999. Mutations of loop 2 and loop 3 residues in domain II of Bacillus thuringiensis CryC δ-endotoxin affect insecticidal specificity and initial binding to Spodoptera littoralis and Aedes aegypti midgut membranes. Current Microbiology. 39: 94-98.

Aronson, A. I., D. Wu, and C. Zhang. 1995. Mutagenesis of specificity and toxicity regions of a Bacillus thuringiensis protoxin gene. J. Bacteriol. 177: 4059-4065.

Baines, D., A. Brownright and J. L. Schwartz. 1994. Establishment of primary and continuous cultures of epithelial cells from larval lepidopteran midguts. J. Insect. Physiol. 40: 347-357.

Birnboim, H. C. and J. Doly. 1979. A rapid alkaline extraction procedure for screening recombinant plasmid DNA. Nucleic Acids Res. 7: 1513.

Bradford, M. M. 1976. A rapid and sensitive method for the quantitation of micro-gram quantities of protein utilizing the principle of protein-dye binding. Anal. Biochem. 72: 248-254.

Chen, X. J., Lee, M. K. and Dean, D. H. (1993). Site-directed mutations in a highly conserved region of Bacillus thuringiensis δ-endotoxin affect inhibition of short circuit current across Bombyx mori midguts. Proc. Natl. Acad. Sci. USA. 90 9041-9045.

Coux, F., V. Vachon, C. Rang, K. Moozar, L. Masson, M. Royer, M. Bes, S. Rivest, J. L. Schwartz, R. Laprade and R. Frutos. 1999. Role of interdomain salt bridges in the pore-forming ability of insecticidal toxin Cry1Aa of Bacillus thuringiensis. 32nd Annual Meeting of the Society for Invertebrate Pathology. 22-27 Aug. 1999, Irvine, Calif., USA.

Crickmore, N. 2001. epunix.biols.susx.ac.uk/Home/Neil_Crickmore/Bt/list.html

Crickmore, N., Zeigler, D. R., Feitelson, J., Schnepf, E., van Rie, J., Lereclus, D., Baum, J., and Dean, D. H. 1998. Revision of the nomenclature for the Bacillus thuringiensis pesticidal proteins. Microbiol. Mol. Biol. Rev. 62: 807-813.

de Maagd, R. A., P. Bakker, N. Staykov, S. Dukiandjiev, W. Stiekema and D. Bosch. 1999. Identification of Bacillus thuringiensis delta-endotoxin Cry1C domain III amino acid residues involved in insect specificity. Appl. Environ. Microbiol. 65(10): 4369-4374.

Dean, D. H., Rajamohan, F., Lee, M. K., Wu, S. J., Chen, X. J., Alcantara, E., Hussain, S. R. 1996. Probing the mechanism of action of Bacillus thuringiensis insecticidal proteins by site-directed mutagenesis—a minireview. Gene. 179: 111-117.

Finney, D. J. 1971. Probit analysis. A statistical treatment of the sigmoid response curve. University Press, Cambridge.

Gazit, E. and Shai, Y. 1993. Structural and functional characterization of the α5 segment of Bacillus thuringiensis δ-endotoxin. Biochemistry. 32: 3429-3436.

Grochulski, P., Masson, L., Borisova, S., Pusztai-Carey, M., Schwartz, J. -L., Brousseau, R. and Cygler, M. (1995) Bacillus thuringiensis CryIA(a) insecticidal toxin: crystal structure and channel formation. J. Mol. Biol. 254: 447-464.

Guex, N. and Peitsch, M. C. 1997. SWISS-MODEL and the Swiss-Pdb Viewer: An environment for comparative protein modelling. Electrophoresis. 18: 2714-2723.

Huang, F. R., A. Higgins and L. L. Buschman. 1997. Baseline susceptibility and changes in susceptibility to Bacillus thuringiensis subsp. kurstaki under selection pressure in European corn borer (Lepidoptera: Pyralidae). J. Econ. Entomol. 90: 1137-1143.

Hussain, S. R. A., A. I. Aronson and D. H. Dean. 1996. Substitution of residues on the proximal side of Cry1A Bacillus thuringiensis delta-endotoxins affects irreversible binding to Manduca sexta midgut membrane. Biochemical and Biophysical Research Communications. 226(1) 8-14.

Hutchinson, C. A., S. Phillips, M. H. Edgell, S. Gillam, P. Jahnke and M. Smith. 1978. Mutagenesis at a specific position in a DNA sequence. J. Biol. Chem. 253: 6551.

IEBC. 1994. Collection of Bacillus thuringiensis and Bacillus sphaericus (classified by serotypes). Institut Pasteur Paris.

Laemmli, U. K. 1970. Cleavage of structural proteins during the assembly of the head of bacteriophage T4. Nature. 227: 680-685.

Lambert, B., Buysse, L., Decock, C., Jansen, S., Piens, C., Saey, B., Seurinck, J., Van Audenhove K., van Rie, J., van Vliet, A. and Peferoen, M. 1996. A Bacillus thuringiensis insecticidal crystal protein with a high activity against members of the family Noctuidae. Appl. Environ. Microbiol. 62: 80-86.

Lee, M. K., You, T. H., Gould, F. L. and Dean, D. H. 1999. Identification of residues in domain III of Bacillus thuringiensis Cry1Ac toxin that affect binding and toxicity. Appl. Environ. Microbiol. 65: 4513-4520.

Lereclus, D., Arantes, S. O., Chauffaux, J. and Lecadet, M. M. 1989. Transformation and expression of a cloned delta-endotoxin gene in Bacillus thuringiensis. FEMS Microbiol. Lett. 60: 211-217.

Lewis, L. C. and R. E. Lynch. 1969. Rearing the European corn borer on diets containing corn leaf and wheat germ. Iowa Stae J. Sci. 44: 9-14.

Li, J., Carroll, J. and Ellar, D. J. (1991). Crystal structure of insecticidal δ-endotoxin from Bacillus thuringiensis at 2.5 Å resolution. Nature 353: 815-821.

Manoj Kumar, A. S. and Aronson, A. I. 1999. Analysis of mutations in the pore-forming region essential for insecticidal activity of a Bacillus thuringiensis δ-endo-toxin. J. Bacteriol. 181: 6103-6107.

Markwell, M. A. K. 1982. A new solid-state reagent to iodinate proteins. I-Conditions for the efficient labelling of antiserum. Anal. Biochem. 125: 427-432.

Masson, L., B. E. Tabashnik, Y. B. Liu, R. Brousseau and J. L. Schwartz. 1999. Helix 4 of the Bacillus thuringiensis Cry1Aa toxin lines the lumen of the ion channel. J. Biol. Chem. 274: 31996-32000.

Munson, P. J. & Rodbard, D. 1980. LIGAND: A versatile computerized approach for characterization of ligand-binding systems. Anal. Biochem. 107, 220-239.

Nielsen-LeRoux, C., Charles, J. F. 1992. Binding of Bacillus sphaericus binary toxin to a specific receptor on midgut brush border membranes from mosquito larvae. Eur. J Biochem. 210: 585-590.

Ogiwara, K., L. S. Indrasith, S. Asano and H. Hori. 1992. Processing of δ-endotoxin from Bacillus thuringiensis subsp. kurstaki HD-1 and HD-73 by gut-juices of various insect larvae. J. Invert. Pathol. 60: 121-126.

Ostlie, K. R., G. L. Hein, L. G. Higley, L. V. Kaster and Showers, W. B. 1984. European corn borer (Lepidoptera: Pyralidae) development, larval survival, and adult vigor on meridic diets containing marker dies. J. Econ. Entomol. 77: 118-120.

Peitsch, M. C. 1995. Protein modeling by E-mail. Bio/Technology. 13: 658-660.

Peitsch, M. C. 1996. ProMod and Swiss-Model: Internet-based tools for automated comparative protein modelling. Biochem. Soc. Trans. 24: 274-279.

Rajamohan, F., Alcantara, E., Lee, M. K., Chen, X. J., Curtiss, A. and Dean, D. H. (1996). Single amino acid changes in domain II of Bacillus thuringiensis CryIAb δ-endotoxin affect irreversible binding to Manduca sexta midgut membrane vesicles. J. Bacteriol. 177: 2276-2282.

Rajamohan, F., M. K. Lee and D. H. Dean. 1998. Bacillus thuringiensis insecticidal proteins: molecular mode of action. Prog. Nucleic Acid Res. Mol. Biol. 60: 1-27.

Rang, C., L. Lacey and R. Frutos. 2000. The crystal proteins from Bacillus thuringiensis subsp. thompsoni display a synergistic activity against the codling moth, Cydia pomonella. Current Microbiology 40: 200-204.

Rang, C., Vachon, V., Coux, F., Carret, C., Moar, W. J., Brousseau, R., Schwartz, J. L., Laprade, R. and Frutos, R. 2001. Exchange of domain I from Bacillus thuringiensis Cry1toxins influences protoxin stability and crystal formation. Current Microbiology. In Press.

Rang, C., Vachon, V., de Maagd, R. A., Villalon, M., Schwartz, J. -L., Bosch, D., Frutos, R. and Laprade, R. 1999. Interaction between functional domains of Bacillus thuringiensis insecticidal crystal proteins. Appl. Environ. Microbiol. 65: 2918-2925.

Reed, G. L., W. B. Showers, J. L. Huggans and S. W. Carter. 1972. Improved procedures for mass rearing the European corn borer. J. Econ. Entomol. 65: 1472-1476.

Sambrook, J., E. F. Fritch and T. Maniatis. 1989. Molecular Cloning: A Laboratory Manual, Nolan C. ed., New York: Cold Spring Harbor Laboratory Press.

Sanger, F., Nicklen, S. & Coulson, A. R. 1977. DNA sequencing with chain-terminating inhibitors. Proc. Natl. Acad. Sci. USA 74: 5463-5467.

Schnepf, E., N. Crickmore, J. van Rie, D. Lereclus, J. Baum, J. Feitelson, D. R. Zeigler and D. H. Dean. 1998. Bacillus thuringiensis and its pesticidal crystal proteins. Microbiol. Mol. Biol. Rev. 62: 775-806.

Terra, W. B. and C. Ferreira. 1994. Insect digestive enzymes: properties, compartmentalization and function. Comp. Biochem. Physiol. 109B: 1-62.

Vachon, V. F., Coux, F., G. Prefontaine, C. Rang, L. Marceau, L. Masson, R. Brousseau, R. Frutos, J. L. Schwartz and R. Laprade, R. 2000. Role of α-helix three charged residues in pore formation by the Bacillus thuringiensis insecticidal toxin Cry1Aa. 33rd Annual Meeting of the Society for Invertebrate Pathology. 13-18 Aug. 2000, Guanajuato, Mexico.

Wolfersberger, M. G., Luthy, P., Maurer, A., Parenti, P., Sacchi, V., Giordana B., Hanozet, G. 1987. Preparation and partial characterisation of amino acid transporting brush border membrane vesicles from the larval midgut of the cabbage butterfly (Pieris brassicae). Comp. Biochem. Physiol. 86: 301-308.

Wu, S. J. and Dean D. H. 1996. Functional significance of loops in the receptor binding domain of Bacillus thuringiensis CryIIIA δ-endotoxin. J. Mol. Biol. 255: 628-640.

1. A pepsin-sensitive modified Cry protein, characterized in that it has at least one additional pepsin cleavage site.

2. The modified Cry protein as claimed in claim 1, characterized in that the additional pepsin cleavage site is represented by an amino acid residue chosen from leucine, phenylalanine and glutamic acid residues.

3. The modified Cry protein as claimed in either of claims 1 and 2, characterized in that it is selected from the Cry1, Cry3, Cry4, Cry7, Cry8, Cry9, Cry10, Cry16, Cry17, Cry19 and Cry20 proteins.

4. The modified Cry protein as claimed in claim 3, characterized in that it is a Cry9C protein.

5. The modified Cry protein as claimed in claim 4, characterized in that it is the Cry9Ca1 protein.

6. The modified Cry protein as claimed in one of claims 1 to 5, characterized in that it has at least one additional pepsin cleavage site in at least one of the inter-α-helix loops of domain I.

7. The modified Cry protein as claimed in one of claims 1 to 6, characterized in that it has at least one additional pepsin cleavage site in the inter-α-helix loop linking the α3 and α4 helices of domain I.

8. The modified Cry protein as claimed in one of claims 5 to 7, characterized in that it has an additional pepsin cleavage site at position 164.

9. The modified Cry protein as claimed in claim 8, characterized in that it is selected from the Cry proteins, the sequences of which are represented by the identifiers SEQ ID NO:4, SEQ ID NO:6 or SEQ ID NO:8.

10. The modified Cry protein as claimed in one of claims 1 to 5, characterized in that the additional pepsin cleavage sites are introduced by substituting aspartic acid residues with glutamic acid residues, substituting tryptophan residues with phenylalanine residues, and substituting valine or isoleucine residues with leucine residues.

11. The modified Cry protein as claimed in claim 11, characterized in that the degree of substitutions which said Cry protein possesses is 25%.

12. A method for increasing the pepsin sensitivity of the Cry proteins, characterized in that at least one additional pepsin or cleavage site is introduced into said Cry proteins.

13. The method as claimed in claim 12, characterized in that the additional pepsin cleavage site introduced is represented by an amino acid chosen from leucine, phenylalanine and glutamic acid residues.

14. The method as claimed in either of claims 12 and 13, characterized in that it applies to the Cry proteins selected from the Cry1, Cry3, Cry4, Cry7, Cry8, Cry9, Cry10, Cry16, Cry17, Cry19 and Cry20 proteins.

15. The method as claimed in claim 14, characterized in that it applies to the Cry9C protein.

16. The method as claimed in claim 15, characterized in that it applies to the Cry9Ca1 protein.

17. The method as claimed in one of claims 12 to 16, characterized in that at least one additional pepsin cleavage site is introduced into at least one of the inter-α-helix loops of domain I of said Cry proteins.

18. The method as claimed in one of claims 12 to 17, characterized in that at least one additional pepsin cleavage site is introduced into the inter-α-helix loop linking the α and α4 helices of domain I.

19. The method as claimed in one of claims 16 to 18, characterized in that an additional pepsin cleavage site is introduced at position 164.

20. The method as claimed in one of claims 12 to 16, characterized in that the additional pepsin cleavage sites are introduced by substituting aspartic acid residues with glutamic acid, substituting tryptophan residues with phenylalanine residues, and substituting valine or isoleucine residues with leucine residues.

21. The method as claimed in claim 20, characterized in that the degree of substitution which said Cry protein possesses is less than or equal to 25%.

22. A polynucleotide encoding a modified Cry protein as claimed in one of claims 1 to 11.

23. A chimeric gene comprising, functionally linked to one another, at least:
(a) one promoter which is functional in a host organism
(b) a polynucleotide as claimed in claim 22
(c) a terminator element which is functional in a host organism.

24. The chimeric gene as claimed in claim 23, characterized in that the promoter and the terminator element are functional inplants.

25. An expression or transformation vector containing a chimeric gene as claimed in either of claims 23 and 24.

26. The vector as claimed in claim 27, characterized in that it is a plasmid, a phase or a virus.

27. A host organism transformed with one of the vectors as claimed in either of claims 25 and 26.

28. The host organism as claimed in claim 27, characterized in that it is a plant.

29. The plant as claimed in claim 28, characterized in that it contains, in addition to a chimeric gene as claimed in either of claims 23 and 24, at least one other chimeric gene containing a polynucleotide encoding a protein of interest.

30. A part of a plant as claimed in claim 29.

31. A seed from a plant as claimed in claim 29.

32. A method for producing the modified Cry proteins as claimed in one of claims 1 to 11, characterized in that it comprises at least the steps of:
(a) culturing a transformed host organism according to the invention in a culture medium suitable for the growth and for the multiplication of said organism,
(b) (b) extracting the Cry proteins produced by the transformed organism cultured in step (a).

33. The method as claimed in claim 32, characterized in that it comprises a step (c) of purification of the Cry proteins extracted in step (b).

34. The method as claimed in either of claims 32 and 33, characterized in that the host organism is a microorganism.

35. The method as claimed in claim 34, characterized in that the host organism is a Bacillus thuringiensis bacterium.

36. A monoclonal or polyclonal antibody, characterized in that it is directed against a modified Cry protein as claimed in one of claims 1 to 11.
